(19) United States
(12) Patent Application Publication
(10) Pub. No.: US 2015/0004110 A1
(43) Pub. Date: Jan. 1, 2015

(54) NOVEL ANTI-AGING AND DEPIGMENTING COSMETIC COMPOSITIONS

(71) Applicants: Natacha Frison, Verneuil En Halatte (FR); Benoit Folleas, Senlis (FR); Jean-Louis Brayer, Nanteuil Le Haudouin (FR); Florence Wafflart, Dury (FR)

(72) Inventors: Natacha Frison, Verneuil En Halatte (FR); Benoit Folleas, Senlis (FR); Jean-Louis Brayer, Nanteuil Le Haudouin (FR); Florence Wafflart, Dury (FR)

(73) Assignee: DIVERCHIM, MONTATAIRE (FR)

(21) Appl. No.: 14/367,712

(22) PCT Filed: Dec. 18, 2012
(86) PCT No.: PCT/FR2012/052964

§ 371 Date: Jul. 3, 2014

(30) Foreign Application Priority Data
Dec. 22, 2011 (FR) 11/62289

(51) Int. Cl. A61K 8/60; A61K 8/42; A61K 8/41; A61Q 19/08; A61K 8/40; A61K 8/33; A61K 8/34; A61Q 19/02; A61K 8/35

(52) U.S. Cl. CPC A61K 8/60; A61Q 19/02; A61K 8/42; A61K 8/411; A61K 8/35; A61K 8/40; A61K 8/33; A61K 8/347; A61Q 19/08
USPC 424/62; 514/35; 514/563; 514/646; 514/685; 514/716; 514/717; 514/721; 536/4.1; 564/219; 564/443; 568/331; 568/585; 568/631; 568/647

(57) ABSTRACT

The 1-aryl-2-aryloxyethane compounds of Formula 1

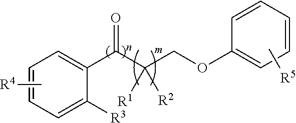

in which n and m are zero or equal to 1, provided that n+m=1, R¹ and R² in particular representing hydrogen atoms, R³ and R⁴ in particular representing hydrogen atoms, halogen atoms, hydroxy or alkoxy groups, and R⁵ in particular representing hydrogen atoms, hydroxy, alkyl, alkoxy, amino or nitro groups, in cosmetic compositions intended for anti-ageing and/or depigmenting and/or anti-inflammatory and/or wound-healing care.

The invention relates to novel anti-ageing and depigmenting cosmetic compositions. They contain 1-aryl-2-aryloxyethanes, representing a family of compounds with useful biological properties.

In 1989, Moinet et al. described the synthesis of a family of novel 1-aryl-2-aryloxyethanes (Lipha, FR2653119) with diuretic, antihypertensive, anti-platelet and anti-lipoxygenase properties. In particular, 2-[2-(4-fluoro-phenoxy)-ethyl]-phenol is a diuretic and antihypertensive agent in its sulphated form (R. P. Garay, J. P. Labaune, D. Mésangeau, C. Nazaret, T. Imbert, G. Moinet, J. Pharm. Exp. Therapeutics, 1990, 255, 415-422).

Other compounds such as derivatives of pyrocatechin-carboxylic acid (Hoffmann Laroche, EP0310126) for treating cardiovascular and bronchopulmonary diseases, and such as derivatives containing a heterocycle (Pfizer, U.S. Pat. No. 5,248,685), for treating asthma, were also described in the 1990s.

In 2006, Bayer described the use of certain derivatives for their anti-parasitic properties (WO119876).

Certain 1-aryl-2-aryloxyethanes are reaction intermediates in the preparation of phenoxypropanolamines, compounds that are antagonists of the β₁ and β₂ forms of adrenaline (S. N. Louis, T. L. Nero, D. Iakovidis, F. M. Colagrande, G. P. Jackman, W. J. Louis, Eur. J. Med. Chem., 1999, 34, 919-937).

It therefore appears that these compounds and their numerous sub-families on the one hand have useful biological properties, and on the other hand have not been investigated so far in the topical field.

One purpose of the invention is to use 1-aryl-2-aryloxyethanes or derivatives thereof in cosmetic compositions.

Another purpose of the invention is to use 1-aryl-2-aryloxyethanes or derivatives thereof in cosmetic compositions intended for anti-ageing and/or depigmenting care.

Another purpose of the invention is to provide a process for preparing novel 1-aryl-2-aryloxyethanes.

According to a general aspect, the invention relates to the use of the compounds of Formula I

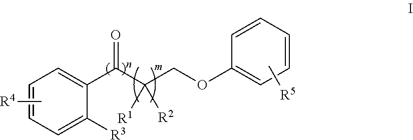

in which

n is equal to 0 or 1, m is equal to 0 or 1, provided that n+m=1,

R¹ and R², which may be identical or different, represent
hydrogen atoms,
halogen atoms selected from fluorine, chlorine, bromine or iodine,
linear or branched alkyl groups comprising from 1 to 10 carbon atoms, in particular the methyl, ethyl, isopropyl groups, optionally substituted with one or more halogens selected from fluorine, chlorine, bromine or iodine, with a —CF₃ group, with a hydroxy,
—OH groups,
linear or branched alkoxy groups comprising from 1 to 10 carbon atoms, in particular methoxy, ethoxy, optionally substituted with one or more halogens selected from fluorine, chlorine, bromine or iodine, and in particular the trifluoromethoxy group —OCF₃,
phenoxy groups —OPh,
aryloxy groups —OAr in which Ar represents an aromatic group comprising from 6 to 12 carbon atoms optionally substituted with one or more halogen atoms selected from fluorine, chlorine, bromine, or optionally substituted with one or more —OH groups in free or protected form, and in particular protected in the form of —OMes group, —OTHP group of formula

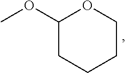

group derived from ethylene glycol of formula

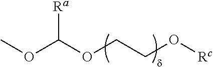

in which
δ varies from 1 to 12,
R^{a} represents a hydrogen, a linear or branched alkyl group comprising from 1 to 6 carbon atoms,
R^{c} represents a linear or branched alkyl group with 1 to 10 carbon atoms,
group derived from propylene glycol of formula,

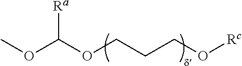

in which

δ′ varies from 1 to 5,
R^{a} and R^{c} have the meanings stated above,
group derived from a glycoside compound that may be α- or β-furanose or α- or β-pyranose, siloxy group of formula —OSi(R^{a})₃ in which R^{a} has the meanings stated above,
—OSitBdPh group of formula

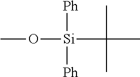 [SMILES: CO[Si](c1ccccc1)(c1ccccc1)C(C)(C)C]

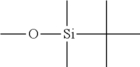

or —OSitBdM group of formula,
benzyloxy groups —OCH₂Ph,
alkenyl groups comprising from 2 to 10 carbon atoms, linear or branched and comprising at least one C═C double bond,
acyloxy groups comprising from 2 to 10 carbon atoms, derived from carboxylic acids,
—OTHP groups of the above formula,
—OMes groups,
groups derived from ethylene glycol of formula

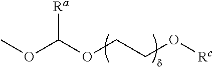

in which
δ varies from 1 to 12,
R^{a} represents a hydrogen or a linear or branched alkyl group comprising from 1 to 6 carbon atoms,
R^{c} represents a linear or branched alkyl group with 1 to 10 carbon atoms,
groups derived from propylene glycol of formula,

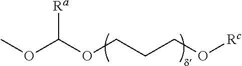

in which
δ′ varies from 1 to 5,
R^{a} and R^{c} have the meanings stated above,
—OH groups optionally coupled to glycoside compounds that may be α- or β-furanose or α- or β-pyranose,
siloxy groups of formula —OSi(R^{a})₃ in which R^{a} has the meanings stated above,
—OSitBdPh groups of formula

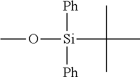 [SMILES: CO[Si](c1ccccc1)(c1ccccc1)C(C)(C)C]

—OSitBdM groups of formula,

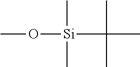

said R¹ and R² optionally forming a ring comprising 3 carbon atoms, said ring having the following formula

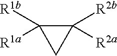

in which
R^{1a}, R^{1b}, R^{2a} and R^{2b}, which may be identical or different, represent
hydrogen atoms,
halogen atoms selected from fluorine, chlorine, bromine or iodine,
linear or branched alkyl groups comprising from 1 to 10 carbon atoms, in particular the methyl, ethyl, isopropyl groups, optionally substituted with one or more halogens selected from fluorine, chlorine, bromine or iodine, with a —CF₃ group, with a hydroxy, provided that R³ is a hydroxy group,

R³ and R⁴, which may be identical or different, represent
hydrogen atoms,
halogen atoms selected from fluorine, chlorine, bromine,
linear, branched or cyclic alkyl groups comprising from 1 to 10 carbon atoms, in particular the methyl, ethyl, isopropyl groups, optionally substituted with one or more halogens selected from fluorine, chlorine, bromine or iodine, with a —CF₃ group, with a hydroxy,
hydroxyl groups —OH,
linear or branched alkoxy groups comprising from 1 to 10 carbon atoms, in particular the methoxy, ethoxy, isopropoxy, tert-butoxy groups, optionally substituted with one or more halogens selected from fluorine, chlorine, bromine or iodine, and in particular the trifluoromethoxy group —OCF₃,
benzyloxy groups,
an alkenyloxy group comprising from 2 to 10 carbon atoms, linear or branched and comprising at least one C═C double bond,
an alkynyloxy group comprising from 2 to 10 carbon atoms, linear or branched and comprising at least one C≡C triple bond,

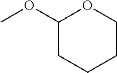

—OTHP groups of formula
substituted —OTHP groups of the following formula

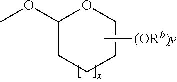

in which
x is equal to 0 or 1, y varies from 0 to 4,
R^{b} is a hydrogen or an acetyl —C(O)—CH₃,
groups derived from ethylene glycol of formula

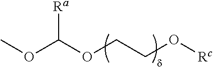

in which
δ varies from 1 to 12,
R^{a} represents a linear or branched alkyl group comprising from 1 to 6 carbon atoms,
R^{c} represents a linear or branched alkyl group with 1 to 10 carbon atoms,
groups derived from propylene glycol of formula

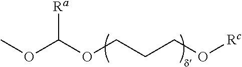

in which
δ′ varies from 1 to 5,
R^{a} and R^{c} have the meanings stated above,
—OH groups optionally coupled to a glycoside compound, which may be an α- or β-furanose or an α- or β-pyranose,
a siloxy group of formula —OSi(R^{a})₃ in which R^{a} has the meanings stated above,
—OSitBdPh of formula

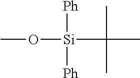 [SMILES: CO[Si](c1ccccc1)(c1ccccc1)C(C)(C)C]

—OSitBdM of formula,

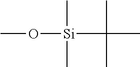

R⁵ represents
a hydrogen atom,
a halogen atom selected from fluorine, chlorine, bromine,
a linear, branched or cyclic alkyl group comprising from 1 to 10 carbon atoms, in particular a methyl, ethyl, isopropyl group, optionally substituted with one or more halogens selected from fluorine, chlorine, bromine or iodine, with a —CF₃ group, with a hydroxy,
an alkenyl group comprising from 2 to 6 carbon atoms, linear or branched and comprising at least one C═C double bond,
an alkynyl group comprising from 2 to 6 carbon atoms, linear or branched and comprising at least one C≡C triple bond,
a phenyl group optionally substituted with a halogen atom, a —CF₃ group, a linear or branched alkyl group comprising from 1 to 10 carbon atoms, a linear or branched alkoxy group comprising from 1 to 10 carbon atoms,
a hydroxyl group —OH,
a linear or branched alkoxy group comprising from 1 to 10 carbon atoms, in particular the methoxy, ethoxy, isopropoxy, tert-butoxy groups, optionally substituted with one or more halogens selected from fluorine, chlorine, bromine or iodine, and in particular the trifluoromethoxy group —OCF₃,
a benzyloxy group,
a phenoxy group,
a thiophenyl group,
an acyloxy group comprising from 2 to 10 carbon atoms, linear or branched,
an —OTHP group of formula

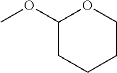

a substituted —OTHP group of the following formula

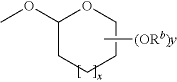

in which
x is equal to 0 or 1, y varies from 0 to 4,
R^{b} is a hydrogen or an acetyl —C(O)—CH₃,
an —OSO₂R^{c} group in which R^{c} is an alkyl group comprising from
   1 to 6 carbon atoms, in particular methyl or ethyl, or an aromatic group comprising from 6 to 18 carbon atoms, optionally substituted with one or more halogen atoms, hydroxy or alkoxy group, nitro,
an —OSO₃M group in which M represents an Na⁺ or K⁺ ion,
a group derived from ethylene glycol of formula

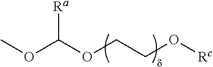

in which
δ varies from 1 to 12,
R^{a} represents a linear or branched alkyl group comprising from 1 to 6 carbon atoms,
R^{c} represents a linear or branched alkyl group with 1 to 10 carbon atoms,
a group derived from propylene glycol of formula

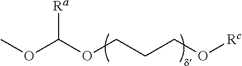

in which
δ′ varies from 1 to 5,
R^{a} and R^{c} have the meanings stated above,
an —OH group optionally coupled to a glycoside compound, which may be an α- or β-furanose or an α- or β-pyranose,
a siloxy group of formula —OSi(R^{a})₃ in which R^{a} has the meanings stated above,
—OSitBdPh of formula

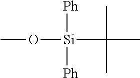 [SMILES: CO[Si](c1ccccc1)(c1ccccc1)C(C)(C)C]

—OSitBdM of formula,

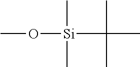

—COOH,
—CN,
—NH₂,
—NH₃⁺, X⁻
—NR^{d}R^{e},
—NHR^{d}R^{e}+, X⁻
—NHCOR^{f},
—NHCOOR^{g},
—NO₂,

R^{d}, R^{e} representing linear or branched alkyl groups comprising from 1 to 4 carbon atoms, optionally substituted with one or more halogen atoms, or carbon chains interrupted by oxygen or sulphur atoms, benzyl groups optionally substituted with a halogen atom, a hydroxy group, an alkoxy group comprising from 1 to 8 carbon atoms,

R^{f} represents a linear or branched alkyl group containing from 1 to 4 carbon atoms, a phthalimido group (in this case NH is replaced with N), a benzyl group optionally substituted with a halogen atom, a hydroxy group, an alkoxy group and in particular substituted with the methoxy group in the para position,

R^{g} represents a linear or branched alkyl group comprising from 1 to 4 carbon atoms, optionally substituted with one or more halogen atoms, or a carbon chain interrupted by oxygen or sulphur atoms, a phenyl group, a benzyl group optionally substituted with a halogen atom, a hydroxy group, an alkoxy group and in particular substituted with the methoxy group in the para position,

X⁻ representing a halide ion,
a group derived from piperazine, in particular

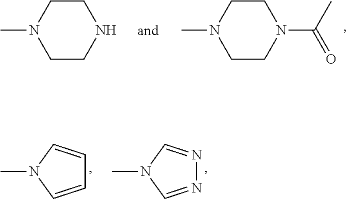

provided that R³ and R⁴ do not represent simultaneously —OH groups or groups comprising an oxygen atom bound to the phenyl,
said compounds of Formula I being used in racemic form or as a single enantiomer for preparing cosmetic compositions having anti-ageing and/or depigmenting and/or wound-healing and/or anti-inflammatory properties.

According to a particular aspect, said compounds of Formula I are used in racemic form or as a single enantiomer for preparing cosmetic compositions intended for anti-ageing and/or depigmenting care.

The compounds of Formula I belong to a family of compounds called “1-aryl-2-aryloxyethanes”.

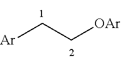

“Aryl” denotes an aromatic group comprising 6 carbon atoms, optionally substituted with the R³ and R⁴ groups, and attached to the ethane chain by carbon 1.
These groups may both be equal to the hydrogen atom; in this case the aryl group is a phenyl group, —C₆H₅.
If the R³ and R⁴ groups are not both equal to the hydrogen atom, then the aryl group in position 1 of the formula shown above is monosubstituted.
If the R³ and R⁴ groups are both different from the hydrogen atom, then the aryl group in position 1 of the formula shown above is disubstituted.

R³ and R⁴ have the meanings designated above and in particular represent H, —OH, —OCH₃, —F, —Br, —OC(O)CH₃.

With the proviso “ . . . R³ and R⁴ do not represent simultaneously —OH groups or groups comprising an oxygen atom bound to the phenyl”, the aryl group derived from benzene-1,2-diol is excluded, in particular pyrocatechol is excluded, as well as its derivatives in which the —OH group would be in another form, in particular a protected form such as the ester form. “Aryloxy” denotes an aryl group attached to carbon 2 of the ethane chain by an oxygen atom. It is optionally monosubstituted with an R⁵ group represented in general formula I and having the meanings designated above.
R⁵ may be equal to the hydrogen atom. In this case, the aryloxy group is a phenoxy group, —OC₆H₅.
R⁵ may be different from the hydrogen atom. In this case, R⁵ is in particular equal to —OH, —F, a nitrogen-containing group, —CH₃, —O—CH₃, —O—CH₂-Ph.

R¹ and R² have the meanings designated above and in particular represent hydrogen atoms or —OH groups.

In the general formula, “n” and “m” are equal to 0 or 1. Their sum is always equal to 1.
If n=0 then m=1: the compound is a 1-aryl-2-aryloxyethane.
If n=1 then m=0: the compound is a ketone.
   In the expression “1-aryl-2-aryloxyethanes or derivatives thereof”, the term “derivatives” corresponds to the case where n=1 and m=0: the compound is a ketone.
   “Cosmetic compositions” denotes mixtures containing at least one active compound intended for care of the body and in particular of the skin.
   “Anti-ageing properties” denotes the set of properties of the cosmetic compositions by which ageing of the skin may be combated, in particular by dermal reinforcement and by improving epidermal differentiation.
   “Anti-inflammatory properties” denotes the set of properties of the cosmetic compositions by which it is possible to combat the secretion of molecules associated with inflammation of the skin, such as the secretion of prostaglandin PGE₂.
   “Depigmenting properties” denotes the set of properties of the cosmetic compositions by which melanogenesis or the biosynthesis of melanocytes may be decreased or inhibited.
   “Wound-healing properties” denotes the set of properties of the cosmetic compositions by which the proliferation and migration of fibroblasts and/or keratinocytes to a wound may be increased.

A particular aspect of the invention relates to the use of the compounds of Formula II

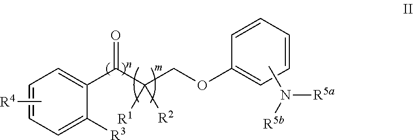

in which

n is equal to 0 or 1, m is equal to 0 or 1, provided that n+m=1,

R¹, R², R³ and R⁴ have the meanings designated above,

—NR^{5a}R^{5b} represents a group comprising a nitrogen atom fixed to the ring, optionally in the form of its salt, this group being selected from
—NH₂,
—NR^{d}R^{e},
—NHCOR^{f},
—NHCOOR^{g},
—NO₂,
a group derived from piperazine, in particular

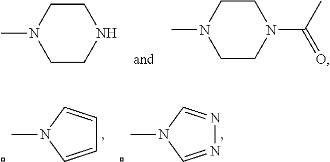

R^{d}, R^{e} representing linear or branched alkyl groups comprising from 1 to 4 carbon atoms, optionally substituted with one or more halogen atoms, or carbon chains interrupted by oxygen or sulphur atoms, benzyl groups optionally substituted with a halogen atom, a hydroxy group, an alkoxy group comprising from 1 to 8 carbon atoms,

R^{f} represents a linear or branched alkyl group containing from 1 to 4 carbon atoms, a phthalimido group (in this case NH is replaced with N), a benzyl group optionally substituted with a halogen atom, a hydroxy group, an alkoxy group and in particular substituted with the methoxy group in the para position,

R^{g} represents a linear or branched alkyl group comprising from 1 to 4 carbon atoms, optionally substituted with one or more halogen atoms, or a carbon chain interrupted by oxygen or sulphur atoms, a phenyl group, a benzyl group optionally substituted with a halogen atom, a hydroxy group, an alkoxy group and in particular substituted with the methoxy group in the para position.

In particular, compounds belonging to the family of the 1-aryl-2-aryloxyethanes and comprising a nitrogen-containing group as substituent of the aryloxy group, have been prepared.
“Group comprising a nitrogen atom fixed to the ring” denotes a nitro function, primary, secondary or tertiary amine or salts thereof, an amide or carbamate function.
This nitrogen-containing group may occupy the ortho, meta or para positions.

Another particular aspect of the invention relates to the use of the compounds of Formula III

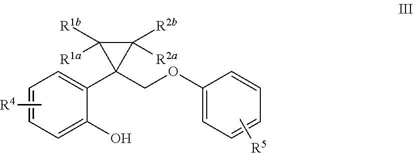

in which

R⁴ and R⁵ have the meanings designated above,

R^{1a}, R^{1b}, R^{2a} and R^{2b}, which may be identical or different, represent
hydrogen atoms,
halogen atoms selected from fluorine, chlorine, bromine or iodine,
linear or branched alkyl groups comprising from 1 to 10 carbon atoms, in particular the methyl, ethyl, isopropyl groups, optionally substituted with one or more halogens selected from fluorine, chlorine, bromine or iodine, with a —CF₃ group, with a hydroxy.
   In the formula shown below, carbon 1 of the ethane chain bears a cyclopropyl group. This cyclopropyl group may itself be substituted with the R^{1a}, R^{1b}, R^{2a} and R^{2b} groups, which may be identical or different, the meanings of which are given above. R^{1a}, R^{1b}, R^{2a} and R^{2b} are in particular four hydrogen atoms.

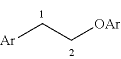

These cyclopropyls have an —OH on the aromatic group in the α position of the carbon bearing the cyclopropyl group.
This cyclopropyl group may itself be substituted with the R^{1a}, R^{1b}, R^{2a} and R^{2b} groups, which may be identical or different, the meanings of which are given above. R^{1a}, R^{1b}, R^{2a} and R^{2b} are in particular four hydrogen atoms.

Another particular aspect of the invention relates to the use of the compounds of Formula 1V

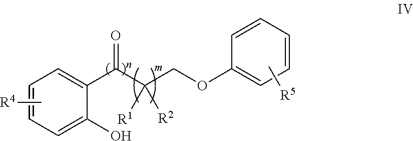

in which

n is equal to 0 or 1, m is equal to 0 or 1, provided that n+m=1,

R¹, R², R⁴ and R⁵ have the meanings designated above.

Compounds IV are characterized by a hydroxy group specifically in the ortho position on the phenyl.

According to an advantageous embodiment of the invention, the compounds of Formula 1VA are used

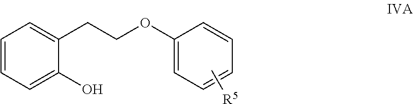

in which R⁵ has the meaning designated above.
Compounds IVA are characterized by a hydroxy group specifically in the ortho position on the phenyl in which the R¹, R² and R⁴ groups are three hydrogen atoms.
R⁵ may occupy the ortho, meta or para positions.

Another particular aspect of the invention relates to the use of the compounds of Formula 1

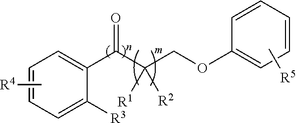

in which

n is equal to 0 or 1, m is equal to 0 or 1, provided that n+m=1,

R¹, R², R³, R⁴ and R⁵ have the meanings designated above,

provided that R³ and R⁴ are both different from —OH or are of any form in which —OH is protected.
The compounds derived from catechol, already described, are excluded.

Another particular aspect of the invention relates to the use of the compounds of Formula V

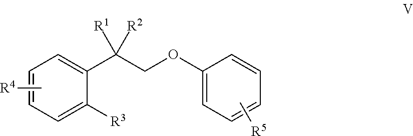

in which

R¹, R², R³, R⁴ and R⁵ have the meanings designated above,

provided that R³ and R⁴ are both different from —OH or are of any form in which —OH is protected.
The ketones bearing a group derived from catechol are excluded.
R⁴ and R⁵ may occupy the ortho, meta or para positions.

Another particular aspect of the invention relates to the use of the compounds of Formula VI

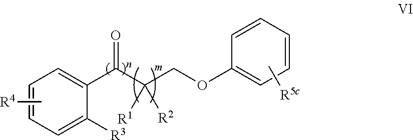

in which

n is equal to 0 or 1, m is equal to 0 or 1, provided that n+m=1,

R¹, R², R³ and R⁴ have the meanings designated above,

R^{5c} is a halogen atom selected from fluorine, chlorine or bromine

The compounds of Formula VI have a halogen fixed on the —OAr group described in the following general formula:
R⁴ and R^{5c} may occupy the ortho,

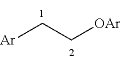

meta or para positions.

According to an advantageous embodiment of the invention, compounds of Formula VII are used

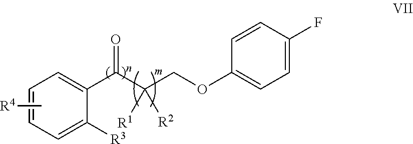

in which

n is equal to 0 or 1, m is equal to 0 or 1, provided that n+m=1,

R¹, R², R³ and R⁴ have the meanings designated above.

Among the compounds having a halogen fixed on the —OAr group of the formula

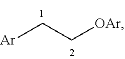

the compounds of Formula VII are characterized by a fluorine atom in the para position.
R⁴ may occupy the ortho, meta or para positions.

Another particular aspect of the invention relates to the use of the compounds of Formula VIII

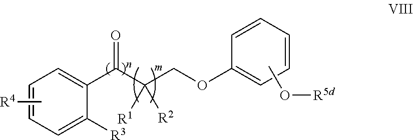

in which

n is equal to 0 or 1, m is equal to 0 or 1, provided that n+m=1,

R¹, R², R³ and R⁴ have the meanings designated above,

—O—R^{5d} is a group selected from
—OH,
linear or branched alkoxy groups comprising from 1 to 10 carbon atoms, in particular the methoxy, ethoxy, isopropoxy, tert-butoxy groups, optionally substituted with one or more halogens selected from fluorine, chlorine, bromine or iodine, and in particular the trifluoromethoxy group —OCF₃,
benzyloxy groups,
phenoxy groups,
—OTHP groups of formula

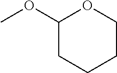

substituted —OTHP groups of the following formula

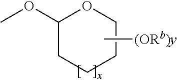

in which
x is equal to 0 or 1, y varies from 0 to 4,
R^{b} is a hydrogen or an acetyl —C(O)—CH₃,
—OSO₂R^{c} groups in which R^{c} is an alkyl group comprising from 1 to 6 carbon atoms, in particular methyl or ethyl, or an aromatic group comprising from 6 to 18 carbon atoms, optionally substituted with one or more halogen atoms, hydroxy or alkoxy group, nitro,
—OSO₃M groups in which M represents an Na⁺ or K⁺ ion,
groups derived from ethylene glycol of formula

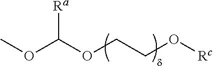

in which
δ varies from 1 to 12,
R^{a} represents a linear or branched alkyl group comprising from 1 to 6 carbon atoms,
R^{c} represents a linear or branched alkyl group with 1 to 10 carbon atoms,
groups derived from propylene glycol of formula

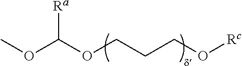

in which
δ′ varies from 1 to 5,
R^{a} and R^{c} have the meanings stated above,
—OH groups optionally coupled to a glycoside compound, which may be an α- or β-furanose or an α- or β-pyranose.
   The compounds of Formula VIII have an oxygen fixed on the —OAr group described in the following general formula:

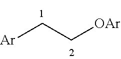

This oxygen-containing group has the meanings stated above. It in particular represents a hydroxy group, —OCH₃, —O—CH₂Ph.

Another particular aspect of the invention relates to the use of the compounds of Formula 1X

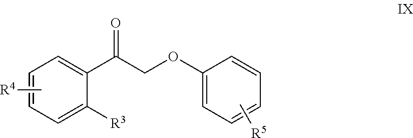

in which R³, R⁴ and R⁵ have the meanings designated above.
According to general formula I, n=1 and m=0, carbon 1 of the formula

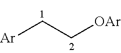

is in this case a carbonyl group: the compounds of Formula III are 1-aryl-2-aryloxyketones.

According to an advantageous embodiment of the invention, compounds of Formula 1XA are used

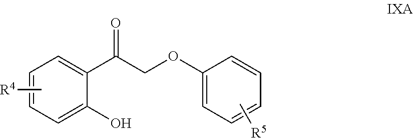

in which R⁴ and R⁵ have the meanings designated above.
These ketones comprise an —OH in the ortho position on the aromatic group in α of the carbonyl.
R⁴ may occupy the meta, para positions or the other ortho position of the ring; R⁵ may occupy the ortho, meta or para positions of the ring.

According to a particular aspect of the invention, compounds of Formula X are used

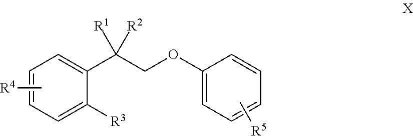

in which R¹, R², R³, R⁴ and R⁵ have the meanings designated above.
The carbon 1 of the formula

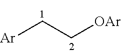

is in this case a methylene group: the compounds of Formula X are 1-aryl-2-aryloxyethanes, optionally substituted with the R¹ and R² groups, the meanings of which are designated above, R¹ and R² in particular being equal to hydrogen atoms, the ketones thus being excluded here.
R⁴ and R⁵ may occupy the ortho, meta or para positions.

According to an advantageous embodiment of the invention, compounds of Formula XA are used

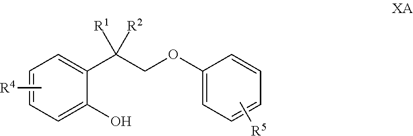

in which R¹, R², R⁴ and R⁵ have the meanings designated above.
These compounds, not belonging to the family of the ketones, comprise an —OH in the ortho position on the —Ar group situated in a of carbon 1 of the formula

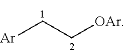

R⁴ may occupy the meta, para positions or the other ortho position of the ring; R⁵ may occupy the ortho, meta or para positions of the ring.

According to another particular aspect of the invention, compounds of Formula XI are used

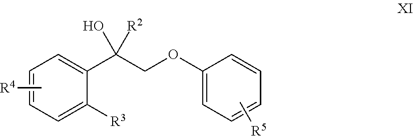

in which R², R³, R⁴ and R⁵ have the meanings designated above.

Compounds XI are alcohols obtained from the ketones of Formula 1X

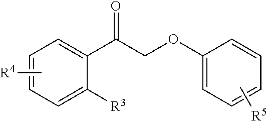

The R³ group represents in particular a hydrogen atom, an alkyl group, an alkoxy group, a halogen selected from fluorine, chlorine, bromine or iodine, and is in particular a hydrogen atom.

According to an advantageous embodiment of the invention, compounds of Formula XIA are used

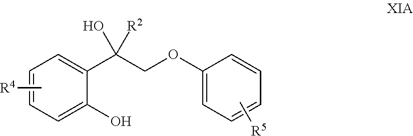

in which R², R⁴ and R⁵ have the meanings designated above.
These alcohols comprise an —OH in the ortho position on the —Ar group situated in α of carbon 1 of the formula

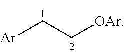

R⁴ may occupy the meta, para positions or the other ortho position of the ring; R⁵ may occupy the ortho, meta or para positions of the ring.

According to a particular aspect of the invention, compounds of Formulae XIIA, XIIB and XIIC are used

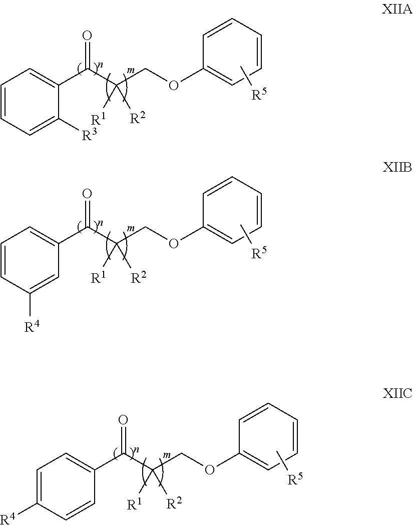

in which

R¹, R², R³, R⁴ and R⁵ have the meanings designated above,

n is equal to 0 or 1, m is equal to 0 or 1, provided that n+m=1.

The compounds of Formulae XIIA, XIIB and XIIC belong to the family of the 1-aryl-2-aryloxyethanes in which the aryl group in position 1 on the ethane chain of the following formula

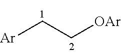

is monosubstituted.
In the compounds of Formula XIIA, the R³ group occupies the ortho position on the ring.
In the compounds of Formula XIIB, the R⁴ group occupies the meta position on the ring.
In the compounds of Formula XIIC, the R⁴ group occupies the para position on the ring.

According to another particular aspect of the invention, compounds of Formula 1 are used in which

R¹, R², R³, R⁴ and R⁵ have the meanings designated above, provided that R³ and R⁴ are both different from H,

n is equal to 0 or 1, m is equal to 0 or 1, provided that n+m=1.

These compounds belong to the family of the 1-aryl-2-aryloxyethanes in which the aryl group in position 1 on the ethane chain of the formula

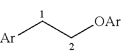

is disubstituted with the R³ and R⁴ groups, the meanings of which are defined above.

R³ is in the ortho position on the ring. R⁴ may occupy the meta, para position or the second ortho position of the ring. R³ and R⁴ are different from the hydrogen atom.

According to another particular aspect of the invention, compounds of Formulae XIIIA, XIIIB and XIIIC are used

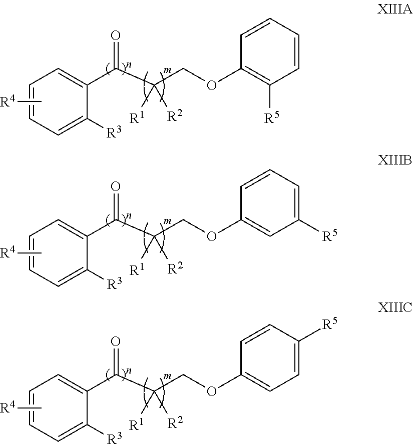

in which

R¹, R², R³, R⁴ and R⁵ have the meanings designated above,

n is equal to 0 or 1, m is equal to 0 or 1, provided that n+m=1.

The compounds of Formulae XIIIA, XIIIB and XIIIC belong to the family of the 1-aryl-2-aryloxyethanes in which the —OAr group in position 2 on the ethane chain of the formula

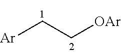

is substituted with the R⁵ group, the meanings of which are defined above.

R⁵ represents in particular a hydrogen atom, a halogen atom selected from fluorine, chlorine, bromine or iodine, —OH, an alkoxy group, an alkyl group optionally substituted with halogens, a nitrogen-containing group, and is in particular a hydrogen atom, a fluorine or bromine atom, —OH, —OCH₃, —O—CH₂—C₆H₅, —CH₃, —NO₂, —NH₂, —NH—C(O)—CH₃.

R⁵ may occupy all the positions on the ring:
In the compounds of Formula XIIIA, the R⁵ group occupies the ortho position,
In the compounds of Formula XIIIB, the R⁵ group occupies the meta position,
In the compounds of Formula XIIIC, the R⁵ group occupies the para position.

According to a particular aspect of the invention, compounds of Formulae XIID, XIIE and XIIF are used

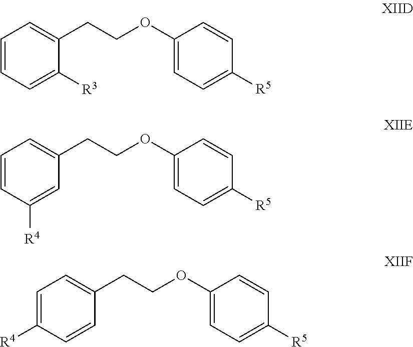

in which R³, R⁴ and R⁵ have the meanings designated above.
In the compounds of Formulae XIID, XIIE and XIIF, the R⁵ group is fixed in the para position.
The position of the group borne by the other aromatic ring, R³ or R⁴, is variable, all the positions being possible.
In the compounds of Formula XIID, the R³ group occupies the ortho position,
In the compounds of Formula XIIE, the R⁴ group occupies the meta position,
In the compounds of Formula XIIF, the R⁴ group occupies the para position.
   The invention relates to the use of the compounds of the following formulae:

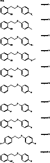

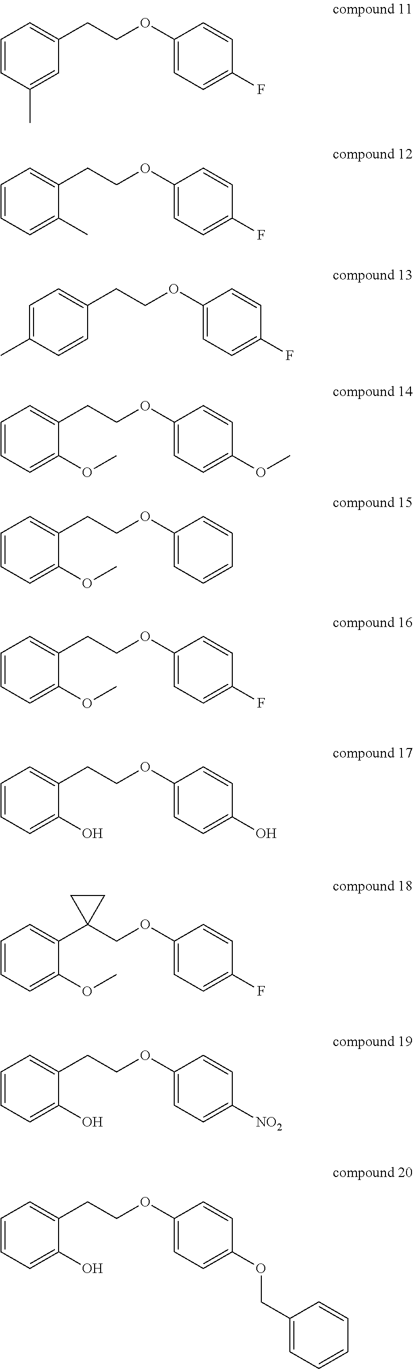

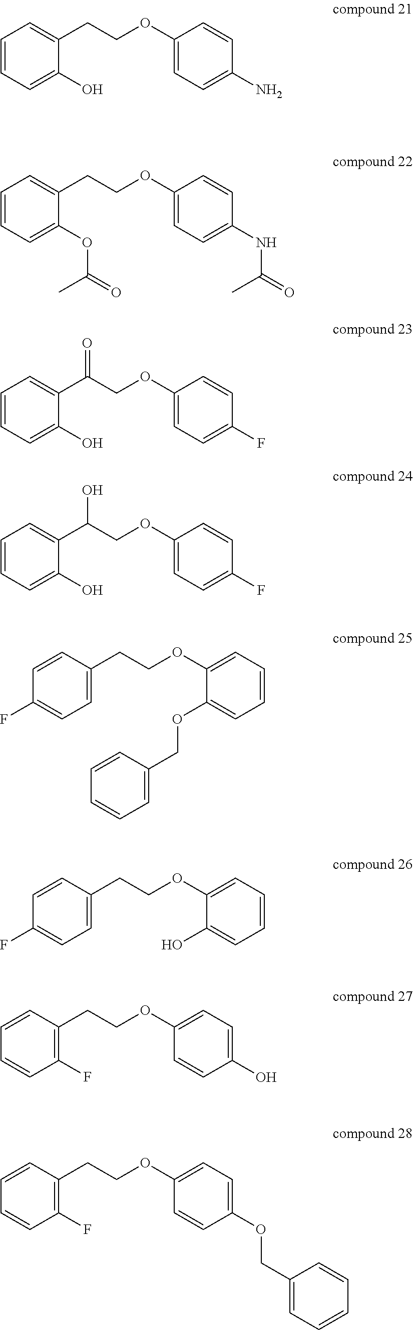

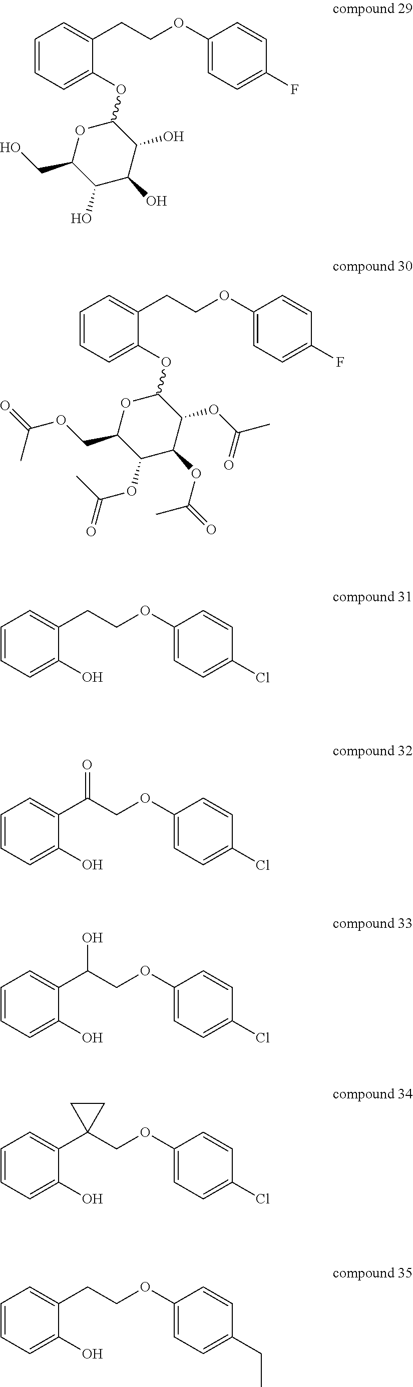

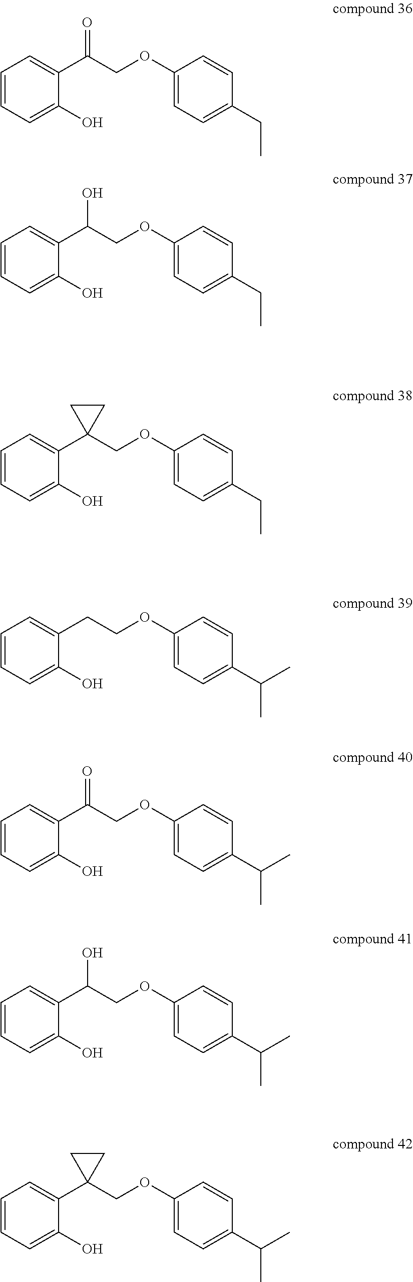

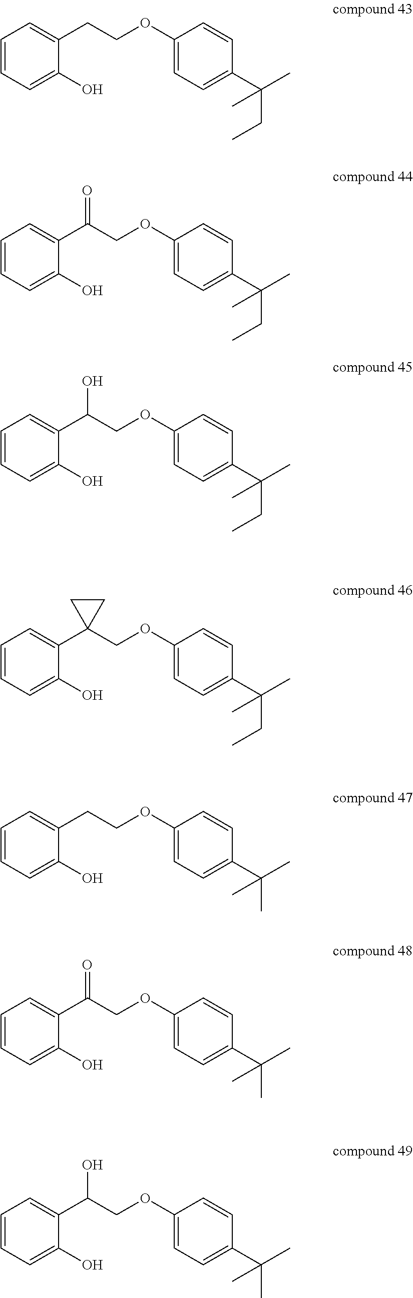

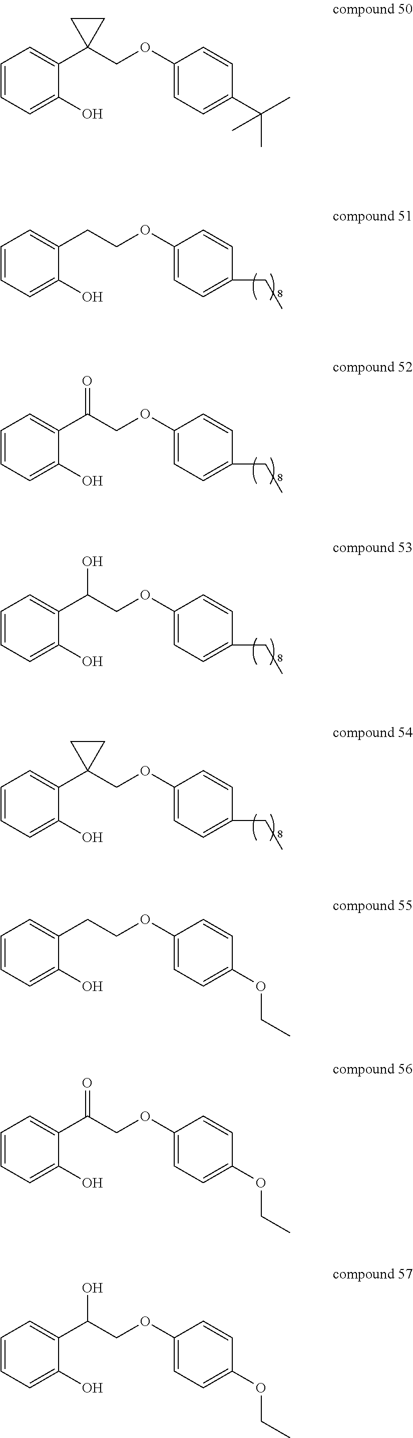

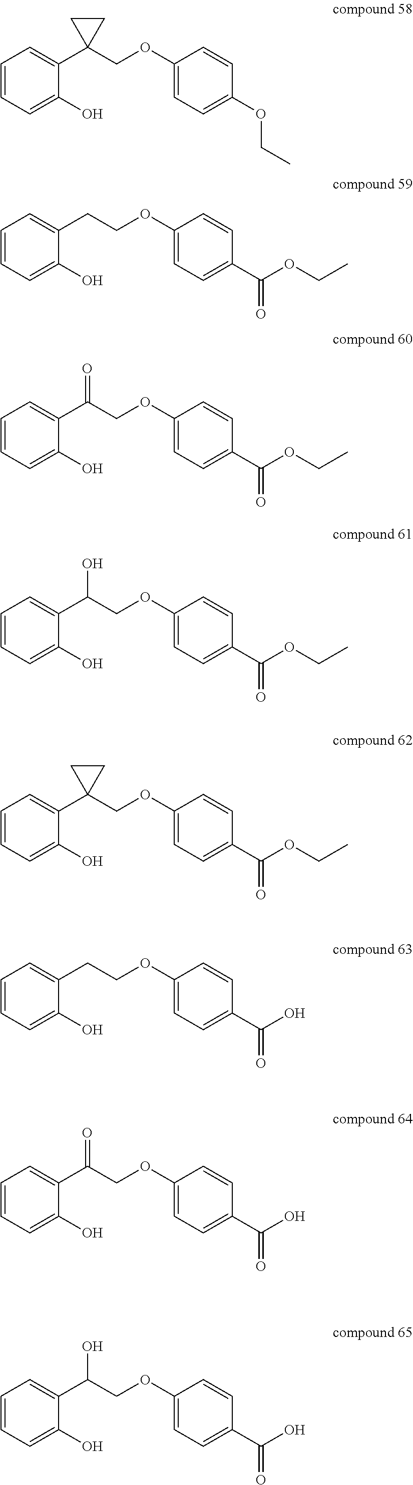

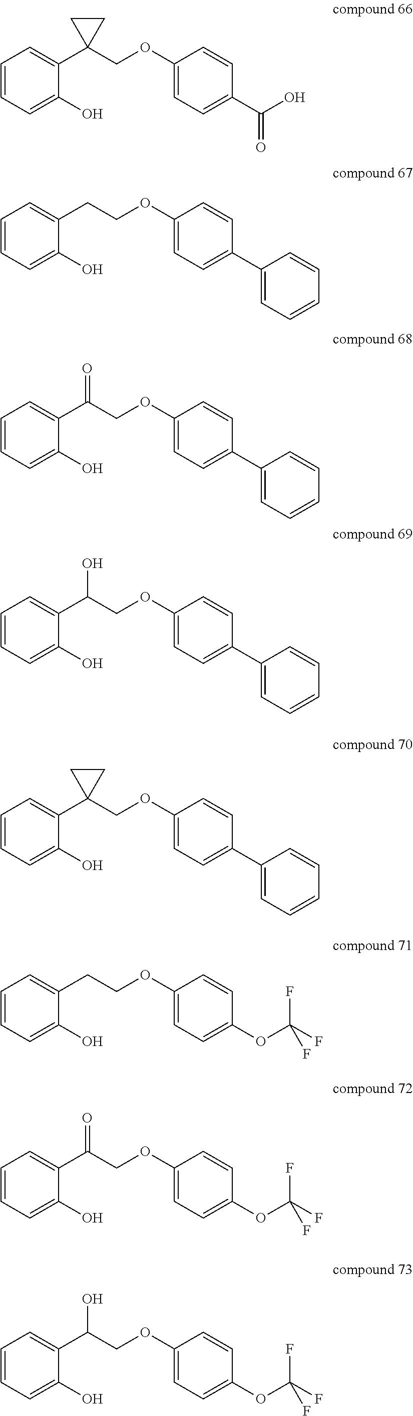

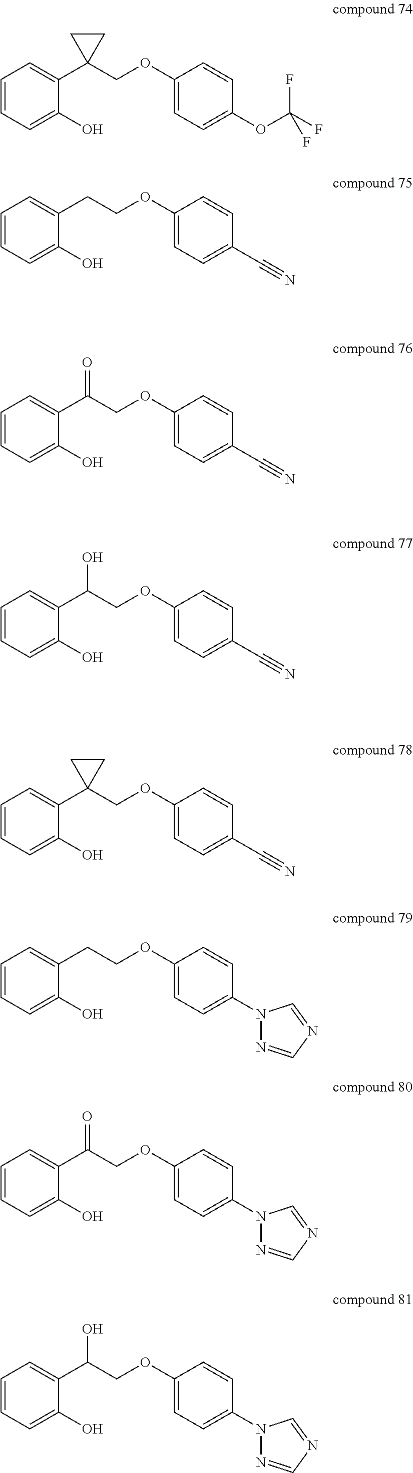

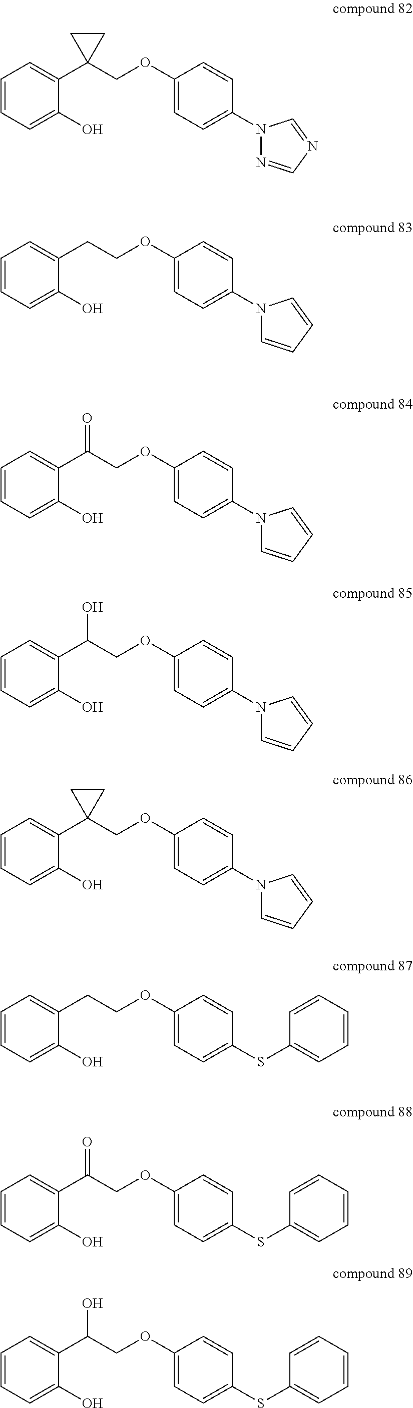

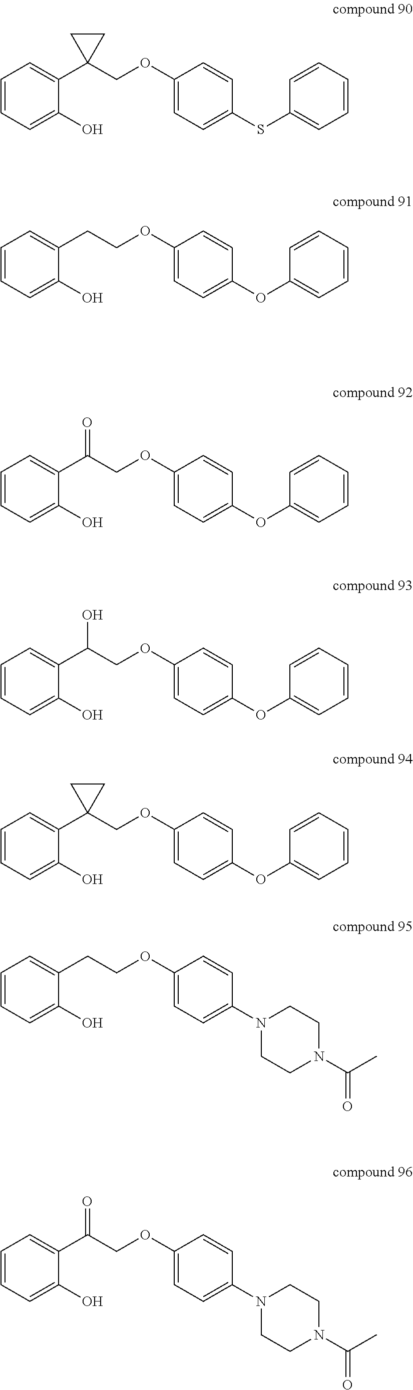

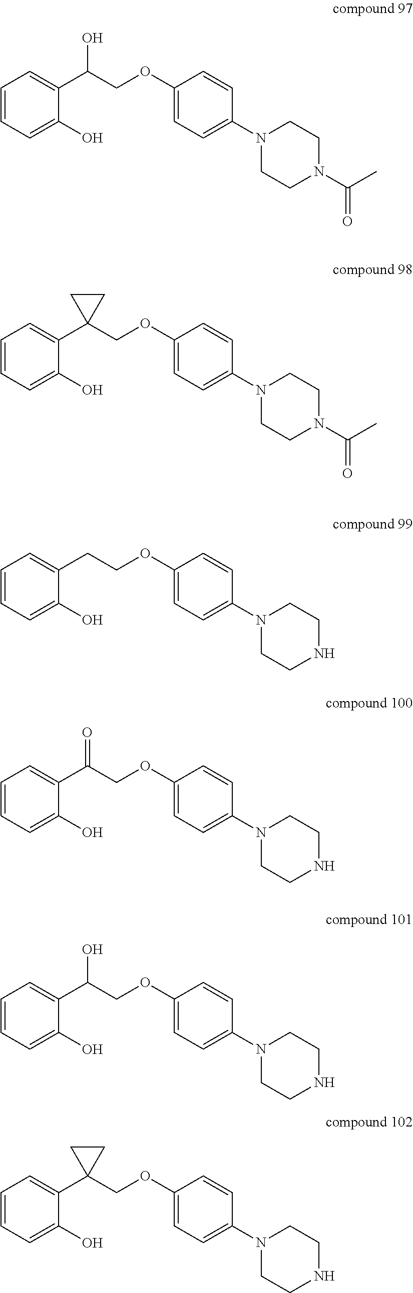 [SMILES: CC(=O)N1CCN(c2ccc(OCC(O)c3ccccc3O)cc2)CC1 ; CC(=O)N1CCN(c2ccc(OCC3(c4ccccc4O)CC3)cc2)CC1 ; Oc1ccccc1CCOc1ccc(N2CCNCC2)cc1 ; O=C(COc1ccc(N2CCNCC2)cc1)c1ccccc1O ; Oc1ccccc1C(O)COc1ccc(N2CCNCC2)cc1 ; Oc1ccccc1C1(COc2ccc(N3CCNCC3)cc2)CC1]

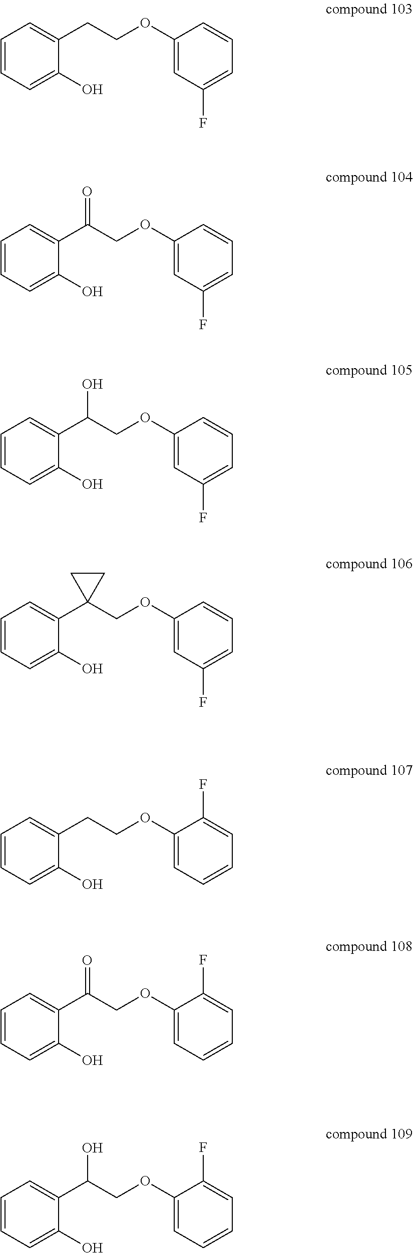

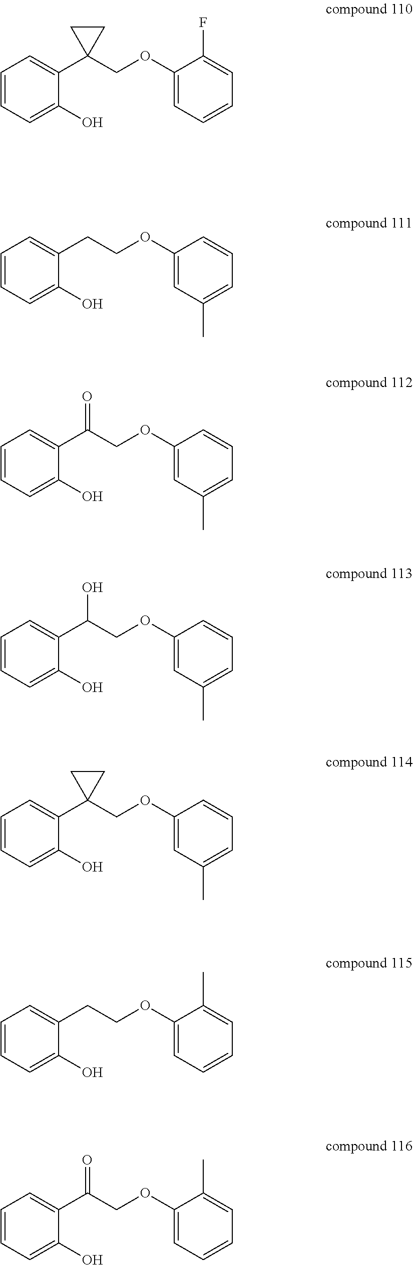

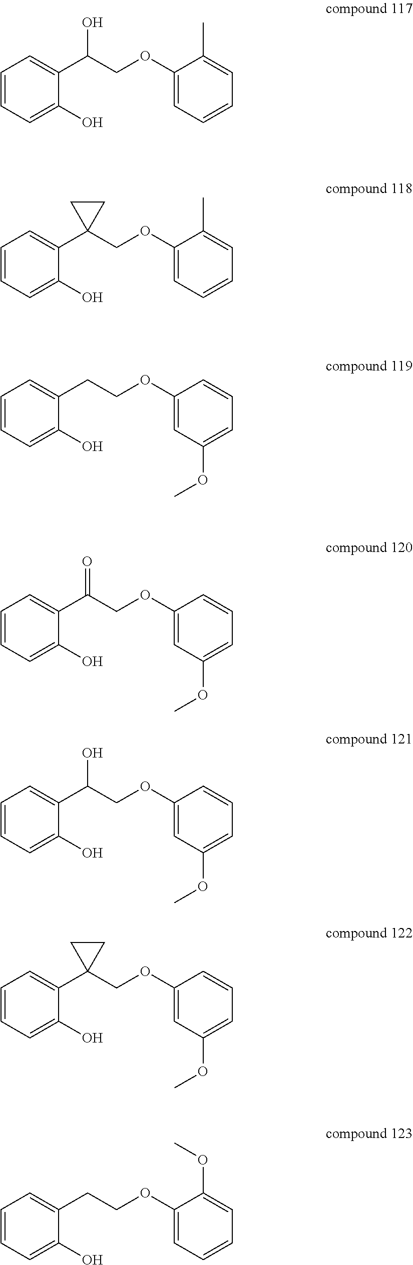

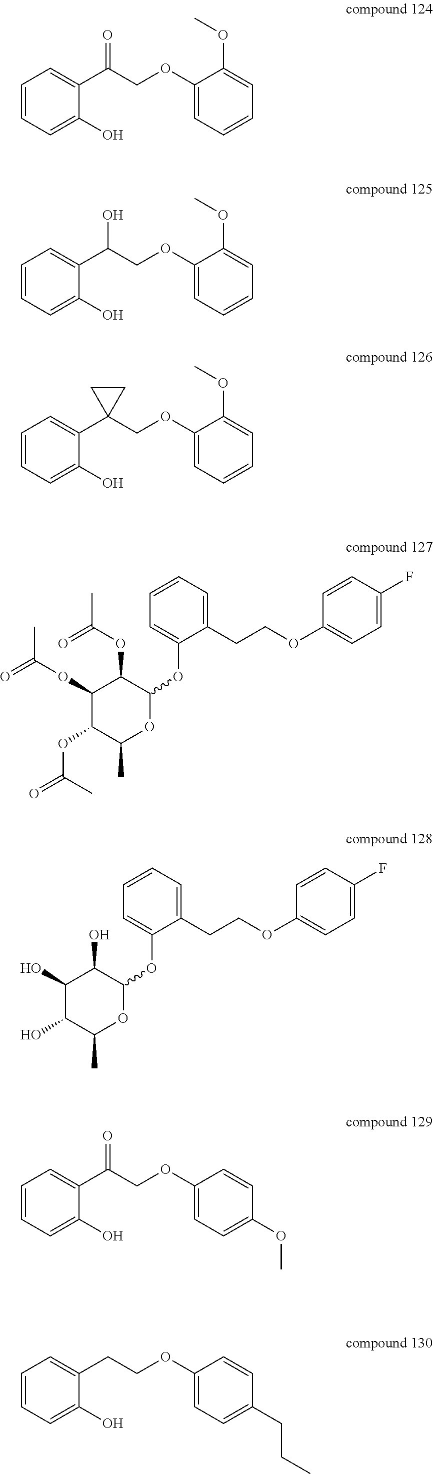

The invention also relates to the use of the compounds of the following formulae:

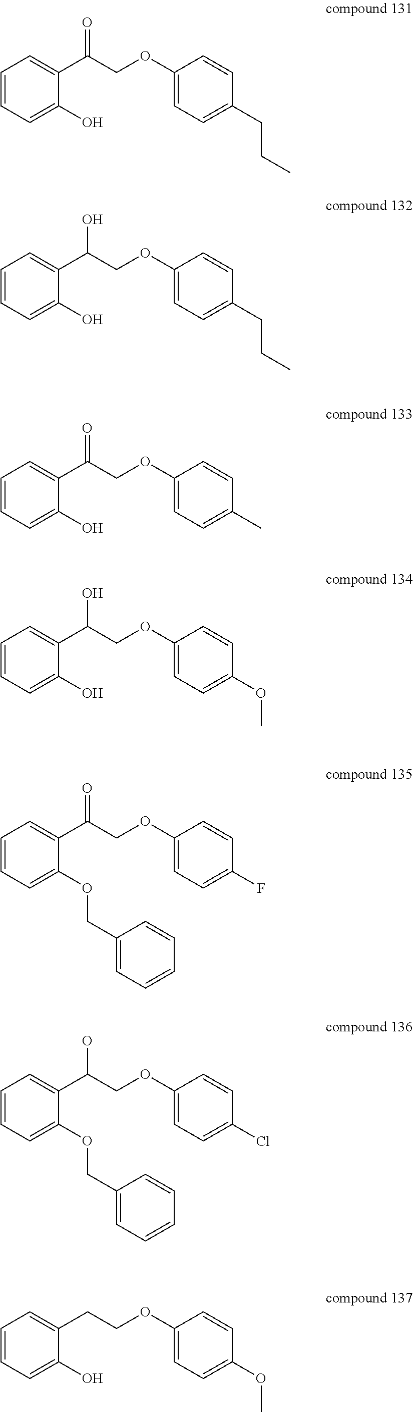

The compounds shown above are used as active ingredients for preparing cosmetic compositions having anti-ageing and/or depigmenting and/or wound-healing and/or anti-inflammatory properties.
The invention relates to the use of compounds 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, or 130 mentioned above for preparing cosmetic compositions having anti-ageing and/or depigmenting and/or wound-healing and/or anti-inflammatory properties.

The invention also relates to the use of the compounds 131, 132, 133, 134, 135, 136, or 137 mentioned above for preparing cosmetic compositions having anti-ageing and/or depigmenting and/or wound-healing and/or anti-inflammatory properties.

According to a particular aspect of the invention, one or more compounds of Formula 1 are used for preparing cosmetic compositions in which the anti-ageing properties belong to the group constituted by reinforcement of the dermis, proliferation of dermal fibroblasts, production of collagen, inhibition of expression of the “matrix metallopeptidase” gene MMP9 and of the protein “heat shock protein beta-1” HSPB1.

“Matrix metallopeptidase 9” (MMP-9) is an enzyme encoded by the MMP-9 gene. It is involved in the degradation of extracellular proteins. It is involved in the remodelling of the extracellular matrix and, in particular, in the degradation of collagens of types IV and V.

The “extracellular matrix” denotes all the extracellular macromolecules of connective tissue.

The protein “heat shock protein beta-1 HSPB1” plays a role in the regulation of cellular development, in the inhibition of apoptosis and, in particular, in cellular differentiation.

According to another particular aspect of the invention, one or more compounds of Formula 1 are used for preparing cosmetic compositions in which the anti-inflammatory properties belong to the group constituted by inhibition of leukocyte elastase or inhibition of secretion of prostaglandins E2.

The elastases are a sub-family of the serine proteases responsible for the degradation of elastin. The numerous natural substrates of this enzyme include, in addition to elastin, the proteoglycans of cartilage, fibronectin and the collagens of type I, II, III and IV. With respect to the skin, inhibition of elastase makes it possible to combat the effects of ageing, photo-induced or not, and limit the appearance of wrinkles and stretch marks.

The prostaglandins E2 (PGE2) play a role in inflammatory processes.

According to another particular aspect of the invention, one or more compounds of Formula 1 are used for preparing cosmetic compositions in which the wound-healing properties belong to the group constituted by the proliferation and migration of keratinocytes for covering a wound.

The phases of migration and proliferation of the cells are major phases of wound healing that occur after the inflammation phase. They are necessary for the recolonization of the wound. An increase in migration and proliferation of the cells allows wound healing to be improved.

The wound healing effect is evaluated:
by studying the proliferation and/or migration of fibroblasts,
by studying the proliferation and/or migration of keratinocytes.

In the study of the proliferation and/or migration of fibroblasts, the biological model is constituted by normal human dermal fibroblasts (NHDF), for which the culture conditions are 37° C. and 5% CO₂ in DMEM.

A control is carried out (NHDF in test medium DMEM 0% FCS) (n=6)) and a reference is used (FCS, Foetal Calf Serum at 10% (n=2)). For the compounds tested, n=2.

Incubation is for 72 hours.

The test consists of seeding normal human fibroblasts in 96-well plates suitable for migration studies. In these plates, the supports are pretreated with a solution of collagen and a mask is placed at the centre of each well, preventing adhesion of the cells in this zone and thus forming an artificial wound. After labelling the cells with calcein, the masks are removed and then the cells are treated with the compounds or the reference.

The migration of the cells is monitored microscopically for 72 hours, taking photographs at 0 h, 24 h, 48 h and 72 h. The results, expressed in percentage coverage, are compared with the untreated control.

In the study of the proliferation and/or migration of keratinocytes, the biological model is constituted by normal human epidermal keratinocytes (NHEK), for which the culture conditions are 37° C. and 5% CO₂ in a medium of keratinocytes-SFM-PE-EGF (keratinocyte culture medium—serum free medium (SFM) without pituitary extract (PE) and without Epidermal Growth Factor (EGF), control (n=6), reference: EGF (Epidermal Growth Factor) at 10 ng/ml (n=2), compounds tested (n=2).

Incubation is for 72 hours.

The test consists of seeding normal human keratinocytes in 96-well plates suitable for migration studies. In these plates, the supports are pretreated with a solution of collagen and a mask is placed at the centre of each well, preventing adhesion of the cells in this zone and thus forming an artificial wound. After labelling of the cells with calcein, the masks are removed and then the cells are treated with the compounds or the reference.

The migration of the cells is monitored microscopically for 72 hours, taking photographs at 0 h, 24 h, 48 h and 72 h. The results, expressed in percentage coverage, are compared with the untreated control.

According to another particular aspect of the invention, one or more compounds of Formula 1 are used for preparing cosmetic compositions in which the depigmenting properties belong to the group constituted by anti-tyrosinase activity and anti-melanogenesis activity.

“Anti-melanogenesis activity” makes it possible to decrease the production of melanin, the main pigment responsible for the colour of the skin.

As tyrosine is involved in the production of melanin, “anti-tyrosinase activity” allows inhibition of the production of melanin and, consequently, a depigmenting action.

The depigmenting effect was evaluated on a line of type B16 melanocytes stimulated with NDP-MSH, (natural hormone stimulating melanogenesis; Melanocyte Stimulating Hormone, [Nle, DPhe]-α-MSH). Melanin synthesis was evaluated.

The study was carried out for compounds in solution in DMSO. The effects of the following 3 compounds, at concentrations of 30 μmol·L⁻¹ (30 μM) and 100 μmol·L⁻¹ (100 μM), were thus compared:
compound of Formula 1, and in particular IVA1 (compound 1)
4-(2,4-dihydrophenyl)butane or rucinol [18979-61-8], already known for its depigmenting effects,
1-(2,4-dihydroxyphenyl)-3-(2,4-dimethoxy-3-methylphenyl)propane [869743-37-3].

The results obtained showed that the compounds of Formula 1 had a clear inhibitory effect. Moreover, a comparative study of the effects of the compounds of Formula 1 with the effects of the other two compounds mentioned above shows that the compounds of Formula 1 are more effective with respect to depigmentation than the other two compounds.

The invention relates to cosmetic compositions containing as active ingredient one or more compounds of Formula 1

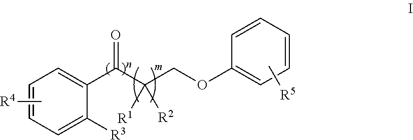

in which

R¹ and R², which may be identical or different, represent
hydrogen atoms,
halogen atoms selected from fluorine, chlorine, bromine or iodine,
linear or branched alkyl groups comprising from 1 to 10 carbon atoms, in particular the methyl, ethyl, isopropyl groups, optionally substituted with one or more halogens selected from fluorine, chlorine, bromine or iodine, with a —CF₃ group, with a hydroxy,
—OH groups,
linear or branched alkoxy groups comprising from 1 to 10 carbon atoms, in particular methoxy, ethoxy, optionally substituted with one or more halogens selected from fluorine, chlorine, bromine or iodine, and in particular the trifluoromethoxy group —OCF₃,
phenoxy groups —OPh,
aryloxy groups —OAr in which Ar represents an aromatic group comprising from 6 to 12 carbon atoms optionally substituted with one or more halogen atoms selected from fluorine, chlorine, bromine, or optionally substituted with one or more —OH groups in free or protected form, and in particular protected in the form of —OMes group, —OTHP group of formula

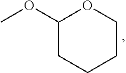

group derived from ethylene glycol of formula

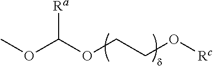

in which
δ varies from 1 to 12,
R^{a} represents a hydrogen or a linear or branched alkyl group comprising from 1 to 6 carbon atoms,
R^{c} represents a linear or branched alkyl group with 1 to 10 carbon atoms,
group derived from propylene glycol of formula,

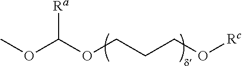

in which
δ′ varies from 1 to 5,
R^{a} and R^{c} have the meanings stated above,
group derived from a glycoside compound that may be α- or β-furanose or α- or β-pyranose, siloxy group of formula —OSi(R^{a})₃ in which R^{a} has the meanings stated above, —OSitBdPh group of formula

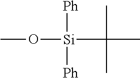 [SMILES: CO[Si](c1ccccc1)(c1ccccc1)C(C)(C)C]

—OSitBdM group of formula,

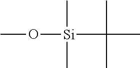

benzyloxy groups —OCH₂Ph,
alkenyl groups comprising from 2 to 10 carbon atoms, linear or branched and comprising at least one C═C double bond,
acyloxy groups comprising from 2 to 10 carbon atoms, derived from carboxylic acids,
—OTHP groups of the above formula,
—OMes groups,
groups derived from ethylene glycol of formula

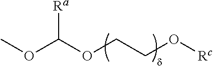

in which
δ varies from 1 to 12,
R^{a} represents a hydrogen or a linear or branched alkyl group comprising from 1 to 6 carbon atoms,
R^{c} represents a linear or branched alkyl group with 1 to 10 carbon atoms,
groups derived from propylene glycol of formula,

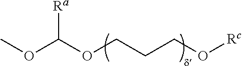

in which
δ′ varies from 1 to 5,
R^{a} and R^{c} have the meanings stated above,
—OH groups optionally coupled to glycoside compounds that may be α- or β-furanose or α- or β-pyranose,
siloxy groups of formula —OSi(R^{a})₃ in which R^{a} has the meanings stated above,
—OSitBdPh groups of formula

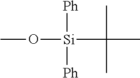 [SMILES: CO[Si](c1ccccc1)(c1ccccc1)C(C)(C)C]

—OSitBdM groups of formula,

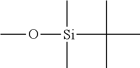

said R¹ and R² optionally forming a ring comprising 3 carbon atoms, said ring having the following formula

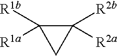

in which
R^{1a}, R^{1b}, R^{2a} and R^{2b}, which may be identical or different, represent
hydrogen atoms,
halogen atoms selected from fluorine, chlorine, bromine or iodine,
linear or branched alkyl groups comprising from 1 to 10 carbon atoms, in particular the methyl, ethyl, isopropyl groups, optionally substituted with one or more halogens selected from fluorine, chlorine, bromine or iodine, with a —CF₃ group, with a hydroxy, provided that R³ is a hydroxy group —OH,

R³ and R⁴, which may be identical or different, represent
hydrogen atoms,
halogen atoms selected from fluorine, chlorine, bromine,
linear, branched or cyclic alkyl groups comprising from 1 to 10 carbon atoms, in particular the methyl, ethyl, isopropyl groups, optionally substituted with one or more halogens selected from fluorine, chlorine, bromine or iodine, with a —CF₃ group, with a hydroxy,
hydroxyl groups —OH,
linear or branched alkoxy groups comprising from 1 to 10 carbon atoms, in particular the methoxy, ethoxy, isopropoxy, tert-butoxy groups, optionally substituted with one or more halogens selected from fluorine, chlorine, bromine or iodine, and in particular the trifluoromethoxy group —OCF₃,
benzyloxy groups,
an alkenyloxy group comprising from 2 to 10 carbon atoms, linear or branched and comprising at least one C═C double bond,
an alkynyloxy group comprising from 2 to 10 carbon atoms, linear or branched and comprising at least one C≡C triple bond,
—OTHP groups of formula

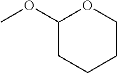

substituted —OTHP groups of the following formula

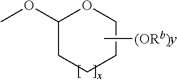

in which
x is equal to 0 or 1, y varies from 0 to 4,
R^{b} is a hydrogen or an acetyl —C(O)—CH₃,
groups derived from ethylene glycol of formula

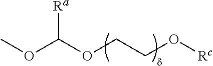

in which
δ varies from 1 to 12,
R^{a} represents a linear or branched alkyl group comprising from 1 to 6 carbon atoms,
R^{c} represents a linear or branched alkyl group with 1 to 10 carbon atoms,
groups derived from propylene glycol of formula

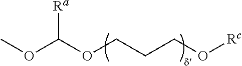

in which
δ′ varies from 1 to 5,
R^{a} and R^{c} have the meanings stated above,
—OH groups optionally coupled to a glycoside compound, which may be an α- or β-furanose or an α- or β-pyranose,
a siloxy group of formula —OSi(R^{a})₃ in which R^{a} has the meanings stated above,
—OSitBdPh of formula

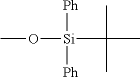 [SMILES: CO[Si](c1ccccc1)(c1ccccc1)C(C)(C)C]

—OSitBdM of formula,

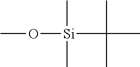

R⁵ represents
a hydrogen atom,
a halogen atom selected from fluorine, chlorine, bromine,
a linear, branched or cyclic alkyl group comprising from 1 to 10 carbon atoms, in particular a methyl, ethyl, isopropyl group, optionally substituted with one or more halogens selected from fluorine, chlorine, bromine or iodine, with a —CF₃ group, with a hydroxy,
an alkenyl group comprising from 2 to 6 carbon atoms, linear or branched and comprising at least one C═C double bond,
an alkynyl group comprising from 2 to 6 carbon atoms, linear or branched and comprising at least one C≡C triple bond,
a phenyl group optionally substituted with a halogen atom, a —CF₃ group, a linear or branched alkyl group comprising from 1 to 10 carbon atoms, a linear or branched alkoxy group comprising from 1 to 10 carbon atoms,
a hydroxyl group —OH,
a linear or branched alkoxy group comprising from 1 to 10 carbon atoms, in particular the methoxy, ethoxy, isopropoxy, tert-butoxy groups, optionally substituted with one or more halogens selected from fluorine, chlorine, bromine or iodine, and in particular the trifluoromethoxy group —OCF₃,
a benzyloxy group,
a phenoxy group,
a thiophenyl group,
an acyloxy group comprising from 2 to 10 carbon atoms, linear or branched,
an —OTHP group of formula

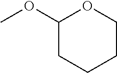

a substituted —OTHP group of the following formula

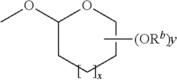

in which
x is equal to 0 or 1, y varies from 0 to 4,
R^{b} is a hydrogen or an acetyl —C(O)—CH₃,
an —OSO₂R^{c} group in which R^{c} is an alkyl group comprising from 1 to 6 carbon atoms, in particular methyl or ethyl, or an aromatic group comprising from 6 to 18 carbon atoms, optionally substituted with one or more halogen atoms, hydroxy or alkoxy group, nitro,
an —OSO₃M group in which M represents an Na⁺ or K⁺ ion,
a group derived from ethylene glycol of formula

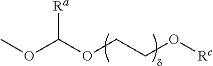

in which
δ varies from 1 to 12,
R^{a} represents a linear or branched alkyl group comprising from 1 to 6 carbon atoms,
R^{c} represents a linear or branched alkyl group with 1 to 10 carbon atoms,
a group derived from propylene glycol of formula

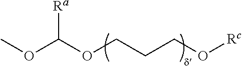

in which
δ′ varies from 1 to 5,
R^{a} and R^{c} have the meanings stated above,
an —OH group optionally coupled to a glycoside compound, which may be an α- or β-furanose or an α- or β-pyranose,
a siloxy group of formula —OSi(R^{a})₃ in which R^{a} has the meanings stated above,
—OSitBdPh of formula

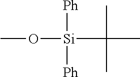 [SMILES: CO[Si](c1ccccc1)(c1ccccc1)C(C)(C)C]

—OSitBdM of formula,

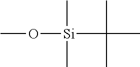

—COOH,
—CN,
—NH₂,
—NH₃⁺, X⁻
—NR^{d}R^{e},
—NHR^{d}R^{e+}, X⁻
—NHCOR^{f},
—NHCOOR^{g},
—NO₂,

R^{d}, R^{e} representing linear or branched alkyl groups comprising from 1 to 4 carbon atoms, optionally substituted with one or more halogen atoms, or carbon chains interrupted by oxygen or sulphur atoms, benzyl groups optionally substituted with a halogen atom, a hydroxy group, an alkoxy group comprising from 1 to 8 carbon atoms,

R^{f} represents a linear or branched alkyl group containing from 1 to 4 carbon atoms, a phthalimido group (in this case NH is replaced with N), a benzyl group optionally substituted with a halogen atom, a hydroxy group, an alkoxy group and in particular substituted with the methoxy group in the para position,

R^{g} represents a linear or branched alkyl group comprising from 1 to 4 carbon atoms, optionally substituted with one or more halogen atoms, or a carbon chain interrupted by oxygen or sulphur atoms, a phenyl group, a benzyl group optionally substituted with a halogen atom, a hydroxy group, an alkoxy group and in particular substituted with the methoxy group in the para position,

X⁻ representing a halide ion,
a group derived from piperazine, in particular

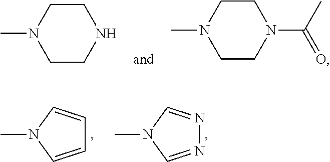

provided that R³ and R⁴ do not represent simultaneously —OH groups or groups comprising an oxygen atom bound to the phenyl,
said active ingredient being combined with a cosmetically acceptable vehicle.

According to a particular aspect, the invention relates to a cosmetic composition containing as active ingredient one or more compounds of Formula 1

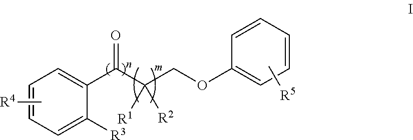

in which:

n is equal to 0 or 1, m is equal to 0 or 1, provided that n+m=1,

R¹ and R² have the meanings given above

R³ and R⁴, which may be identical or different, represent:
hydrogen atoms,
hydroxyl groups,

and R⁵ represents:
a linear, branched or cyclic alkyl group comprising from 2 to 10 carbon atoms, in particular an ethyl, isopropyl group, optionally substituted with one or more halogens selected from fluorine, chlorine, bromine or iodine, with a —CF₃ group, with a hydroxy,
an alkenyl group comprising from 2 to 6 carbon atoms, linear or branched and comprising at least one C═C double bond,
an alkynyl group comprising from 2 to 6 carbon atoms, linear or branched and comprising at least one C≡C triple bond.
   said active ingredient being combined with a cosmetically acceptable vehicle.

On account of their cosmetic properties, the compounds according to the invention are used therapeutically as skin depigmenting agents, anti-ageing, wound-healing, and anti-inflammatory agents.

For these purposes, they will be used in the form of cosmetic compositions containing as active ingredient at least one of the compounds of general formulae I in combination with or mixed with an excipient or an inert, non-toxic, cosmetically acceptable vehicle.

Excipients that are suitable for such administrations are oils, water and alcohol as well as surfactants, and additives such as preservatives, antioxidants, colorants, and perfumes.

The invention relates to cosmetic compositions constituted by a mixture comprising at least one compound of Formula 1 as active ingredient in combination with one or more compounds having anti-ageing and/or depigmenting and/or wound-healing and/or anti-inflammatory properties, in particular in combination with fatty acids such as linoleic acid or azelaic acid, and/or with antioxidants such as vitamin C and/or with tocopherol derivatives, and/or with desquamation additives such as retinoic acid or glycolic acid,

said composition having anti-ageing and/or depigmenting and/or wound-healing and/or anti-inflammatory properties.

The cosmetic compositions will be able to contain a mixture of compound of Formula 1 combined, in variable proportions, with one or more other compounds already known for their anti-ageing and/or depigmenting and/or wound-healing and/or anti-inflammatory and/or antioxidant properties.

The cosmetic compositions will thus optionally be able to contain another agent known for its depigmenting properties such as:
linoleic acid, deficiency of which leads to dry skin,
azelaic acid, a tyrosinase inhibitor, having a depigmenting effect on melasma, chloasma, and post-inflammatory hyperpigmentations,
retinoic acid, which is a tyrosinase inhibitor and also leads to moderate desquamation,
   or an antioxidant such as vitamin C or a tocopherol derivative.

According to a particular aspect of the invention, the cosmetic compositions have a content of compounds of Formula 1 from 0.001 to 10 wt %.

The dosage may vary depending on the form. If a mixture of compounds I is used, the proportions of compounds of Formula 1 in this mixture may vary, in such a way that the overall percentage by weight is from 0.001% to 10%.

According to a particular aspect of the invention, the cosmetic compositions are in the form of cream, ointment, gel, unguent, lotion, patch, oil, serum, milk, spray, balsam, emulsion, microemulsion.

The cosmetic compositions will be presented in one of the cosmetic forms suitable for cutaneous administration. In this respect, creams, ointments, gels, oils, serums, milks, sprays, emulsions, encapsulations may be mentioned.

According to a particular aspect of the invention, the cosmetic compositions contain as active ingredient one or more compounds of Formula II

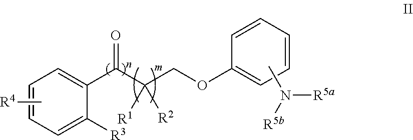

in which

n is equal to 0 or 1, m is equal to 0 or 1, provided that n+m=1,

R¹, R², R³ and R⁴ have the meanings designated above,

—NR^{5a}R^{5b} represents a group comprising a nitrogen atom fixed to the ring, optionally in the form of its salt, this group being selected from
—NH₂,
—NR^{d}R^{e},
—NHCOR^{f},
—NHCOOR^{g},
—NO₂,
a group derived from piperazine, in particular

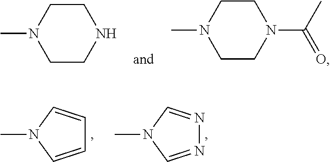

R^{d}, R^{e} representing linear or branched alkyl groups comprising from 1 to 4 carbon atoms, optionally substituted with one or more halogen atoms, or carbon chains interrupted by oxygen or sulphur atoms, benzyl groups optionally substituted with a halogen atom, a hydroxy group, an alkoxy group comprising from 1 to 8 carbon atoms,

R^{f} represents a linear or branched alkyl group containing from 1 to 4 carbon atoms, a phthalimido group (in this case NH is replaced with N), a benzyl group optionally substituted with a halogen atom, a hydroxy group, an alkoxy group and in particular substituted with the methoxy group in the para position,

R^{g} represents a linear or branched alkyl group comprising from 1 to 4 carbon atoms, optionally substituted with one or more halogen atoms, or a carbon chain interrupted by oxygen or sulphur atoms, a phenyl group, a benzyl group optionally substituted with a halogen atom, a hydroxy group, an alkoxy group and in particular substituted with the methoxy group in the para position,

in combination with a cosmetically acceptable vehicle.

These are compounds comprising a nitrogen-containing group, in particular nitro, amino, amido, borne by the —OAr group of the following general formula

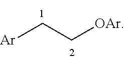

According to a particular aspect of the invention, the cosmetic compositions contain as active ingredient one or more compounds of Formula III

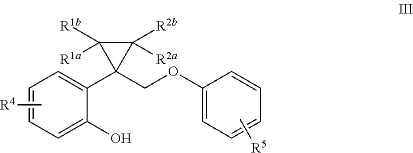

in which

R⁴ and R⁵ have the meanings designated above,

R^{1a}, R^{1b}, R^{2a} and R^{2b}, which may be identical or different, represent
hydrogen atoms,
halogen atoms selected from fluorine, chlorine, bromine or iodine,
linear or branched alkyl groups comprising from 1 to 10 carbon atoms, in particular the methyl, ethyl, isopropyl groups, optionally substituted with one or more halogens selected from fluorine, chlorine, bromine or iodine, with a —CF₃ group, with a hydroxy,
   in combination with a cosmetically acceptable vehicle.

These compounds are characterized in that they contain a cyclopropyl.

According to a particular aspect of the invention, the cosmetic compositions contain as active ingredient one or more compounds of Formula 1V

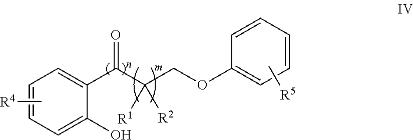

in which

R¹, R², R⁴ and R⁵ have the meanings designated above,

n is equal to 0 or 1, m is equal to 0 or 1, provided that n+m=1,

in combination with a cosmetically acceptable vehicle.

According to a particular aspect of the invention, the cosmetic compositions contain as active ingredient one or more compounds of Formula 1VA

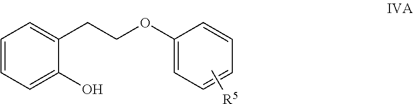

in which
R⁵ has the meaning designated above,
in combination with a cosmetically acceptable vehicle.

According to a particular aspect of the invention, the cosmetic compositions contain as active ingredient one or more compounds of Formula V

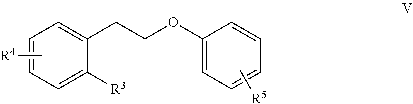

in which
R³, R⁴ and R⁵ have the meanings designated above,
provided that R³ and R⁴ are both different from —OH or are of any form in which —OH is protected,
in combination with a cosmetically acceptable vehicle.

According to a particular aspect of the invention, the cosmetic compositions contain as active ingredient one or more of the compounds of Formulae 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, or 130 mentioned above, in combination with a cosmetically acceptable vehicle.

According to a particular aspect of the invention, the cosmetic compositions contain as active ingredient one or more of the compounds of formulae 131, 132, 133, 134, 135, 136, or 137 mentioned above, in combination with a cosmetically acceptable vehicle.

According to a particular aspect of the invention, the cosmetic compositions contain a mixture comprising one or more compounds belonging to the list of compounds 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, or 130 mentioned above as active ingredient, in combination with one or more compounds having anti-ageing and/or depigmenting and/or wound-healing and/or anti-inflammatory properties, in particular in combination with fatty acids such as linoleic acid or azelaic acid, and/or with antioxidants such as vitamin C and/or with tocopherol derivatives, and/or with desquamation additives such as retinoic acid or glycolic acid, said composition having anti-ageing and/or depigmenting and/or wound-healing and/or anti-inflammatory properties.

According to a particular aspect of the invention, the cosmetic compositions contain a mixture comprising one or more compounds belonging to the list of compounds 131, 132, 133, 134, 135, 136, or 137 mentioned above as active ingredient, in combination with one or more compounds having anti-ageing and/or depigmenting and/or wound-healing and/or anti-inflammatory properties, in particular in combination with fatty acids such as linoleic acid or azelaic acid, and/or with antioxidants such as vitamin C and/or with tocopherol derivatives, and/or with desquamation additives such as retinoic acid or glycolic acid, said composition having anti-ageing and/or depigmenting and/or wound-healing and/or anti-inflammatory properties.

The invention also relates to the compounds of Formula 1

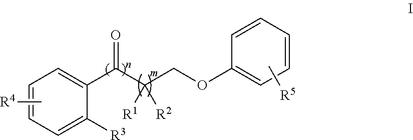

in which:

n is equal to 0 or 1, m is equal to 0 or 1, provided that n+m=1,

R¹ and R² have the meanings given above

R³ and R⁴, which may be identical or different, represent:
hydrogen atoms,
hydroxyl groups,

and R⁵ represents:
a linear, branched or cyclic alkyl group comprising from 2 to 10 carbon atoms, in particular an ethyl, isopropyl group, optionally substituted with one or more halogens selected from fluorine, chlorine, bromine or iodine, with a —CF₃ group, with a hydroxy,
an alkenyl group comprising from 2 to 6 carbon atoms, linear or branched and comprising at least one C═C double bond,
an alkynyl group comprising from 2 to 6 carbon atoms, linear or branched and comprising at least one C≡C triple bond.

The invention also relates to a compound of Formula X

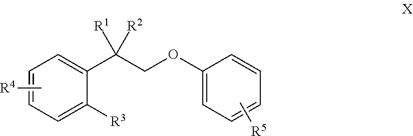

in which

n is equal to 0, and m is equal to 1,

R¹ and R² have the meanings given above,

R³ and R⁴, which may be identical or different, represent:
hydrogen atoms,
hydroxyl groups,

and R5 represents:
a linear, branched or cyclic alkyl group comprising from 2 to 10 carbon atoms, in particular an ethyl, isopropyl group, optionally substituted with one or more halogens selected from fluorine, chlorine, bromine or iodine, with a —CF₃ group, with a hydroxy,
an alkenyl group comprising from 2 to 6 carbon atoms, linear or branched and comprising at least one C═C double bond,
an alkynyl group comprising from 2 to 6 carbon atoms, linear or branched and comprising at least one C≡C triple bond.

The invention relates to the compounds of Formula II

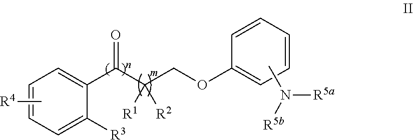

in which

n is equal to 0 or 1, m is equal to 0 or 1, provided that n+m=1,

R³ and R⁴ have the meanings designated above,

—NR^{5a}R^{5b} represents a group comprising a nitrogen atom fixed to the ring, optionally in the form of its salt, this group being selected from
—NH₂,
—NR^{d}R^{e},
—NHCOR^{f},
—NHCOOR^{g},
—NO₂,
a group derived from piperazine, in particular

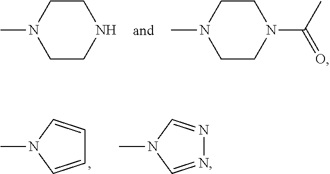

R^{d}, R^{e} representing linear or branched alkyl groups comprising from 1 to 4 carbon atoms, optionally substituted with one or more halogen atoms, or carbon chains interrupted by oxygen or sulphur atoms, benzyl groups optionally substituted with a halogen atom, a hydroxy group, an alkoxy group comprising from 1 to 8 carbon atoms,

R^{f} represents a linear or branched alkyl group containing from 1 to 4 carbon atoms, a phthalimido group (in this case NH is replaced with N), a benzyl group optionally substituted with a halogen atom, a hydroxy group, an alkoxy group and in particular substituted with the methoxy group in the para position,

R^{g} represents a linear or branched alkyl group comprising from 1 to 4 carbon atoms, optionally substituted with one or more halogen atoms, or a carbon chain interrupted by oxygen or sulphur atoms, a phenyl group, a benzyl group optionally substituted with a halogen atom, a hydroxy group, an alkoxy group and in particular substituted with the methoxy group in the para position.

These are compounds comprising a nitrogen-containing group, in particular nitro, amino, amido, borne by the —OAr group of the following general formula

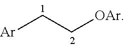

According to a particular aspect, the invention relates to compounds of Formula II

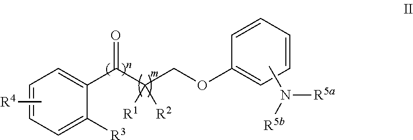

in which

n is equal to 0 or 1, m is equal to 0 or 1, provided that n+m=1,

R¹ and R² have the meanings designated above,

R³ and R⁴ have the meanings designated above, with the exception of the hydrogen atoms,

—NR^{5a}R^{5b} represents a group comprising a nitrogen atom fixed to the ring, optionally in the form of its salt, this group being selected from
—NH₂,
—NR^{d}R^{e},
—NHCOR^{f},
—NHCOOR^{g},
—NO₂,
a group derived from piperazine, in particular

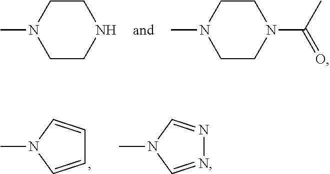

R^{d}, R^{e} representing linear or branched alkyl groups comprising from 1 to 4 carbon atoms, optionally substituted with one or more halogen atoms, or carbon chains interrupted by oxygen or sulphur atoms, benzyl groups optionally substituted with a halogen atom, a hydroxy group, an alkoxy group comprising from 1 to 8 carbon atoms,

R^{f} represents a linear or branched alkyl group containing from 1 to 4 carbon atoms, a phthalimido group (in this case NH is replaced with N), a benzyl group optionally substituted with a halogen atom, a hydroxy group, an alkoxy group and in particular substituted with the methoxy group in the para position,

R^{g} represents a linear or branched alkyl group comprising from 1 to 4 carbon atoms, optionally substituted with one or more halogen atoms, or a carbon chain interrupted by oxygen or sulphur atoms, a phenyl group, a benzyl group optionally substituted with a halogen atom, a hydroxy group, an alkoxy group and in particular substituted with the methoxy group in the para position.

These are compounds comprising a nitrogen-containing group, in particular nitro, amino, amido, borne by the —OAr group of the following general formula

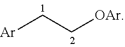

According to another particular aspect, the invention relates to compounds of Formula II

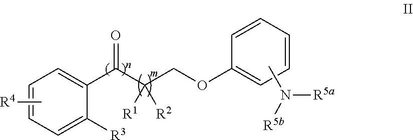

in which

n is equal to 0 or 1, m is equal to 0 or 1, provided that n+m=1,

R¹ and R² have the meanings designated above,

R³ represents a hydroxyl group and R⁴ has the meanings designated above,

—NR^{5a}R^{5b} represents a group comprising a nitrogen atom fixed to the ring, optionally in the form of its salt, this group being selected from
—NH₂,
—NR^{d}R^{e},
—NHCOR^{f},
—NHCOOR^{g},
—NO₂,
a group derived from piperazine, in particular

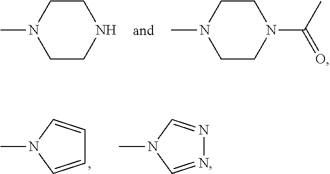

R^{d}, R^{e} representing linear or branched alkyl groups comprising from 1 to 4 carbon atoms, optionally substituted with one or more halogen atoms, or carbon chains interrupted by oxygen or sulphur atoms, benzyl groups optionally substituted with a halogen atom, a hydroxy group, an alkoxy group comprising from 1 to 8 carbon atoms,

R^{f} represents a linear or branched alkyl group containing from 1 to 4 carbon atoms, a phthalimido group (in this case NH is replaced with N), a benzyl group optionally substituted with a halogen atom, a hydroxy group, an alkoxy group and in particular substituted with the methoxy group in the para position,

R^{g} represents a linear or branched alkyl group comprising from 1 to 4 carbon atoms, optionally substituted with one or more halogen atoms, or a carbon chain interrupted by oxygen or sulphur atoms, a phenyl group, a benzyl group optionally substituted with a halogen atom, a hydroxy group, an alkoxy group and in particular substituted with the methoxy group in the para position.

These are compounds comprising a nitrogen-containing group, in particular nitro, amino, amido, borne by the —OAr group of the following general formula

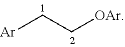

The invention relates to the compounds of Formula III

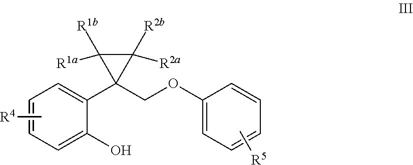

in which

R⁴ and R⁵ have the meanings designated above,

R^{1a}, R^{1b}, R^{2a} and R^{2b}, which may be identical or different, represent
hydrogen atoms,
halogen atoms selected from fluorine, chlorine, bromine or iodine,
linear or branched alkyl groups comprising from 1 to 10 carbon atoms, in particular the methyl, ethyl, isopropyl groups, optionally substituted with one or more halogens selected from fluorine, chlorine, bromine or iodine, with a —CF₃ group, with a hydroxy.

These compounds are characterized in that they contain a cyclopropyl.

The invention relates to the compounds of formulae

3, 7, 8, 9, 10, 11, 12, 13, 14, 18, 19, 20, 21, 22, 27, 28, 29, 30, 32, 33, 34, 35, 36, 37, 38, 39, 40, 41, 42, 43, 44, 45, 46, 47, 48, 49, 50, 51, 52, 53, 54, 55, 56, 57, 58, 60, 61, 62, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 120, 121, 122, 124, 125, 126, 127, 128, 129, or 130 mentioned above.

The invention also relates to the compounds of formulae 131, 132, 133, 134, 135, 136, or 137 mentioned above.

According to a particular aspect, the invention relates more particularly to the compounds of formula:

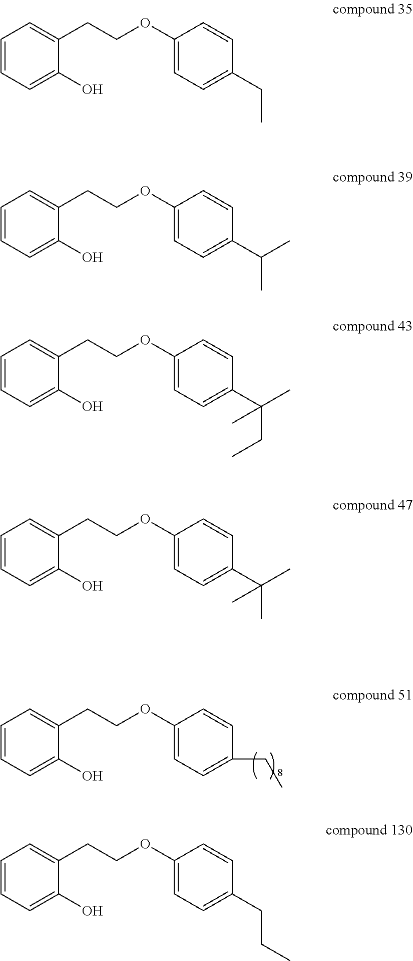

The invention relates to a process for preparing the compounds of Formula XA

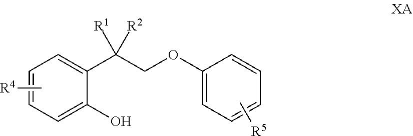

in which
R¹, R², R⁴ and R⁵ have the meanings designated above,

comprising a step of reaction of a phenolic alcohol

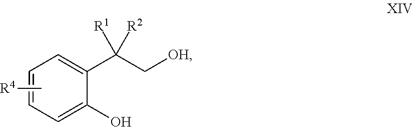

with mesyl chloride
in order to obtain the protected compound XV

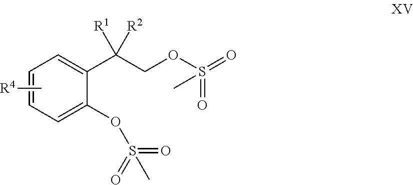

R¹, R², R⁴ having the meanings designated above,

comprising a step of reaction of compound XV of the above formula with a phenol derivative

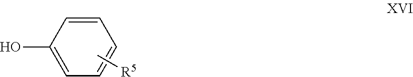

R⁵ having the meanings stated above,
in order to obtain the protected compound XVII,

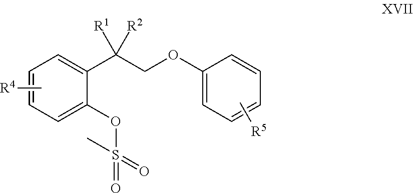

R¹, R², R⁴ and R⁵ having the meanings designated above,

comprising a step of deprotection of the phenolic function of compound XVII of the above formula

in order to obtain the compound of formula

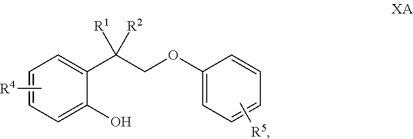

R¹, R², R⁴ and R⁵ having the meanings designated above,
and in particular R¹ and R² are both hydrogen atoms.

This process is applied for preparing compounds having an —OH group in the ortho position on the —Ar group of the following general formula

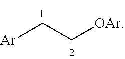

The synthesis, comprising 3 steps, is represented in the following diagram:

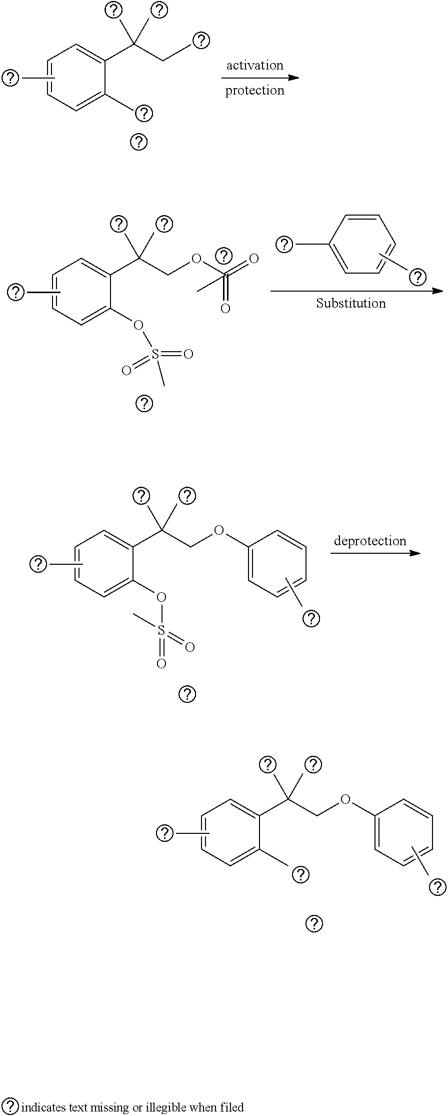

In the first step, the phenolic alcohol XIV reacts with mesyl chloride to give the dimesylated compound. This step constitutes a means of protecting the phenolic —OH on the one hand and of activating the —OH borne by the ethane chain on the other hand. The first step thus supplies the dimesyl compound XV which, in the second step, undergoes a substitution by reaction with the phenol XVI to give the compound XVII, belonging to the family of the 1-aryl-2-aryloxyethanes. Basic hydrolysis constitutes the last step and makes it possible to release the phenolic —OH from compound XA.

The invention also relates to a process for preparing the compounds of Formula V

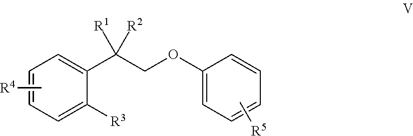

in which
R¹, R², R³, R⁴ and R⁵ have the meanings designated above,
provided that R³ and R⁴ are both different from —OH or are of any form in which —OH is protected,
comprising a reaction of the Mitsunobu type carried out on an aryl alcohol of formula

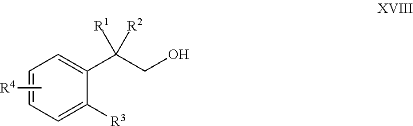

and a phenol XVI of formula

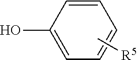

in order to obtain the compounds of Formula V,
R¹, R², R³, R⁴ and R⁵ have the meanings designated above,
and in particular R¹ and R² are both hydrogen atoms.

In contrast to the process described previously, this process is applied in order to prepare compounds that do not have an —OH group in the ortho position on the —Ar group of the following general formula

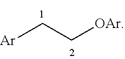

The synthesis, consisting of carrying out a Mitsunobu reaction, is represented in the following diagram:

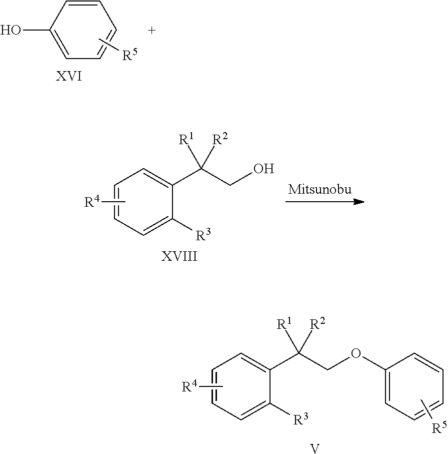

Compound XVIII reacts with the phenol XVI in the presence of triphenylphosphine and then diethyl azodicarboxylate to give a compound of the family of the 1-aryl-2-aryloxyethanes, R³ being different from —OH or from any protected form of —OH.

The invention also relates to a process for preparing the compounds of Formula III

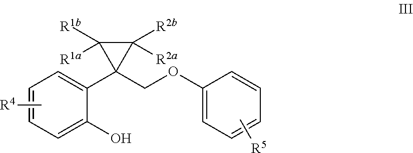

R⁴ and R⁵ having the meanings designated above,

R^{1a}, R^{1b}, R^{2a} and R^{2b}, which may be identical or different, representing
hydrogen atoms,
halogen atoms selected from fluorine, chlorine, bromine or iodine,
linear or branched alkyl groups comprising from 1 to 10 carbon atoms, in particular the methyl, ethyl, isopropyl groups, optionally substituted with one or more halogens selected from fluorine, chlorine, bromine or iodine, with a —CF₃ group, with a hydroxy,

said process comprising a step of reaction of the Mitsunobu type on an alcohol of formula

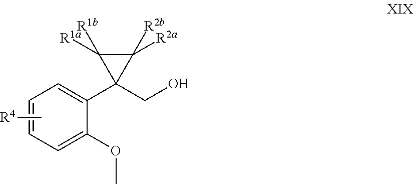

R⁴, R^{1a}, R^{1b}, R^{2a} et R^{2b} having the meanings designated above,
in order to obtain compound XX

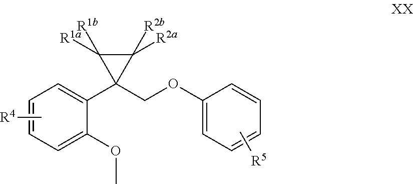

R⁵ having the meanings designated above,

said process comprising a reaction of deprotection of the phenolic —OH of compound XX shown above,

in order to obtain compound III

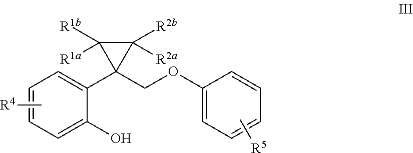

R^{1a}, R^{1b}, R^{2a}, R^{2b}, R⁴ and R⁵ having the meanings designated above, and in particular R^{1a}, R^{1b}, R^{2a} and R^{2b} being four hydrogen atoms.

In this process, the Mitsunobu reaction is carried out on the cyclopropanoic alcohol XIX and is followed by deprotection of the phenolic —OH, as shown in the following diagram:

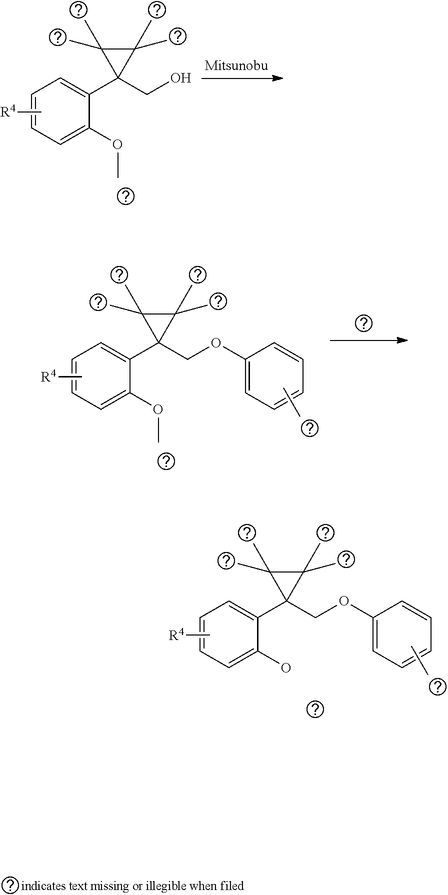

When R^{1a}═R^{1b}═R^{2a}═R^{2b}═R⁴═H, compound XIX is commercially available.

When R^{1a}═R^{1b}═R^{2a}═R^{2b}═H and R⁴ and R⁵ have the meanings designated above, the derivatives may be obtained by formation of the cyclopropyl by the techniques known to a person skilled in the art (Cy C et al., Eur. J. Med. Chem 1991 26(2) p 125-128; Kirmse W et al., Chem. Ber. 1986 119(2) p 3694-3703; Qiao J et al., Bioorg. Med. Chem. Lett 2009 19(2) p 462-468): for example dibromoethane under basic conditions (Muthusamy S et al., Tet. Lett. 2005 46(4) p 635-638).

The conditions for deprotection make use of the knowledge of a person skilled in the art and of conventional techniques such as the use of boron tribromide.

The invention also relates to a process for preparing the compounds of Formula IIA

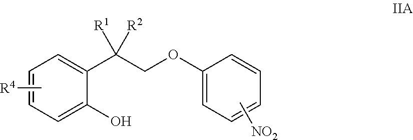

in which R¹, R² and R⁴ have the meanings designated above,

comprising a step of reaction of a phenolic alcohol

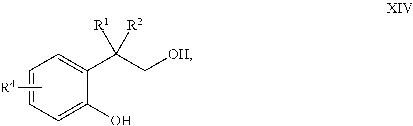

with mesyl chloride
in order to obtain the protected compound XV

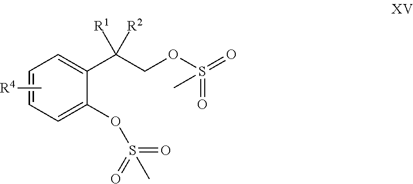

R¹, R² and R⁴ having the meanings designated above,

comprising a step of reaction of compound XV of the above formula with a phenol derivative

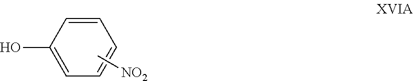

in order to obtain the protected compound XVIIA

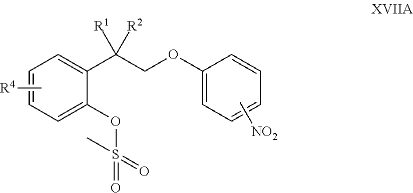

R¹, R² and R⁴ having the meanings designated above,

comprising a step of deprotection of the phenolic function of compound XVIIA of the above formula

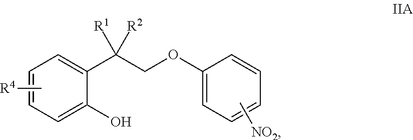

in order to obtain the compound of formula
R¹, R² and R⁴ having the meanings designated above,
and in particular R¹ and R² being two hydrogen atoms.

The invention also relates to a process for preparing the compounds of Formula IIB

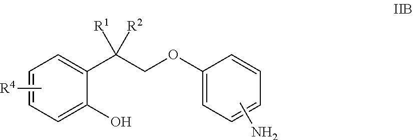

R¹, R² and R⁴ having the meanings designated above,
comprising a reduction of the nitro compound IIA of formula

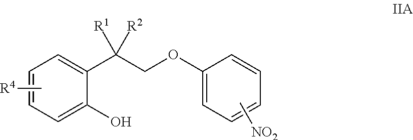

by reduction under pressure of hydrogen in the presence of a catalyst
in order to obtain compound IIB shown above.

The invention relates to a process for preparing the compounds of Formula IIC

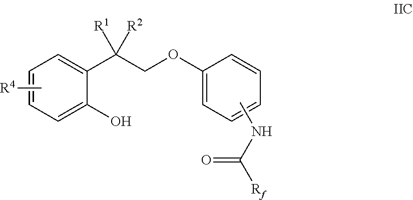

in which
R¹, R², R⁴ and R_{f} have the meanings designated above,

comprising a step of reaction of a phenolic alcohol

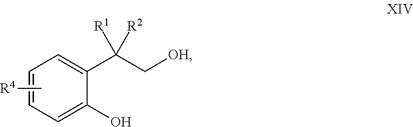

with mesyl chloride
in order to obtain the protected compound XV

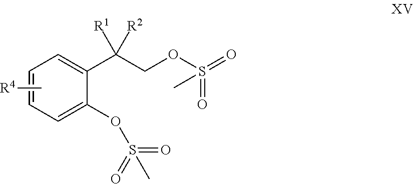

R¹, R², R⁴ having the meanings designated above,

comprising a step of reaction of compound XV of the above formula with a phenol derivative

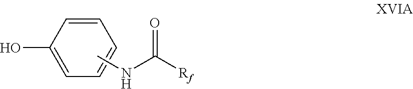

in order to obtain the protected compound XVIIA,

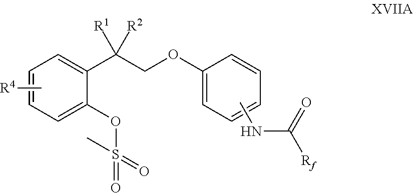

R¹, R², R⁴ and R_{f} having the meanings designated above,

comprising a step of deprotection of the phenolic function of compound XVIIA of the above formula

in order to obtain the compound of Formula IIC,

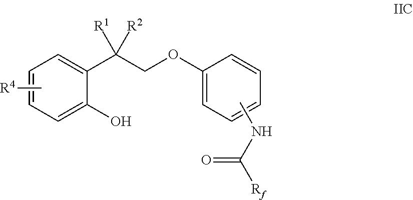

R¹, R², R⁴ and R_{f} having the meanings designated above,
and in particular R¹ and R² are both hydrogen atoms.

This route makes it possible to obtain the compounds bearing an amide function.

The acetamidophenol of Formula XVIA with R_{f}═—CH₃ is commercially available. The molecules of Formula XVIA may be obtained by direct amidation of aminophenol with an anhydride or chloride acid derivative, as described by Fiez-David in Helv. Chim. Acta 1939, 22 p 89, Palm K et al., J. Med. Chem. 1998, 41(27), 5382-5392, Deng W et al., Bioorg. Med. Chem. Lett. 2006, 16(2), 469-472.

The invention relates to a process for preparing the compounds of Formula 1

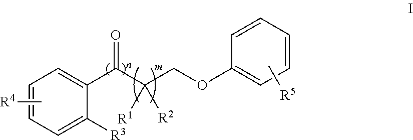

in which

n, m, R¹, R², R⁴ and R⁵ have the meanings designated above,

R³ is an —OH group coupled to a glycoside compound, which may be an α- or β-furanose or an α- or β-pyranose,

said process comprising a coupling reaction between compound IV of formula

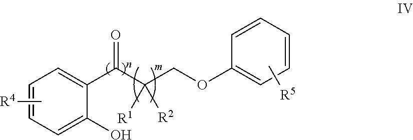

and a sugar, which may be an α- or β-furanose or an α- or β-pyranose, the —OH being protected, in particular in the form of acetates, and the anomeric position being activated beforehand in the form of bromine or of trichoroacetimidate,
in order to give a sugar derivative of Formula 1 shown above.

This is a modification of the Kœnigs-Knorr type. The phenol function of the compound of general formula I reacts with a glycoside unit, which may be an α- or β-furanose or an α- or β-pyranose, protected and previously activated in the form of brominated derivative, which may be commercially available, or in the form of trichloroacetimidate derivative. The preparation of the latter is described in the literature (Thisbe K. Lindhorst, Essentials of Carbohydrate Chemistry and Biochemistry, WILEY-VCH 2000). This coupling may or may not be followed by deprotection of the acetates, for example in a basic medium.

### EXAMPLES

### Meaning of the Abbreviations Used

TEA: triethylamine

DMSO: dimethylsulphoxide

NDP-MSH: [Nle, DPhe]-α-MSH

DMEM: Dulbecco/Vogt modified Eagle's minimal essential medium (medium for cell culture developed by Harry Eagle; it is used for maintaining the cells of a tissue culture).

The analytical techniques are as follows:

### Nuclear Magnetic Resonance:

The NMR spectra were recorded at 300 MHz (Brucker spectrometer) for the proton. The chemical shifts are expressed in ppm, the residual chloroform being taken as internal reference (singlet at 7.28 ppm), or the residual dimethylsulphoxide being taken as internal reference (multiplet at 2.50 ppm). The multiplicity of the signals is denoted by the following letters: s singlet, d doublet, dd doublet of doublets, t triplet, q quadruplet and m multiplet.

### Chromatography: LCMS

LC/MS analysis corresponds to coupling of HPLC analysis and analysis by mass spectrometry. It is carried out on an Alliance Waters 2695-ZQ2000 instrument.

### HPLC (Waters ref. 2690)

Detector: DAD detector (Waters, ref.: 2996, λ=190 nm to 800 nm):

### Detector: Corona™ (ESA):

Mass detector (Waters, ref. ZQ2000): 100-1500 dalton; negative and positive ion
Temperature of HPLC furnace: 40° C.
Flow rate: 1 mL/min

The methods used for HPLC are shown below. The method and the retention time are stated for the analytical results.

XTerra method
Column: XTerra® MS C18: 4.6 mm×150 mm, 5 nm (Waters, ref. 186000490)
Eluent A: Water (HCOOH-0.02%); Eluent B=CH₃CN) with elution gradient
Elution conditions: gradient

| | HCO₂H at | | |
|---|---|---|---|
| Min. | 0.2‰ | MeCN | Curve |
| | 90 | 10 | |
| 4 | 75 | 25 | 8 |
| 5 | 65 | 35 | 6 |
| 11 | 5 | 95 | 7 |
| 14 | 5 | 95 | 7 |
| 17 | 90 | 10 | 6 |
| 20 | 90 | 10 | 6 |

### XBridge Method

Column: XBridge™ C18: 4.6 mm×150 mm, 5 nm (Waters, ref. 186003116)
Eluent A: Water (HCOOH-0.02%); Eluent B=CH₃CN) with elution gradient
Elution conditions: gradient

| | HCO₂H at | | |
|---|---|---|---|
| Min. | 0.2‰ | MeCN | Curve |
| | 90 | 10 | |
| 4 | 75 | 25 | 8 |
| 5 | 65 | 35 | 6 |
| 11 | 5 | 95 | 7 |
| 14 | 5 | 95 | 7 |
| 17 | 90 | 10 | 6 |
| 20 | 90 | 10 | 6 |

### Example 1

### General Procedure for Preparing the Compounds of Formula XA

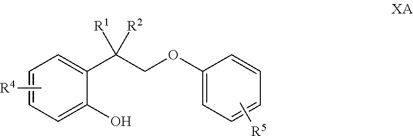

R¹, R², R⁴ and R⁵ having the meanings designated above,
by a multi-step synthesis represented in the following diagram:

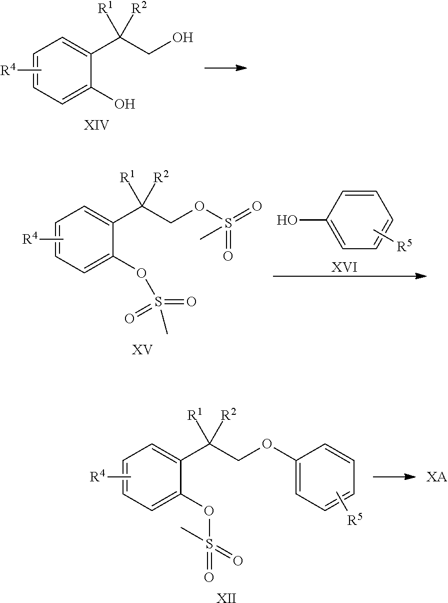

Step 1: protection/activation

In a dry 250 mL three-necked flask, under a nitrogen stream and with magnetic stiffing, the diol XIV (1 equivalent) is diluted in dichloromethane (5 volumes) and then TEA (2.2 equivalents) is added. The reaction medium is cooled to 5° C. Using a dropping funnel, methanesulphonyl chloride (2.2 equivalents), diluted in dichloromethane (5 volumes) is added while controlling the temperature. The reaction medium is then stirred at ambient temperature overnight. The reaction medium is poured into an ice water/HCl mixture. After extracting with dichloromethane three times, the organic phases are washed with saturated NaCl solution. After drying over MgSO₄ and filtering on a frit, the filtrate is concentrated under vacuum. The residue is purified by trituration in pentane. The products obtained are oils or solids depending on the nature of R¹, R² and R⁴.
Step 2: substitution

In a dry 100 mL three-necked flask, under a nitrogen stream and with magnetic stirring, the dimesylate compound XV (1 equivalent) is dissolved in acetonitrile (10 volumes). A phenol of Formula XVI (1.2 equivalents) is added, as well as K₂CO₃ (1.2 equivalents). The reaction mixture is heated under reflux for 4 h. At ambient temperature, the reaction medium is filtered to remove the salts and the filtrate is concentrated under vacuum. The residue is taken up in dichloromethane and an aqueous solution of NaHCO₃. After extracting with dichloromethane three times, the organic phases are washed with saturated NaCl solution. After drying over MgSO₄, the organic phase is filtered and concentrated under vacuum. The products obtained are monomesylates of Formula XII, oils or solids depending on the nature of R¹, R², R⁴ and R⁵, purified in particular by silica chromatography or by trituration in isopropyl ether/pentane mixtures of variable composition.
Step 3: deprotection

In a 50 mL three-necked flask equipped with a condenser, with magnetic stiffing, the monomesylate of Formula XII (1 equivalent) is dissolved in ethanol (12 volumes). A soda solution at 2 mol·L⁻¹ (2 equivalents) is added and the mixture is heated with stiffing under reflux for 4 h. The reaction medium is concentrated under vacuum at ambient temperature. The residue is taken up in water and then the solution is acidified. It is extracted with dichloromethane and the organic phases are washed with saturated NaCl solution. After drying over MgSO₄, the organic phase is filtered and concentrated under vacuum. The compounds XA are obtained, oils or solids, purified in particular by silica gel chromatography.

### Example 2

### Preparation of Compound 1 of Formula

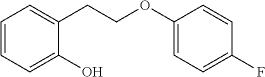 [SMILES: Oc1ccccc1CCOc1ccc(F)cc1]

This is prepared by the multi-step synthesis represented in the following diagram:

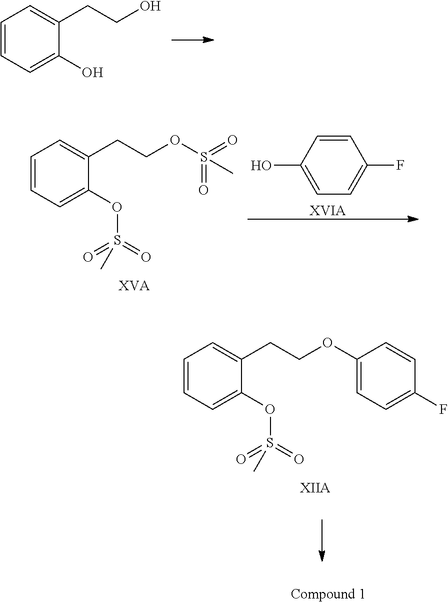

Step 1: protection/activation

Step 1 is carried out according to the procedure described in Example 1 starting from compound XIVA. After filtration and drying under vacuum, compound XVA is isolated. It is a white solid obtained with 92% yield. It is used in the next step.
Step 2: substitution

Step 2 is carried out according to the procedure described in Example 1 using 4-fluorophenol. The residue is purified on a silica gel column eluted with a heptane—ethyl acetate gradient. The monomesylate XIIA is obtained. This is a yellow oil, obtained with 55-65% yield. It is used in the next step.
Step 3: deprotection

Step 3 is carried out according to the procedure described in Example 1. The residue is purified on a silica gel column eluted with a heptane—ethyl acetate gradient. Compound 1 is a colourless oil obtained with 60-70% yield.

¹H NMR (300 MHz, CDCl₃): δ (ppm) 3.14 (t, 2H, CH₂—C₆H₅); 4.24 (t, 2H, CH₂—O); 6.81 (s, 1H, —OH); 6.85-7.22 (m, 8H, H aromatic).

HPLC: XTerra tᵣ=12.25 min

ES⁻ mass: [M−H]⁻=231.2.

### Example 3

### General Procedure for Preparing Compounds V; Mitsunobu Reaction for the Molecules without Free Phenol Function

The synthesis is represented in the following diagram:

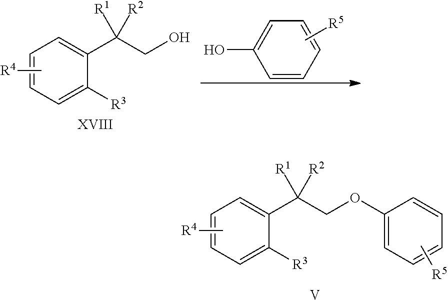

R¹, R², R³, R⁴ and R⁵ having the meanings designated above.

General procedure: In a 25 mL three-necked flask, under a nitrogen stream and with magnetic stirring, the alcohol compound XVIII (1 equivalent) is diluted in tetrahydrofuran (16 volumes) and then a phenol derivative (1 equivalent) and triphenylphosphine (1.3 equivalents) are added. The reaction medium is stirred at ambient temperature. Then diethyl azodicarboxylate (1.3 equivalents) is added dropwise. The reaction medium is stirred at ambient temperature overnight. The reaction medium is concentrated. The residue is taken up in distilled water and ethyl acetate. After extraction with ethyl acetate three times, the organic phases are washed with saturated NaCl solution. After drying over MgSO₄ and filtering on a frit, the filtrate is concentrated under vacuum. The residue is purified by silica gel chromatography eluted with a heptane/ethyl acetate gradient.

### Example 4

### Mitsunobu Reaction for the Molecules without Free Phenol Function

The phenol derivative may in particular be 4-fluorophenol.

The synthesis with 4-fluorophenol is represented in the following diagram:

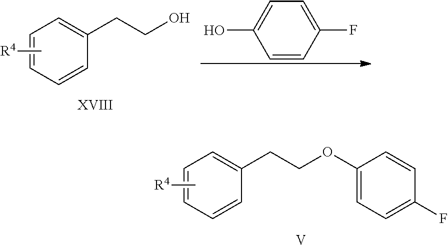

Procedure: in the case where R⁴ is a methyl in the para position

In a 25 mL three-necked flask, under a nitrogen stream and with magnetic stiffing, compound XVIII (1 equivalent) is diluted in tetrahydrofuran (15 volumes) and then 4-fluorophenol (1 equivalent) and triphenylphosphine (1.3 equivalents) are added. The reaction medium is stirred at ambient temperature. Then diethyl azodicarboxylate (1.3 equivalents) is added dropwise. The reaction mixture is stirred at ambient temperature overnight. The reaction medium is concentrated. The residue is taken up in distilled water and ethyl acetate. After extraction with ethyl acetate three times, the organic phases are washed with saturated NaCl solution. After drying over MgSO₄ and filtering on a frit, the filtrate is concentrated under vacuum. The residue is purified by silica gel chromatography eluted with a heptane/ethyl acetate gradient. The fraction containing the desired product is concentrated: a colourless oil is obtained with 54% yield.

¹H NMR (300 MHz, CDCl₃): δ (ppm): 2.34 (s, 3H, —CH₃); 3.09 (t, 2H, CH₂—C₆H₅); 4.15 (t, 2H, CH₂—O); 6.85-7.29 (m, 8H, aromatic H).

HPLC: XTerra tᵣ=13.56 min

ES⁺ mass: [M+Na]⁺=253.3.

### Example 5

### General Procedure for Preparing Compounds IIB; Reduction Reaction for the Molecules with a Free Amine Function

The synthesis is represented in the following diagram:

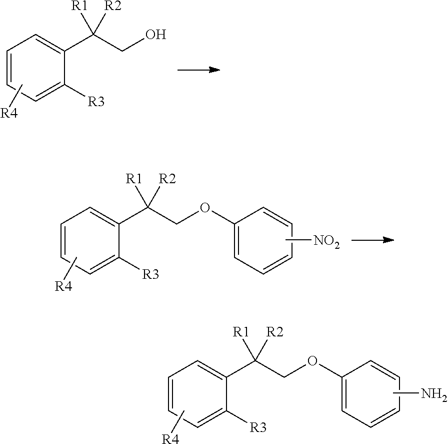

R¹, R², R³ and R⁴ having the meanings designated above.

General procedure for the step of reduction of the nitro function: in a flask equipped with a three-way cock, compound IIA (1 equivalent) is diluted in ethyl acetate (20 volumes) and then the reaction medium is purged by alternation of vacuum/nitrogen atmosphere. The 10% palladium on charcoal catalyst is added and the reaction medium is purged by alternation of nitrogen atmosphere and hydrogen atmosphere. The reaction medium is stirred at ambient temperature under atmospheric pressure of hydrogen for 7 h. After filtration on Celite cake, the filtrate is concentrated under vacuum and then the residue is purified by silica chromatography or by crystallization.

### Example 6

### Reduction Reaction for the Molecules with Free Amine Function

The derivative XVIII may in particular be 2-hydroxyphenylethanol.

The Synthesis is in this Case:

This reduction step is carried out according to the procedure described in Example 5 starting from compound 19, itself obtained according to the general procedure described in Example 1.

Compound 21 is obtained, after crystallization from dichloromethane, in the form of a beige solid, at 60% yield.

¹H NMR (300 MHz, DMSO-d6): δ (ppm) 2.90 (t, 2H, CH₂—C₆H₅); 3.96 (t, 2H, CH₂—O); 4.58 (bs, 2H, —NH₂); 6.46-7.14 (m, 8H, aromatic H); 9.42 (bs, 1H, —OH).

HPLC: XTerra tᵣ=7.63 min

ES⁺ mass: [M+H]⁺=230.3.

### Example 7

### Coupling Reaction of a Glycoside Derivative on a Phenol Function

The general synthesis is represented in the following diagram:

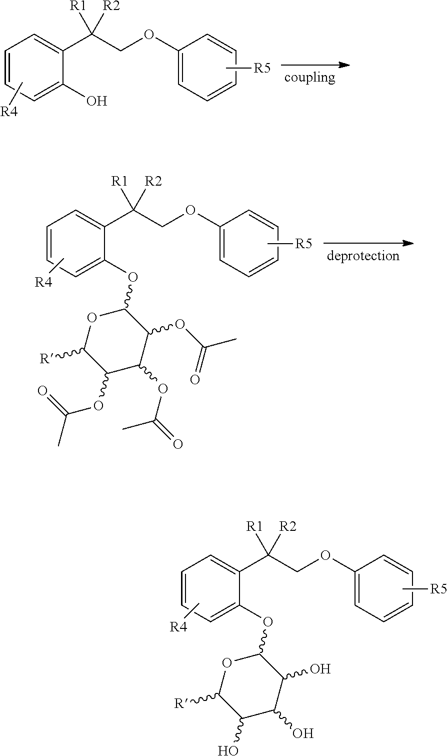

R¹, R², R⁴ and R⁵ have the meanings designated above.
Step 1: coupling of the activated glycoside derivative in the form of trichloroacetimidate

In a 50 mL three-necked flask, under a nitrogen stream and with magnetic stirring, the phenol derivative of Formula V (1 equivalent) is dissolved in dichloromethane (7 volumes), then the activated glycoside derivative in the form of trichloroacetimidate (1.3 equivalents) is introduced, dissolved beforehand in 8 volumes of dichloromethane. At 0° C., in the presence of beads of molecular sieve, a solution of BF₃.Et₂O (2 equivalents) is added. The reaction medium is stirred for 2 h at ambient temperature. It is then poured into a saturated solution of NaHCO₃. After extraction with dichloromethane, the organic phases are washed with saturated NaCl solution. After drying over MgSO₄ and filtering on a frit, the filtrate is concentrated under vacuum. The residue is purified by silica gel chromatography eluted with a heptane/dichloromethane gradient. The fraction containing the desired product is concentrated.
Step 2: deprotection of the hydroxyls of the glycoside unit

In a 20 mL flask, under inert atmosphere, the preceding derivative (1 equivalent) is dissolved in methanol (10 volumes). At ambient temperature, a 1M solution of sodium methanolate is added and the reaction medium is stirred for 2.5 h. After adding acid resin, the medium is filtered and concentrated under vacuum. The residue obtained is purified by chromatography or by trituration in carefully selected solvents.

### Example 8

### Preparation of Compound 29

The general synthesis is represented in the following diagram:

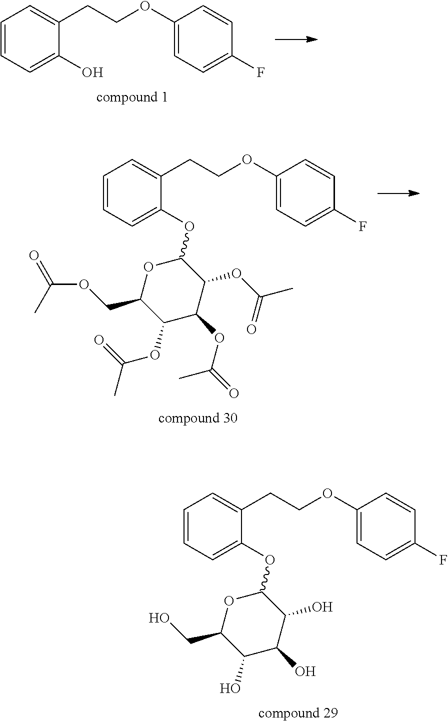 [SMILES: Oc1ccccc1CCOc1ccc(F)cc1 ; CC(=O)OC[C@H]1OC(Oc2ccccc2CCOc2ccc(F)cc2)[C@H](OC(C)=O)[C@@H](OC(C)=O)[C@@H]1OC(C)=O ; OC[C@H]1OC(Oc2ccccc2CCOc2ccc(F)cc2)[C@H](O)[C@@H](O)[C@@H]1O]

Step 1: Coupling of the activated glycoside derivative

This Step 1 is carried out according to the procedure described in Example 7 starting from compound 1. After purification by silica gel chromatography, compound 30 is isolated. It is a white solid obtained with 85% yield.

¹H NMR (300 MHz, DMSO-d6): δ (ppm) 2.05 (s, 12H, —CO—CH₃); 3.08 (t, 2H, CH₂—C₆H₅); 3.90 (m, 1H, anomeric H); 4.02-4.33 (m, 4H, glucose H); 5.10-5.23 (m, 2H, glucose H); 5.35 (t, 2H, CH₂—O); 6.80-7.29 (m, 8H, aromatic H).

HPLC: XBridge tᵣ=13.16 min

ES⁻ mass: [M+H₂O—H]⁻=580.2.
Step 2: deprotection

Step 2 is carried out according to the procedure described in Example 7 starting from compound 30. After purification by trituration in a pentane/isopropyl ether mixture, compound 29 is isolated. It is a white solid obtained with 80% yield.

¹H NMR (300 MHz, DMSO-d6): δ (ppm) 3.04 (m, 2H); 3.12 (m, 1H); 3.28 (t, 2H, CH₂—C₆H₅); 3.46 (m, 1H); 3.83 (m, 1H); 4.16 (t, 2H, CH₂—O); 4.59 (m, 1H, —OH); 4.83 (d, 1H); 5.04-5.32 (m, 3H, —OH); 6.93-7.25 (m, 8H, aromatic H).

HPLC: XBridge tᵣ=10.10 min

ES⁻ mass: [M−H]⁻=393.4.

### Example 9

### General Procedure for Preparing Compounds 35, 39, 43, 47, 51 and 130

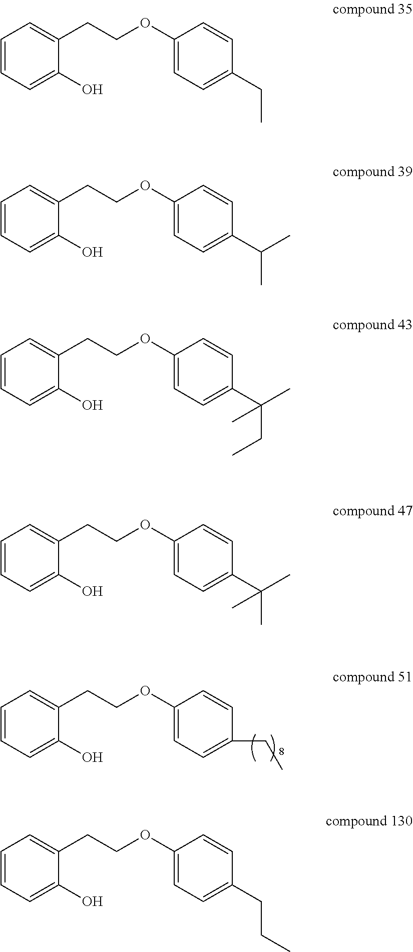

A multi-step synthesis, represented in the following diagram, is carried out:

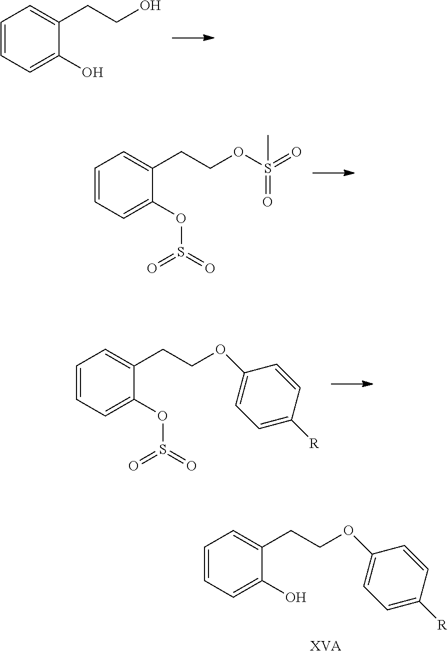

Step 1: protection/activation

In a dry 250 mL three-necked flask, under a nitrogen stream and with magnetic stirring, the compound 2-(2-hydroxy-ethyl)-phenol (1 equivalent) is diluted in dichloromethane (5 volumes) and then TEA (2.2 equivalents) is added. The reaction medium is cooled to 5° C. Using a dropping funnel, methanesulphonyl chloride (2.2 equivalents), diluted in dichloromethane (5 volumes) is added while controlling the temperature. The reaction medium is then stirred at ambient temperature overnight. The reaction medium is poured into an ice water/HCl mixture. After extracting with dichloromethane three times, the organic phases are washed with saturated NaCl solution. After drying over MgSO₄ and filtering on a frit, the filtrate is concentrated under vacuum. The residue is purified by trituration in pentane. The products obtained are oils or solids depending on the nature of R.
Step 2: substitution

In a dry 100 mL three-necked flask, under a nitrogen stream and with magnetic stiffing, compound XVA (1 equivalent) is dissolved in acetonitrile (10 volumes).

Phenol derivatives (1.2 equivalents) are added, as well as K₂CO₃ (1.2 equivalents).

Said phenol derivatives are as follows:

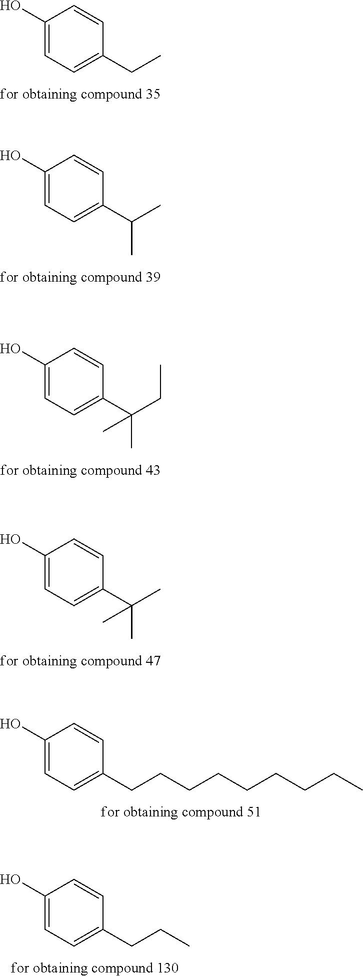

The reaction medium is heated under reflux for 4 h. At the end of the reaction, the reaction medium is hydrolysed with 25 volumes of distilled water. After extracting with dichloromethane twice, the organic phases are washed with a saturated solution of NH₄Cl, dried over MgSO₄, filtered and concentrated under vacuum.

The residue obtained is purified by silica gel chromatography eluted with a heptane/chloroform or heptane/isopropyl ether or heptane/isopropanol gradient, depending on the polarity of the compounds.

The colourless liquids, obtained with 20-60% yield, are each used in the next step of deprotection.
Step 3: deprotection

In a 50 mL three-necked flask equipped with a condenser, with magnetic stiffing, the monomesylate of formula

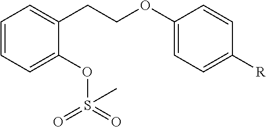

(1 equivalent) is dissolved in ethanol (12 volumes). A soda solution at 2 mol·L⁻¹ (2 equivalents) is added and the mixture is heated with stiffing under reflux for 4 h.

Once the reaction has ended after 20 h, the solvent ethanol is removed under vacuum and the residue is taken up in a mixture of distilled water and dichloromethane. After extracting repeatedly, the organic phases are dried over MgSO₄, filtered and concentrated under vacuum. The residue is purified by silica gel chromatography eluted with a heptane/ethyl acetate gradient.

The yield of the reaction is 45-70%.

The compounds obtained are characterized as follows:

Compound 35: colourless oil

¹H NMR (300 MHz, CDCl₃): δ (ppm) 1.22 (t, 3H, CH₃); 2.60 (q, 2H, CH₂—CH₃); 3.13 (t, 2H, CH₂-Ph); 4.26 (t, 2H, CH₂—O); 6.84-6.96 (m, 4H, aromatic H); 7.10-7.21 (m, 4H, aromatic H)

HPLC: XBridge tᵣ=13.2 min

ES⁺ mass: [M+H]⁺=243.1

Compound 39: colourless oil

¹H NMR (300 MHz, CDCl₃): δ (ppm) 1.22 (s, 6H, CH₃); 2.87 (heptuplet, 1H, CH—); 3.13 (t, 2H, CH₂-Ph); 4.26 (t, 2H, CH₂—O); 6.85-6.95 (m, 4H, aromatic H); 7.13-7.21 (m, 4H, aromatic H)

HPLC: XBridge tᵣ=13.5 min

ES⁺ mass: [M+H]⁺=255.2

Compound 43: colourless oil

¹H NMR (300 MHz, CDCl₃): δ (ppm) 0.66 (t, 3H, —CH₂—CH₃); 1.26 (s, 6H, —C—CH₃); 1.62 (m, 2H, —C—CH₂—); 3.13 (t, 2H, CH₂-Ph); 4.27 (t, 2H, CH₂—O); 6.85-7.26 (m, 8H, aromatic H)

HPLC: XBridge tᵣ=13.9 min

ES⁻ mass: [M−H]⁻=283.2

Compound 47: colourless oil

¹H NMR (300 MHz, CDCl₃): δ (ppm) 1.30 (t, 9H, —C—(CH₃)₃); 3.13 (t, 2H, CH₂-Ph); 4.27 (t, 2H, CH₂—O); 6.86-7.33 (m, 8H, aromatic H)

HPLC: XBridge tᵣ=13.7 min

ES⁻ mass: [M−H]⁻=269.2

Compound 51: colourless oil

¹H NMR (300 MHz, CDCl₃): δ (ppm) 0.46-1.67 (m, 19H, —(CH₂)₈—CH₃); 3.13 (t, 2H, CH₂-Ph); 4.27 (t, 2H, CH₂—O); 6.84-6.96 (m, 4H, aromatic H); 7.13-7.24 (m, 4H, aromatic H)

HPLC: XBridge tᵣ=12.6 min

ES⁻ mass: [M−H]⁻=339.3

Compound 130: colourless oil

¹H NMR (300 MHz, CDCl₃): δ (ppm) 0.93 (t, 3H, —CH₂—CH₃); 1.60 (m, 2H, —CH₂—CH₃); 2.54 (t, 2H, —CH₂—CH₂—); 3.14 (t, 2H, CH₂-Ph); 4.26 (t, 2H, CH₂—O); 6.84-6.97 (m, 4H, aromatic H); 7.08-7.22 (m, 4H, aromatic H)

HPLC: XBridge tᵣ=13.5 min

ES⁻ mass: [M−H]⁻=255.2

### Example 10

### Summary Table of the Molecules Obtained in Examples 1 to 9

TABLE 1

| Molecule No. | Appearance | ¹H NMR (CDCl₃ or DMSO-d₆) |
|---|---|---|
| Compound 1 | Colourless | 3.14 (t, 2H, CH₂—Ph); 4.24 (t, 2H, CH₂—O); 6.81 (s, 1H, —OH); |
| | oil | 6.85-7.22 (m, 8H, aromatic H) |
| Compound 2 | Colourless | 2.33 (s, 3H, —CH₃); 3.13 (t, 2H, CH₂—Ph); 4.26 (t, 2H, |
| | oil | CH₂—O); 6.81-7.22 (m, 8H, aromatic H) |
| Compound 3 | Yellow oil | 3.14 (t, 2H, CH₂—Ph); 4.24 (t, 2H, CH₂—O); 6.54 (s, 1H, —OH); |
| | | 6.79-6.85 (m, 2H, aromatic H); 6.88-6.94 (m, 2H, H |
| | | aromatic); 7.09-7.26 (m, 2H, aromatic H); 7.36-7.42 (m, |
| | | 2H, aromatic H) |
| Compound 4 | Yellow oil | 3.15 (t, 2H, CH₂—Ph); 4.29 (t, 2H, CH₂—O); 6.70-7.03 (m, |
| | | 5H, aromatic H); 7.15-7.26 (m, 2H, aromatic H); 7.28-7.33 |
| | | (m, 2H, aromatic H) |
| Compound 5 | Colourless | 3.17 (t, 2H, CH₂—Ph); 4.18 (t, 2H, CH₂—O); 6.87-6.94 (m, |
| | oil | 2H, aromatic H); 6.98-7.08 (m, 2H, aromatic H); 7.31-7.41 |
| | | (m, 5H, aromatic H) |
| Compound 6 | Colourless | 3.18 (t, 2H, CH₂—Ph); 4.20 (t, 2H, CH₂—O); 6.82-7.18 (m, |
| | oil | 6H, aromatic H); 7.22-7.37 (m, 2H, aromatic H) |
| Compound 7 | Colourless | 3.26 (t, 2H, CH₂—Ph); 4.20 (t, 2H, CH₂—O); 6.83-6.90 |
| | oil | (m, 2H, aromatic H); 6.95-7.03 (m, 2H, |
| | | aromatic H); 7.12 (m, 1H, aromatic H); |
| | | 7.23-7.37 (m, 2H, aromatic H); 7.60 (m, 1H, aromatic H) |
| Compound 8 | White solid | 3.08 (t, 2H, CH₂—Ph); 4.15 (t, 2H, CH₂—O); 6.81-6.88 (m, |
| | | 2H, aromatic H); 6.95-7.07 (m, 4H, aromatic H); 7.24-7.29 |
| | | (m, 2H, aromatic H) |
| Compound 9 | Beige solid | 3.05 (t, 2H, CH₂—Ph); 3.90 (s, 6H, —OCH₃); 4.13 |
| | | (t, 2H, CH₂—O); 6.76-7.02 (m, 7H, aromatic H) |
| Compound 10 | Colourless | 3.30 (t, 2H, CH₂—Ph); 4.17 (t, 2H, CH₂—O); 6.81-6.88 (m, |
| | oil | 2H, aromatic H); 6.94-7.02 (m, 2H, aromatic H); 7.34-7.37 |
| | | (m, 1H, aromatic H); 7.39-7.55 (m, 2H, aromatic H); 7.67 |
| | | (m, 1H, aromatic H) |
| Compound 11 | Colourless | 2.32 (s, 3H, —CH₃); 3.09 (t, 2H, CH₂—Ph); 4.14 (t, 2H, CH₂—O); |
| | oil | 6.84-6.89 (m, 2H, aromatic H); 6.90-7.10 (m, 2H, H |
| | | aromatic); 7.11-7.14 (m, 3H, aromatic H); 7.23-7.29 (m, |
| | | 1H, aromatic H) |
| Compound 12 | Colourless | 2.41 (s, 3H, —CH₃); 3.17 (t, 2H, CH₂—Ph); 4.17 (t, 2H, CH₂—O); |
| | oil | 6.86-6.93 (m, 2H, aromatic H); 6.99-7.07 (m, 2H, |
| | | aromatic H); 7.21-7.31 (m, 4H, aromatic H) |
| Compound 13 | Colourless | 2.33 (s, 3H, —CH₃); 3.09 (t, 2H, CH₂—Ph); 4.15 (t, 2H, CH₂—O); |
| | oil | 6.83-6.90 (m, 2H, aromatic H); 6.96-7.04 (m, 2H, |
| | | aromatic H); 7.16-7.27 (m, 4H, aromatic H) |
| Compound 14 | Yellow oil | 3.11 (t, 2H, CH₂—Ph); 3.80 (s, 3H, —OCH₃); 3.87 (s, |
| | | 3H, —OCh₃); 4.12 (t, 2H, CH₂—O); 6.82-6.95 (m, 6H, aromatic |
| | | H); 7.23-7.29 (m, 2H, aromatic H) |
| Compound 15 | Golden oil | 3.03 (t, 2H, CH₂—Ph); 3.77 (s, 3H, —OCH₃); 4.08 (t, 2H, |
| | | CH₂—O); 6.77-6.87 (m, 5H, aromatic H); 7.12-7.26 (m, 4H, |
| | | aromatic H) |
| Compound 16 | Colourless | 3.10 (t, 2H, CH₂—Ph); 3.84 (s, 3H, —OCH₃); 4.13 (t, 2H, |
| | oil | CH₂—O); 6.82-7.02 (m, 6H, aromatic H); 7.23-7.28 (m, 2H, |
| | | aromatic H) |
| Compound 17 | Golden oil | 3.12 (t, 2H, CH₂—Ph); 4.22 (t, 2H, CH₂—O); 6.75-6.96 (m, |
| | | 6H, aromatic H); 7.13-7.21 (m, 2H, aromatic H) |
| Compound 18 | Colourless | 1.80 (m, 1H, cyclopropyl); 2.10 (m, 1H, cyclopropyl); 2.70 (m, |
| | oil | 2H, cyclopropyl); 3.86 (s, 3H, —O—CH₃); 4.02 (s, |
| | | 2H, —CH₂—O); 6.62-7.40 (m, 8H, aromatic H) |
| Compound 19 | White solid | 3.02 (t, 2H, CH₂—Ph); 4.27 (t, 2H, CH₂—O); 6.71-7.19 (m, |
| | | 4H, aromatic H); 7.18 (d, 2H, aromatic H); 8.19 (m, 2H, |
| | | aromatic H); 9.54 (s, 1H, —OH) |
| Compound 20 | White solid | 2.94 (t, 2H, CH₂—Ph); 4.05 (t, 2H, CH₂—O); 5.02 (s, 2H, |
| | | benzylic CH₂); 6.71-7.16 (m, 8H, aromatic H); 7.29-7.38 |
| | | (m, 5H, aromatic H); 9.50 (s, 1H, —OH) |
| Compound 21 | Beige solid | 2.90 (t, 2H, CH₂—Ph); 3.96 (t, 2H, CH₂—O); 4.59 (s, 2H, |
| | | NH₂); 6.45-6.51 (d, 2H, aromatic H); 6.62-6.69 (d, 2H, |
| | | aromatic H); 6.72-7.15 (m, 4H, aromatic H); 9.42 (s, 1H, —OH) |
| Compound 22 | Cream- | 1.99 (s, 3H, N—COCH₃); 2.28 (s, 3H, O—COCH₃); 2.91 (t, |
| | coloured | 2H, CH₂—Ph); 3.96 (t, 2H, CH₂—O); 4.08 (s, 2H, NH₂); 6.82- |
| | solid | 6.85 (d, 2H, aromatic H); 7.08-7.43 (m, 4H, aromatic H); |
| | | 7.44-7.47 (d, 2H, aromatic H); 9.78 (s, 1H, —OH) |
| Compound 23 | White solid | 5.28 (s, 2H, CH₂—Ph); 6.82-7.08 (m, 6H, aromatic H); 7.55 |
| | | (dd, 1H, aromatic H); 7.78 (d, 1H, aromatic H); 11.08 (s, |
| | | 1H, —OH) |
| Compound 24 | Yellow oil | 4.06-4.17 (m, 2H, CH₂—Ph); 5.28 (m, 1H, CH—OH); 6.82-7.20 |
| | | (m, 8H, aromatic H); 8.40 (s, 1H, —OH) |
| Compound 25 | White solid | 3.12 (t, 2H, CH₂—Ph); 4.22 (t, 2H, CH₂—O); 5.10 (s, 2H, |
| | | benzylic CH₂); 6.87-7.47 (m, 13H, aromatic H) |
| Compound 26 | Yellow solid | 3.11 (t, 2H, CH₂—Ph); 4.25 (t, 2H, CH₂—O); 5.49 (s, |
| | | 1H, —OH); 6.80-7.25 (m, 8H, aromatic H) |
| Compound 27 | Colourless | 3.13 (t, 2H, CH₂—Ph); 4.14 (t, 2H, CH₂—O); 5.00 (s, |
| | oil | 1H, —OH); 6.75-7.28 (m, 8H, aromatic H) |
| Compound 28 | White solid | 3.14 (t, 2H, CH₂—Ph); 4.16 (t, 2H, CH₂—O); 5.00 (s, 2H, |
| | | benzylic CH₂); 6.83-7.47 (m, 13H, aromatic H) |
| Compound 29 | White solid | 3.04 (m, 2H); 3.12 (m, 1H); 3.28 (t, 2H, CH₂—C₆H₅); 3.46 |
| | | (m, 1H); 3.83 (m, 1H,); 4.16 (t, 2H, CH₂—O); 4.59 (m, 1H, —OH); |
| | | 4.83 (d, 1H); 5.04-5.32 (m, 3H, —OH); 6.93-7.25 (m, |
| | | 8H, aromatic H). |
| Compound 30 | White solid | 2.05 (s, 12H, —CO—CH₃); 3.08 (t, 2H, CH₂—C₆H₅); 3.90 (m, |
| | | 1H, anomeric H); 4.02-4.33 (m, 4H, glucose H); 5.10-5.23 |
| | | (m, 2H, glucose H); 5.35 (t, 2H, CH₂—O); 6.80-7.29 (m, |
| | | 8H, aromatic H). |
| Compound 3 1 | Yellow oil | 3.13 (t, 2H, CH₂—Ph); 4.24 (t, 2H, CH₂—O); 6.85-7.28 (m, |
| | | 8H, aromatic H) |
| Compound 32 | Light yellow | 5.32 (s, 2H, CH₂—Ph); 6.82-7.26 (m, 6H, aromatic H); 7.55 |
| | solid | (dd, 1H, aromatic H); 7.75 (d, 1H, aromatic H) |
| Compound 35 | Colourless | 1.22 (t, 3H, CH₃); 2.60 (q, 2H, CH₂—CH₃); 3.13 (t, 2H, |
| | oil | CH₂—Ph); 4.26 (t, 2H, CH₂—O); 6.84-6.96 (m, 4H, aromatic |
| | | H); 7.10-7.21 (m, 4H, aromatic H) |
| Compound 36 | Beige solid | 1.22 (t, 3H, CH₃); 1.61 (q, 2H, CH₂); 5.31 (s, 2H, CH₂—Ph); |
| | | 6.85-7.16 (m, 6H, aromatic H); 7.55 (m, 1H, aromatic H); |
| | | 7.80 (m, 1H, aromatic H); 11.85 (s, 1H, —OH) |
| Compound 39 | Colourless | 1.22 (s, 6H, CH₃); 2.87 (heptuplet, 1H, CH—); 3.13 (t, 2H, |
| | oil | CH₂—Ph); 4.26 (t, 2H, CH₂—O); 6.85-6.95 (m, 4H, aromatic |
| | | H); 7.13-7.21 (m, 4H, aromatic H) |
| Compound 40 | Yellow solid | 1.23 (s, 6H, CH₃); 2.88 (heptuplet, 1H, CH—(CH₃)₂); 5.30 |
| | | (s, 2H, CH₂—Ph); 6.94 (m, 5H, aromatic H); 7.20 (m, 1H, |
| | | aromatic H); 7.56 (m, 1H, aromatic H); 7.82 (m, 1H, |
| | | aromatic H) |
| Compound 43 | Colourless | 0.66 (t, 3H, —CH₂—CH₃); 1.26 (s, 6H, —C—CH₃); 1.62 (m, |
| | oil | 2H, —C—CH₂—); 3.13 (t, 2H, CH₂—Ph); 4.27 (t, 2H, CH₂—O); 6.85- |
| | | 7.26 (m, 8H, aromatic H) |
| Compound 44 | Beige solid | 0.70 (t, 3H, CH₂—CH₃); 1.30 (s, 6H, CH₃); 1.62 (q, 2H, |
| | | CH₂—CH₃) 5.31 (s, 2H, CH₂—Ph); 6.94 (m, 5H, aromatic H); |
| | | 7.26 (m, 1H, aromatic H); 7.55 (m, 1H, aromatic H); 7.82 |
| | | (m, 1H, aromatic H); 11.87 (s, 1H, —OH) |
| Compound 47 | Colourless | 1.30 (t, 9H, —C—(CH₃)₃); 3.13 (t, 2H, CH₂—Ph); 4.27 (t, 2H, |
| | oil | CH₂—O); 6.86-7.33 (m, 8H, aromatic H) |
| Compound 51 | Colourless | 0.46-1.67 (m, 19H, —(CH₂)₈—CH₃); 3.13 (t, 2H, CH₂—Ph); |
| | oil | 4.27 (t, 2H, CH₂—O); 6.84-6.96 (m, 4H, aromatic H); 7.13- |
| | | 7.24 (m, 4H, aromatic H) |
| Compound 56 | Beige solid | 1.41 (t, 3H, CH₃); 4.00 (q, 2H, CH₂); 5.30 (s, 2H, CH₂—Ph); |
| | | 6.80-7.07 (m, 6H, aromatic H); 7.54 (m, 1H, aromatic H); |
| | | 7.79 (m, 1H, aromatic H); 11.87 (s, 1H, —OH) |
| Compound 57 | White solid | 1.41 (t, 3H, —CH₂—CH₃); 4.07 (dd, 2H, —CH(OH)—CH₂); |
| | | 4.17 (m, 2H, —CH₂—CH₃); 5.27 (d, 1H, —CH(OH)—); 6.83- |
| | | 7.25 (m, 8H, aromatic H) |
| Compound 124 | White solid | 3.91 (s, 3H, —O—CH₃); 5.41 (s, 2H, —CO—CH₂—); 6.86-7.07 |
| | | (m, 6H, aromatic H); 7.52-7.82 (m, 2H, aromatic H) |
| Compound 125 | Brown oil | 3.93 (s, 3H, —O—CH₃); 4.20 (m, 2H, —CH(OH)—CH₂—); 5.23 |
| | | (dd, 1H, —CH(OH)—); 6.84-7.21 (m, 8H, aromatic H) |
| Compound 127 | White solid | 1.22 (d, 3H, —CH₃ Rhamnose); 2.07 (s, 6H, —CO—CH₃); 2.21 |
| | | (s, 3H, —CO—CH₃); 3.15 (t, 2H, CH₂—C₆H₅); 4.00 (m, 1H, |
| | | anomeric H); 4.22 (m, 2H, CH₂—O); 5.15-5.50 (m, 4H, |
| | | Rhamnose H); 6.83-7.30 (m, 8H, aromatic H). |
| Compound 128 | White solid | 1.28 (d, 3H, —CH₃ Rhamnose); 3.07 (m, 2H, CH₂—C₆H₅); |
| | | 4.14 (m, 2H, CH₂—O); 3.51-4.19 (m, 4H, Rhamnose H); |
| | | 5.56 (s, 1H, Rhamnose H); 6.82-7.28 (m, 8H, aromatic H). |
| Compound 129 | Beige solid | 3.79 (s, 3H, —OCH₃); 5.29 (s, 2H, CH₂—Ph); 6.80-7.07 (m, |
| | | 6H, aromatic H); 7.54 (m, 1H, aromatic H); 7.79 (m, 1H, |
| | | aromatic H); 11.86 (s, 1H, —OH) |
| Compound 130 | Colourless | 0.85 (t, 3H, CH₃); 1.49 (q, 2H, CH₂—CH₃); 2.44 (t, |
| | oil | 2H, —CH₂—CH₂); 3.04 (t, 2H, CH₂—Ph); 4.16 (t, 2H, |
| | | CH₂—O); 6.70-7.12 (m, 8H, aromatic H) |
| | | 0.93 (t, 3H, —CH₂—CH₃); 1-60 (m, 2H, —CH₂—CH₃); 2.54 (t, |
| | | 2H, —CH₂—CH₂—); 3.14 (t, 2H, CH₂—Ph); 4.26 (t, 2H, CH₂—O); |
| | | 6.84-6.97 (m, 4H, aromatic H); 7.08-7.22 (m, 4H, |
| | | aromatic H) |
| Compound 133 | Beige solid | 2.62 (m, 3H, —CH₃); 5.31 (s, 2H, —CO—CH₂—); 6.85-7.16 (m, |
| | | 6H, aromatic H); 7.52-7.82 (m, 2H, aromatic H) |
| Compound 134 | Brown oil | 3.79 (s, 3H, —O—CH₃); 4.12 (m, 2H, —CH(OH)—CH₂—); 5.27 |
| | | (dd, 1H, —CH(OH)—); 6.84-7.27 (m, 8H, aromatic H) |
| Compound 135 | Beige solid | 5.04 (s, 2H, —CH₂—Ph); 5.19 (s, 2H, —CO—CH₂—); 6.55 (m, |
| | | 2H, aromatic H); 6.84 (m, 2H, aromatic H); 7.15 (m, 2H, H |
| | | aromatic); 7.40 (m, 5H, aromatic H); 7.56 (m, 1H, H |
| | | aromatic); 8.03 (m, 1H, aromatic H) |
| Compound 136 | Beige solid | 3.83-4.17 (m, 2H, —CH₂—O—); 5.03 (s, 2H, —CH₂—Ph); 5.38 |
| | | (m, 1H, —CH(OH)—); 6.84 (m, 2H, aromatic H); 6.70-7.50 |
| | | (m, 13H, aromatic H) |
| Compound 137 | Beige solid | 3.12 (t, 2H, CH₂—Ph); 3.80 (s, 3H, —OCH₃); 4.24 (t, 2H, |
| | | CH₂—O); 6.80-6.97 (m, 6H, aromatic H); 7.13-7.22 (m, 2H, |
| | | aromatic H) |

### BIOLOGICAL TESTS

### Results for Dermoplasty Maintained in Survival

1. Preparation of the explants
During abdominoplasty of a woman aged 53 years, 45 skin explants were prepared. The explants were put in BEM medium (BIO-EC's Explants Medium) for survival at 37° C. in humid atmosphere, enriched with 5% CO₂.

2. Application of the products
The solutions are prepared in DMSO from pure products, in order to obtain final concentrations of 2, 20 and 100 μg by incorporating an identical volume of 10 μl in each well and for each treatment. The treatment was carried out in the survival medium on days 0, 1, 2, 5 and 7.

3. Taking samples
On D0, the 3 explants of batch TO were taken and each explant was cut in two. One part was fixed in buffered formol for observation of the general morphology. The other was frozen and stored at −80° C.
On D5 and D9, 3 explants from each batch were taken and treated in the same way.

4. Histological treatment
After fixation for 48 hours in buffered formol, the samples were dehydrated and embedded in paraffin using a Leica 1020 automatic dehydrator. They were embedded according to procedure MO-H-153 using a Leica EG 1160 coating station. Sections of 5 μm were prepared according to procedure MO-H-173 using a Minot-type microtome, Leica RM 2125 and mounted on Superfrost® histological glass slides.

5. Microscopic observations
The microscopic observations were carried out by light microscopy and fluorescence microscopy, using a Orthoplan-type Leica microscope, with ×25 objective. The images were recorded with a Sony DXC 390P tri CCD camera and stored using the Leica IM1000 data archiving software.
The general morphology was observed on sections in paraffin after staining with Masson trichrome, Goldner variant according to procedure MO-H-157.

6. Staining and immunolabelling

Immunolabelling will be carried out on sections with the appropriate antibody. The nuclei may be stained with propidium iodide.

### Example 11

### Histological Assessment of the General Morphology

Compound 1, of formula

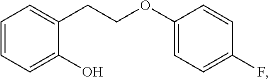 [SMILES: Oc1ccccc1CCOc1ccc(F)cc1]

and the reference compound, (+)-dehydroisoandrosterone (DHEA Acros ref. 154980100), were tested at a concentration of 2 μg/mL in DMSO, on abdominoplasties. After 9 days of survival, compound 1 displayed a very clear epidermal and dermal restructuring activity: the epidermal structure is clearly acanthotic (thicker) with a good morphology, and the papillary dermis is clearly denser. In contrast, the reference product, (+)-dehydroisoandrosterone, does not alter the epidermal structure or the papillary dermis at a concentration of 2 μg/mL.

### Example 12

### Staining and Immunolabelling

A more detailed study using staining and immunolabelling of the sections from the aforementioned plasty procedures treated with compound 1 shows
clear overexpression of collagen I in the papillary dermis
very slight densification of collagen III in the papillary dermis
clear overexpression of collagen IV along the dermal-epidermal junction
moderate overexpression of collagen VII along the dermal-epidermal junction.

Effects of Compound 1 on Expression of Markers by Keratinocytes

The expression of the specific markers was evaluated by RT-qPCR (reverse transcription quantitative polymerase chain reaction) on messenger RNAs extracted from the cell lawns from each treatment. The transcriptomic profile contains 64 genes selected for their importance in keratinocyte differentiation. The PCR reactions (polymerase chain reactions) were carried out by quantitative PCR with the “Light Cycler” system (Roche Molecular System Inc.) according to the procedures recommended by the supplier.

### Example 13

### Analysis of Differential Expression-PCR Arrays

Normal human epidermal keratinocytes were incubated in the presence of compound 1 or a reference with calcium chloride. The expression of the specific markers was evaluated by RT-qPCR on the messenger RNAs extracted from the cell lawns from each treatment.

Under the experimental conditions of this study, compound 1 tested at 10 μM showed an effect that was less, but similar to calcium chloride at 1.5 mM in stimulating the expression of markers involved in lipid synthesis, antimicrobial defence, innate immunity and cell-cell interactions. Moreover, in parallel with this effect, inhibition of expression of the “matrix metallopeptidase” gene MMP9 involved in the degradation of the extracellular matrix and of the stress protein HSPB1, “heat shock protein beta-1”, was also observed.

Compound 1 therefore displayed a pro-differentiating effect.

### Example 14

### Investigation of Effects of Compound 1 on Epidermal Differentiation

Transglutaminase K, TGK, and filaggrin are two protein markers of epidermal differentiation involved in arrangement of the keratin filaments and formation of the envelope of the stratum corneum. In situ immunolabelling of TGK and filaggrin in normal human epidermal keratinocytes cultured in the presence of calcium chloride or of compound 1 showed concentration-dependent stimulation of protein expression of filaggrin and TGK by compound 1.

### Example 15

### Investigation of Anti-Inflammatory Effects of Compound 1

The effects of compound 1 were evaluated on two routes of inflammation, the “chemokine” route and the “prostaglandin” route, by measuring the release of interleukin-8 (IL-8) and of prostaglandin E₂ (PGE₂) by a human keratinocyte line NCTC-2544 stimulated by phorbol-myristate acetate (PMA).

Compound 1 displays a clear anti-inflammatory effect by concentration-dependent inhibition of the release of PGE₂ without altering the release of IL-8.

### Example 16

### Investigation of Effects of Compound 1 on Dermal Reinforcement

The effects of this compound were evaluated on a monolayer of normal human dermal fibroblasts by measuring the neosynthesis of glycosaminoglycans designated as sulphated GAGs (incorporation of [³⁵S]-sulphate). Compound 1 stimulated the neosynthesis of sulphated GAGs, in a non-concentration-dependent fashion. This might explain the effect of dermal reinforcement already observed on skin explants ex vivo.

Evaluation of the Depigmenting Effect on the B16 Melanocyte Line Stimulated by NDP-MSH

### Example 17

### Evaluation of Anti-Melanogenesis Activity

The anti-melanogenesis activity of the compounds in solution in DMSO was evaluated by measuring melanin synthesis in a model of B16 melanocytes stimulated with a stable derivative of α-MSH (natural melanogenesis stimulating hormone): NDP-MSH.

### Culture and Treatments

The melanocytes were seeded in a 96-well plate and were cultured for 24 h (37° C., 5% CO₂, DMEM 1 g/L glucose without phenol red supplemented with glucose at 3 g/L, L-glutamine at 2 mM, penicillin at 50 U/mL, streptomycin at 50 μg/mL, foetal calf serum (FCS) at 10%). After incubation, the culture medium was then replaced with culture medium supplemented or not supplemented (unstimulated control) with a stable derivative of α-MSH and containing or not containing (controls) the test compounds or the reference (kojic acid at 25, 100, 400, 800 μg/mL). Each experiment was carried out with n=3, apart from the controls carried out with n=6, then the cells were incubated for 72 h. Wells without cells received in parallel the same quantities of medium supplemented or not supplemented with NDP-MSH and containing or not containing the test compounds or the reference in order to quantify the background noise associated with the presence of the compounds.

### Determination of Melanin

After incubation for 72 hours, the total melanin (intra- and extracellular) was quantified by measuring the absorption at 405 nm of each sample (direct reading of the culture plates) against a standard range of melanin (melanin concentrations tested from 0.78 to 100 μg/mL). The background noise, measured in the wells without cells, was subtracted from the measured values so as only to take into account the effect connected with melanin production, without taking into account the possible interference connected with the presence of the compounds. The results were expressed as percentage of melanin relative to the control as well as percentage inhibition.

### Evaluation of Cell Viability—MTT Reduction Test

At the end of the treatment, the cells were incubated in the presence of MTT (tetrazolium salt), the transformation of which to blue crystals of formazan is proportional to the activity of succinate dehydrogenase (mitochondrial enzyme). After dissociation of the cells, the formazan was dissolved in DMSO medium and the optical density (OD), representative of the number of living cells and of their metabolic reactivity, was measured with a microplate reader at 540 nm (VERSAmax, Molecular Devices).

The results obtained are presented in the following table:

TABLE 2

| | | | Viability (MTT) |
|---|---|---|---|
| Name and concentrations tested | | Standardized data | % stimulated |
| (μM) | | Inhibition (%) | control |
| Stimulated | 10⁻⁷ M | 0 | 100 |
| control | | | |
| Unstimulated | — | 100 | 100 |
| control | | | |
| Kojic acid | 25 μg/mL | 24 | 85 |
| | 100 μg/mL | 78 | 76 |
| | 400 μg/mL | 90 | 76 |
| Compound 1 | 10 | 0 | 83 |
| | 30 | 93 | 86 |
| | 100 | 112 | 22 |
| Compound 2 | 30 | 100 | 108 |
| | 100 | 112 | 34 |
| Compound 3 | 30 | 110 | 96 |
| | 100 | 112 | 0 |
| Compound 4 | 30 | 95 | 97 |
| | 100 | 111 | 54 |
| Compound 5 | 30 | 49 | 103 |
| | 100 | 111 | 67 |
| Compound 8 | 30 | −27 | 94 |
| | 100 | 50 | 93 |
| Compound 10 | 30 | −11 | 87 |
| | 100 | 27 | 91 |
| Compound 11 | 30 | −2 | 83 |
| | 100 | 44 | 109 |
| Compound 14 | 30 | −22 | 89 |
| | 100 | 100 | 80 |
| Compound 16 | 30 | −33 | 87 |
| | 100 | 52 | 83 |
| Compound 18 | 30 | −21 | 80 |
| | 100 | 105 | 75 |
| Compound 19 | 30 | 82 | 91 |
| | 100 | 111 | 0 |
| Compound 20 | 3 | 83 | 106 |
| | 10 | 108 | 84 |
| | 30 | 115 | 59 |
| Compound 22 | 30 | −3 | 97 |
| | 100 | 61 | 86 |
| Compound 24 | 30 | −12 | 76 |
| | 100 | 84 | 78 |
| Compound 25 | 30 | 46 | 93 |
| Compound 27 | 30 | 95 | 77 |
| Compound 30 | 30 | 13 | 88 |
| Compound 31 | 3 | 20 | 91 |
| | 10 | 70 | 104 |
| | 30 | 109 | 88 |
| Compound 35 | 3 | 54 | 105 |
| | 10 | 92 | 97 |
| | 30 | 112 | 88 |
| Compound 39 | 3 | 70 | 119 |
| | 10 | 111 | 101 |
| | 30 | 119 | 86 |
| Compound 40 | 10 | 5 | 102 |
| | 30 | 58 | 90 |
| Compound 43 | 3 | 88 | 116 |
| | 10 | 119 | 90 |
| | 30 | 120 | 9 |
| Compound 44 | 10 | 24 | 89 |
| | 30 | 117 | 80 |
| Compound 47 | 3 | 75 | 116 |
| | 10 | 111 | 89 |
| | 30 | 116 | 18 |
| Compound 51 | 3 | 104 | 107 |
| | 10 | 116 | 47 |
| | 30 | 115 | 0 |
| Compound 57 | 30 | 47 | 97 |
| Compound 125 | 30 | 13 | 90 |
| Compound 127 | 30 | 29 | 90 |
| Compound 130 | 3 | 72 | 98 |
| | 10 | 110 | 89 |
| | 30 | 116 | 81 |

### Example 18

### Evaluation of the Depigmenting Effect Compared with 2 Reference Molecules

The operating conditions are the same as in Example 12.

The effects of the following three molecules were compared:
compound 1
4-(2,4-dihydroxyphenyl)butane [18979-61-8], or rucinol, ref. 1
1-(2,4-dihydroxyphenyl)-3-(2,4-dimethoxy-3-methylphenyl)propane [869743-37-3], Unigen Pharmaceuticals patent US2005/267047, ref. 2

The following table summarizes the results obtained:

TABLE 3

| | | | Viability (MTT) |
|---|---|---|---|
| | | Standardized data | % stimulated |
| Name and concentrations tested (μM) | | Inhibition (%) | control |
| Stimulated control | 10⁻⁷M | 0 | 100 |
| Unstimulated control | — | 100 | 100 |
| Compound 1 | 30 | 98 | 85 |
| | 100 | 124 | 13 |
| 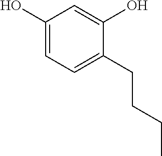 Ref 1 [18979-61-8] | 30 100 | 82 100 | 107 107 |
| 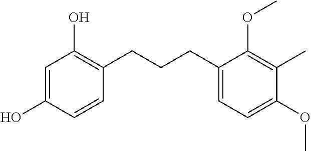 [SMILES: COc1ccc(CCCc2ccc(O)cc2O)c(OC)c1C] Ref 2 [869743-37-3] | 30 100 | 45 113 | 102 76 |

Compound 1 therefore displays a clear inhibitory effect at the two concentrations tested and is slightly more effective than the two reference products used.

### Example 19

### Activity of Compound 1 on the PPAR Receptors (Peroxisome Proliferator Activated Receptor)

### Affinity Assays

The affinity of compound 1 for the PPAR receptors (α and γ) was tested by in vitro binding assays (tests carried out by the company Cerep).

This is a nuclear receptor competitive binding assay, between a radiolabelled ligand and the test molecule (here compound 1).

The principle of the technique consists of measuring the displacement of the radioactive ligand bound to the receptor by the test molecule.
Material and Methods

The specific binding of the ligand is defined by the difference between total binding and non-specific binding determined in the presence of an excess of unlabelled ligand.

The results are expressed as:
percentage of control specific binding ((measured specific binding/control specific binding)×100))
percentage of inhibition of control specific binding ((100−(measured specific binding/control specific binding))
   obtained in the presence of compound 1.

The values of IC₅₀ (concentration at which the maximum control specific binding is inhibited by 50%), and the Hill coefficient (nH) are determined by non-linear regression analysis of the curves of competition with a mean replicated value using adjustment of Hill's equation

(Y=D+[(A−D)/(1+(C/C₅₀)^{nH})]).

in which,
Y=specific binding
D=minimum specific binding
A=maximum specific binding
C=concentration of the compound
C₅₀═IC₅₀
nH=slope of the straight line

This analysis was carried out using software developed by the company Cerep (Hill Software) and was validated by comparison with the data generated by the software SigmaPlot® 4.0 for Windows (® 1997 by SPSS Inc).

The inhibition constant (Ki) was calculated using the Cheng-Prusoff equation

[Ki=IC₅₀/(1+(L/K_{D})],

in which
L=concentration of the radioligand in the assay
K_{D}=affinity of the radioligand for the receptor. K_{D} was determined by a Scatchard analysis:

TABLE 4

| | | | | | | | Method of |
|---|---|---|---|---|---|---|---|
| Assay | Source | Ligand | Conc. | K_{D} | Non-specific | Incubation | detection |
| PPAR | Human | [³H]WY | 200 nM | 4000 | GW 7647 | 120 min | Scintillation |
| α | recombinant | 14143 | | nM | (10 μM) | 4° C. | detector |
| | (E. coli) | | | | | | |
| PPAR | Human | [³H] | 5 nM | 5.7 | Rosiglitazone | 120 min | Scintillation |
| γ | recombinant | rosiglitazone | | nM | (10 μM) | 4° C. | detector |
| | (E. coli) | | | | | | |

Results:

TABLE 5

| | | | |
|---|---|---|---|
| | | | % Inhibition of |
| | | | control specific |
| PPAR receptor | Test compound | Concentration (M) | binding |

PPARα (h) (agonist radioligand)

| | | | |
|---|---|---|---|
| 19343-1 | Compound 1 | 10⁻⁷ | −5 |
| | | 10⁻⁶ | −2 |
| | | 10⁻⁵ | 0 |

PPARγ (h) (agonist radioligand)

| | | | |
|---|---|---|---|
| 19343-1 | Compound 1 | 10⁻⁷ | 0 |
| | | 10⁻⁶ | 1 |
| | | 10⁻⁵ | 49 |

At different concentrations of compounds, and with either the receptor PPARα or PPARγ, the percentage of inhibition is close to zero. Therefore there is no activity of compound 1 with respect to the PPAR receptors.

The 49% value indicates a slight to moderate effect.

### Assays of Activity

The agonist or antagonist activity of compound 1 on the PPAR receptors was tested by fluorescence techniques.
Material and Methods

The results are expressed as:
percentage of control specific agonist response (measured specific response/control specific agonist response)×100) and
percentage inhibition of the control specific agonist response (100−((measured specific response)/control specific agonist response)×100)) obtained in the presence of compound 1.

The EC₅₀ values (concentration at which the maximum specific response is 50%) and the IC₅₀ values (concentration at which the maximum control specific agonist response is inhibited by 50%) are determined by non-linear regression analysis of the concentration-response curves with a mean replicated value using adjustment of Hill's equation

(Y=D+[(A−D)/(1+(C/C₅₀)^{nH})]

in which,
Y=specific response
D=minimum specific response
A=maximum specific response
C=concentration of the compound
C₅₀=IC₅₀ or EC₅₀
nH=slope of the straight line

This analysis was carried out using software developed by the company Cerep (Hill Software) and validated by comparison with the data generated by the software SigmaPlot® 4.0 for Windows (® 1997 by SPSS Inc).

For the antagonists, the apparent dissociation constants (K_{B}) were calculated using the modified Cheng-Prusoff equation

K_{B}=IC₅₀/(1+(A/EC₅₀A)),

in which
A=concentration of the reference agonist in the assay
EC₅₀A=EC₅₀ value of the reference agonist.

TABLE 6

| | | | | Component | |
|---|---|---|---|---|---|
| Assay | Source | Stimulus | Incubation | measured | Method of detection |
| PPARα (h) | Human | /(1 μM GW 7647 | RT | Coactivator | Alpha screen |
| (agonist | recombinant | for the control) | | | |
| effect) | | | | | |
| PPARα (h) | Human | GW 7647 | RT | Coactivator | Alpha screen |
| (antagonist | recombinant | (100 nM) | | | |
| effect) | | | | | |
| PPARδ (h) | Human | /(100 nM of GW | RT | Coactivator | Alpha screen |
| (agonist | recombinant | 0742 for the | | | |
| effect) | | control) | | | |
| PPARδ (h) | Human | GW 0742 | RT | Coactivator | Alpha screen |
| (antagonist | recombinant | (10 nM) | | | |
| effect) | | | | | |
| PPARγ (h) | Human | /(10 μM of | RT | Coactivator | Alpha screen |
| (agonist | recombinant | rosiglitazone for | | | |
| effect) | | the control) | | | |
| PPARγ (h) | Human | Rosiglitazone | RT | Coactivator | Alpha screen |
| (antagonist | recombinant | (1 μM) | | | |
| effect) | | | | | |

Results for agonist activity

TABLE 7

| | | | |
|---|---|---|---|
| | | | % Control agonist |
| PPAR receptor | Test compound | Concentration (M) | response |

PPARα (h) (agonist effect)

| | | | |
|---|---|---|---|
| 19343-1 | Compound 1 | 10⁻⁷ | −1 |
| | | 10⁻⁶ | 0 |
| | | 10⁻⁵ | 2 |

PPARδ (h) (agonist effect)

| | | | |
|---|---|---|---|
| 19343-1 | Compound 1 | 10⁻⁷ | 2 |
| | | 10⁻⁶ | 4 |
| | | 10⁻⁵ | 2 |

PPARγ (h) (agonist effect)

| | | | |
|---|---|---|---|
| 19343-1 | Compound 1 | 10⁻⁷ | 0 |
| | | 10⁻⁶ | 0 |
| | | 10⁻⁵ | 0 |

As the percentage of response is low (−1 to 4), compound 1 is therefore not an agonist.
Results for antagonist activity

TABLE 8

| | | | |
|---|---|---|---|
| | | | % Inhibition of |
| | | | control agonist |
| PPAR receptor | Test compound | Concentration (M) | response |

PPARα (h) (antagonist effect)

| | | | |
|---|---|---|---|
| 19343-1 | Compound 1 | 10⁻⁷ | 1 |
| | | 10⁻⁶ | 0 |
| | | 10⁻⁵ | 4 |

PPARδ (h) (antagonist effect)

| | | | |
|---|---|---|---|
| 19343-1 | Compound 1 | 10⁻⁷ | 0 |
| | | 10⁻⁶ | −2 |
| | | 10⁻⁵ | −5 |

PPARγ (h) (antagonist effect)

| | | | |
|---|---|---|---|
| 19343-1 | Compound 1 | 10⁻⁷ | 3 |
| | | 10⁻⁶ | 0 |
| | | 10⁻⁵ | 0 |

As the percentage inhibition is low (−5 to 4), compound 1 is therefore not an antagonist.

These various tests therefore prove that compound 1 is not a PPAR activator.

1. A method for preparing cosmetic compositions having anti-ageing and/or depigmenting properties, comprising adding to said compositions compounds of general formula I 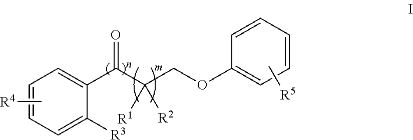
in which
n and m are equal to 0 or 1, their sum always being equal to 1,
R¹ and R², which may be identical or different, represent
hydrogen atoms,
halogen atoms selected from fluorine, chlorine, bromine or iodine,
linear or branched alkyl groups comprising from 1 to 10 carbon atoms, in particular the methyl, ethyl, isopropyl groups, optionally substituted with one or more halogens selected from fluorine, chlorine, bromine or iodine, with a —CF₃ group, with a hydroxy,
—OH groups,
linear or branched alkoxy groups comprising from 1 to 10 carbon atoms, in particular methoxy, ethoxy, optionally substituted with one or more halogens selected from fluorine, chlorine, bromine or iodine, and in particular the trifluoromethoxy group —OCF₃,
phenoxy groups —OPh,
aryloxy groups —OAr in which Ar represents an aromatic group comprising from 6 to 12 carbon atoms optionally substituted with one or more halogen atoms selected from fluorine, chlorine, bromine, or optionally substituted with one or more —OH groups in free or protected form, and in particular protected in the form of —OMes group, —OTHP group of formula 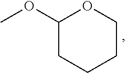
group derived from ethylene glycol of formula 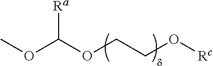
in which
δ varies from 1 to 12,
R^{a} represents a hydrogen or a linear or branched alkyl group comprising from 1 to 6 carbon atoms,
R^{c} represents a linear or branched alkyl group with 1 to 10 carbon atoms,
group derived from propylene glycol of formula, 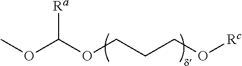
in which
δ′ varies from 1 to 5,
R^{a} and R^{c} have the meanings stated above, group derived from a glycoside compound that may be α- or β-furanose or α- or β-pyranose,
siloxy group of formula —OSi(R^{a})₃ in which R^{a} has the meanings stated above,
—OSitBdPh group of formula 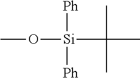 [SMILES: CO[Si](c1ccccc1)(c1ccccc1)C(C)(C)C]
or —OSitBdM group of formula, 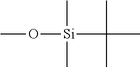
benzyloxy groups —OCH₂Ph,
alkenyl groups comprising from 2 to 10 carbon atoms, linear or branched and comprising at least one C═C double bond,
acyloxy groups comprising from 2 to 10 carbon atoms, derived from carboxylic acids,
—OTHP groups of the above formula,
—OMes groups,
groups derived from ethylene glycol of formula 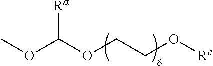
in which
δ varies from 1 to 12,
R^{a} represents a hydrogen or a linear or branched alkyl group comprising from 1 to 6 carbon atoms,
R^{c} represents a linear or branched alkyl group with 1 to 10 carbon atoms,
groups derived from propylene glycol of formula, 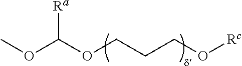
in which
δ′ varies from 1 to 5,
R^{a} and R^{c} have the meanings stated above,
—OH groups optionally coupled to glycoside compounds that may be α- or β-furanose or α- or β-pyranose,
siloxy groups of formula —OSi(R^{a})₃ in which R^{a} has the meanings stated above,
—OSitBdPh groups of formula 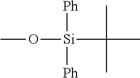 [SMILES: CO[Si](c1ccccc1)(c1ccccc1)C(C)(C)C]
—OSitBdM groups of formula, 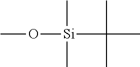
said R¹ and R² optionally forming a ring comprising 3 carbon atoms, said ring having the following formula 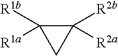
in which
R^{1a}, R^{1b}, R^{2a} and R^{2b}, which may be identical or different, represent
hydrogen atoms,
halogen atoms selected from fluorine, chlorine, bromine or iodine,
linear or branched alkyl groups comprising from 1 to 10 carbon atoms, in particular the methyl, ethyl, isopropyl groups, optionally substituted with one or more halogens selected from fluorine, chlorine, bromine or iodine, with a —CF₃ group, with a hydroxy,
provided that R³ is in this case a hydroxy group —OH,
R³ and R⁴, which may be identical or different, represent
hydrogen atoms,
halogen atoms selected from fluorine, chlorine, bromine,
linear, branched or cyclic alkyl groups comprising from 1 to 10 carbon atoms, in particular the methyl, ethyl, isopropyl groups, optionally substituted with one or more halogens selected from fluorine, chlorine, bromine or iodine, with a —CF₃ group, with a hydroxy,
hydroxyl groups —OH,
linear or branched alkoxy groups comprising from 1 to 10 carbon atoms, in particular the methoxy, ethoxy, isopropoxy, tert-butoxy groups, optionally substituted with one or more halogens selected from fluorine, chlorine, bromine or iodine, and in particular the trifluoromethoxy group —OCF₃,
benzyloxy groups,
an alkenyloxy group comprising from 2 to 10 carbon atoms, linear or branched and comprising at least one C═C double bond,
an alkynyloxy group comprising from 2 to 10 carbon atoms, linear or branched and comprising at least one C≡C triple bond,
—OTHP groups of formula 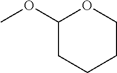
substituted —OTHP groups of the following formula 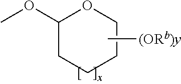
in which
x is equal to 0 or 1, y varies from 0 to 4,
R^{b} is a hydrogen or an acetyl —C(O)—CH₃,
groups derived from ethylene glycol of formula 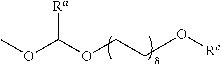
in which
δ varies from 1 to 12,
R^{a} represents a linear or branched alkyl group comprising from 1 to 6 carbon atoms,
R^{c} represents a linear or branched alkyl group with 1 to 10 carbon atoms,
groups derived from propylene glycol of formula 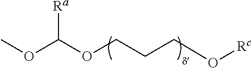
in which
δ′ varies from 1 to 5,
R^{a} and R^{c} have the meanings stated above,
—OH groups optionally coupled to a glycoside compound, which may be an α- or β-furanose or an α- or β-pyranose,
a siloxy group of formula —OSi(R^{a})₃ in which R^{a} has the meanings stated above,
—OSitBdPh of formula 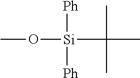 [SMILES: CO[Si](c1ccccc1)(c1ccccc1)C(C)(C)C]
—OSitBdM of formula, 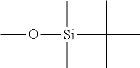
R⁵ represents
a hydrogen atom,
a halogen atom selected from fluorine, chlorine, bromine,
a linear, branched or cyclic alkyl group comprising from 1 to 10 carbon atoms, in particular a methyl, ethyl, isopropyl group, optionally substituted with one or more halogens selected from fluorine, chlorine, bromine or iodine, with a —CF₃ group, with a hydroxy,
an alkenyl group comprising from 2 to 6 carbon atoms, linear or branched and comprising at least one C═C double bond,
an alkynyl group comprising from 2 to 6 carbon atoms, linear or branched and comprising at least one C≡C triple bond,
a phenyl group optionally substituted with a halogen atom, a —CF₃ group, a linear or branched alkyl group comprising from 1 to 10 carbon atoms, a linear or branched alkoxy group comprising from 1 to 10 carbon atoms,
a hydroxyl group —OH,
a linear or branched alkoxy group comprising from 1 to 10 carbon atoms, in particular the methoxy, ethoxy, isopropoxy, tert-butoxy groups, optionally substituted with one or more halogens selected from fluorine, chlorine, bromine or iodine, and in particular the trifluoromethoxy group —OCF₃,
a benzyloxy group,
a phenoxy group,
a thiophenyl group,
an acyloxy group comprising from 2 to 10 carbon atoms, linear or branched,
an —OTHP group of formula 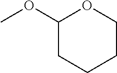
a substituted —OTHP group of the following formula 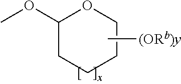
in which
x is equal to 0 or 1, y varies from 0 to 4,
R^{b} is a hydrogen or an acetyl —C(O)—CH₃,
an —OSO₂R^{o} group in which R^{c} is an alkyl group comprising from 1 to 6 carbon atoms, in particular methyl or ethyl, or an aromatic group comprising from 6 to 18 carbon atoms, optionally substituted with one or more halogen atoms, hydroxy or alkoxy group, nitro,
an —OSO₃M group in which M represents an Na⁺ or K⁺ ion,
a group derived from ethylene glycol of formula 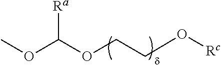
in which
δ varies from 1 to 12,
R^{a} represents a linear or branched alkyl group comprising from 1 to 6 carbon atoms,
R^{c} represents a linear or branched alkyl group with 1 to 10 carbon atoms,
a group derived from propylene glycol of formula 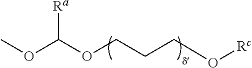
in which
δ′ varies from 1 to 5,
R^{a} and R^{c} have the meanings stated above,
an —OH group optionally coupled to a glycoside compound, which may be an α- or β-furanose or an α- or β-pyranose,
a siloxy group of formula —OSi(R^{a})₃ in which R^{a} has the meanings stated above,
—OSitBdPh of formula 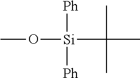 [SMILES: CO[Si](c1ccccc1)(c1ccccc1)C(C)(C)C]
—OSitBdM of formula, 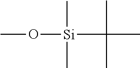
—COOH,
—CN,
—NH₂,
—NH₃⁺, X⁻
—NR^{d}R^{e},
—NHR^{d}R^{e+}, X⁻
—NHCOR^{f},
—NHCOOR^{g},
—NO₂,
R^{d}, R^{e} representing linear or branched alkyl groups comprising from 1 to 4 carbon atoms, optionally substituted with one or more halogen atoms, or carbon chains interrupted by oxygen or sulphur atoms, benzyl groups optionally substituted with a halogen atom, a hydroxy group, an alkoxy group comprising from 1 to 8 carbon atoms,
R^{f} represents a linear or branched alkyl group containing from 1 to 4 carbon atoms, a phthalimido group (in this case NH is replaced with N), a benzyl group optionally substituted with a halogen atom, a hydroxy group, an alkoxy group and in particular substituted with the methoxy group in the para position,
R^{g} represents a linear or branched alkyl group comprising from 1 to 4 carbon atoms, optionally substituted with one or more halogen atoms, or a carbon chain interrupted by oxygen or sulphur atoms, a phenyl group, a benzyl group optionally substituted with a halogen atom, a hydroxy group, an alkoxy group and in particular substituted with the methoxy group in the para position,
X⁻ representing a halide ion,
a group derived from piperazine, in particular 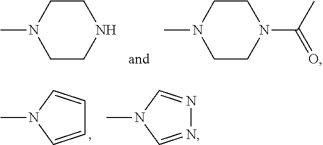
provided that R³ and R⁴ do not represent simultaneously —OH groups or groups comprising an oxygen atom bound to the phenyl, said compounds of Formula I being used in racemic form or as a single enantiomer.

2. The method according to claim 1, of the compounds of Formula II 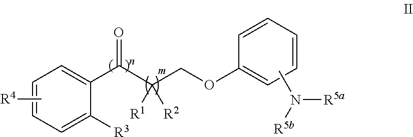
in which
n is equal to 0 or 1, m is equal to 0 or 1, provided that n+m=1,
R¹, R², R³ and R⁴ have the meanings designated above,
—NR^{5a}R^{5b} represents a group comprising a nitrogen atom fixed to the ring, optionally in the form of its salt, this group being selected from
—NH₂,
—NR^{d}R^{e},
—NHCOR^{f},
—NHCOOR^{g},
—NO₂,
a group derived from piperazine, in particular 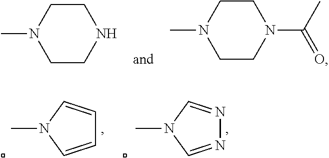
R^{d}, R^{e} representing linear or branched alkyl groups comprising from 1 to 4 carbon atoms, optionally substituted with one or more halogen atoms, or carbon chains interrupted by oxygen or sulphur atoms, benzyl groups optionally substituted with a halogen atom, a hydroxy group, an alkoxy group comprising from 1 to 8 carbon atoms,
R^{f} represents a linear or branched alkyl group containing from 1 to 4 carbon atoms, a phthalimido group (in this case NH is replaced with N), a benzyl group optionally substituted with a halogen atom, a hydroxy group, an alkoxy group and in particular substituted with the methoxy group in the para position,
R^{g} represents a linear or branched alkyl group comprising from 1 to 4 carbon atoms, optionally substituted with one or more halogen atoms, or a carbon chain interrupted by oxygen or sulphur atoms, a phenyl group, a benzyl group
optionally substituted with a halogen atom, a hydroxy group, an alkoxy group and in particular substituted with the methoxy group in the para position.

3. The method according to claim 1, of the compounds of Formula III 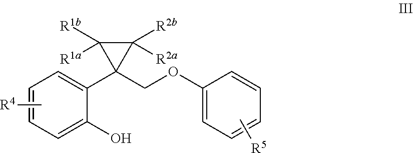
in which
R⁴ and R⁵ have the meanings designated above,
R^{1a}, R^{1b}, R^{2a} and R^{2b}, which may be identical or different, represent
hydrogen atoms,
halogen atoms selected from fluorine, chlorine, bromine or iodine,
linear or branched alkyl groups comprising from 1 to 10 carbon atoms, in particular the methyl, ethyl, isopropyl groups, optionally substituted with one or more halogens selected from fluorine, chlorine, bromine or iodine, with a —CF₃ group, with a hydroxy.

4. The method according to claim 1, of the compounds of Formula 1V 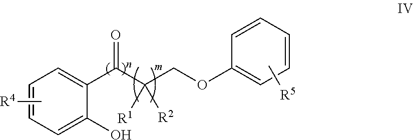
in which
n is equal to 0 or 1, m is equal to 0 or 1, provided that n+m=1,
R¹, R², R⁴ and R⁵ have the meanings designated above,
or of the compounds of Formula 1VA 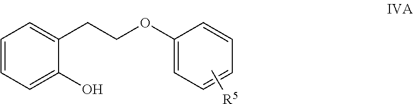
in which R⁵ has the meaning designated above,
or of the compounds of Formula 1 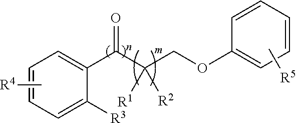
in which
n is equal to 0 or 1, m is equal to 0 or 1, provided that n+m=1,
R¹, R², R³, R⁴ and R⁵ have the meanings designated above,
provided that R³ and R⁴ are both different from —OH or are of any form in which —OH is protected,
or of the compounds of Formula V 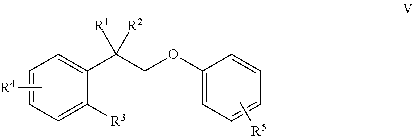
in which
R¹, R², R³, R⁴ and R⁵ have the meanings designated above,
provided that R³ and R⁴ are both different from —OH or are of any form in which —OH is protected,
or of the compounds of Formula VI 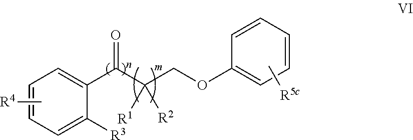
in which
n is equal to 0 or 1, m is equal to 0 or 1, provided that n+m=1,
R¹, R², R³ and R⁴ have the meanings designated above,
R^{5c} is a halogen atom selected from fluorine, chlorine or bromine,
or of the compounds of Formula VII 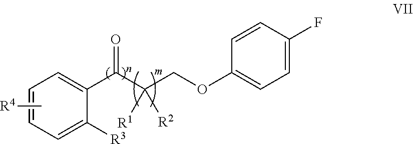
in which
n is equal to 0 or 1, m is equal to 0 or 1, provided that n+m=1,
R¹, R², R³ and R⁴ have the meanings designated above,
or of the compounds of Formula VIII 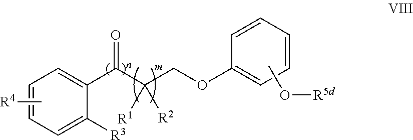
in which
n is equal to 0 or 1, m is equal to 0 or 1, provided that n+m=1,
R¹, R², R³ and R⁴ have the meanings designated above,
—O—R^{5d} is a group selected from
—OH,
linear or branched alkoxy groups comprising from 1 to 10 carbon atoms, in particular the methoxy, ethoxy, isopropoxy, tert-butoxy groups, optionally substituted with one or more halogens selected from fluorine, chlorine, bromine or iodine, and in particular the trifluoromethoxy group —OCF₃,
benzyloxy groups,
phenoxy groups,
—OTHP groups of formula 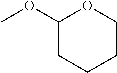
substituted —OTHP groups of the following formula 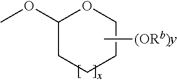
in which
x is equal to 0 or 1, y varies from 0 to 4,
R^{b} is a hydrogen or an acetyl —C(O)—CH₃,
—OSO₂R^{c} groups in which R^{c} is an alkyl group comprising from 1 to 6 carbon atoms, in particular methyl or ethyl, or an aromatic group comprising from 6 to 18 carbon atoms, optionally substituted with one or more halogen atoms, hydroxy or alkoxy group, nitro,
—OSO₃M groups in which M represents an Na⁺ or K⁺ ion,
groups derived from ethylene glycol of formula 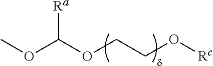
in which
δ varies from 1 to 12,
R^{a} represents a linear or branched alkyl group comprising from 1 to 6 carbon atoms,
R^{c} represents a linear or branched alkyl group with 1 to 10 carbon atoms,
groups derived from propylene glycol of formula 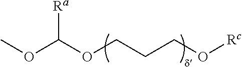
in which
δ′ varies from 1 to 5,
R^{a} and R^{c} have the meanings stated above,
—OH groups optionally coupled to a glycoside compound, which may be an α- or β-furanose or an α- or β-pyranose,
or of the compounds of Formula 1X 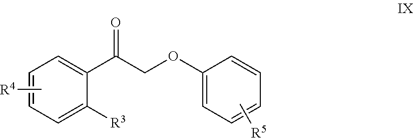
in which R³, R⁴ and R⁵ have the meanings designated above,
or of the compounds of Formula X 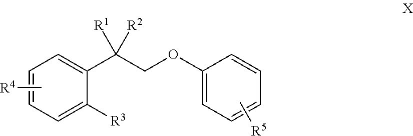
in which R¹, R², R³, R⁴ and R⁵ have the meanings designated above,
or of the compounds of Formula XA 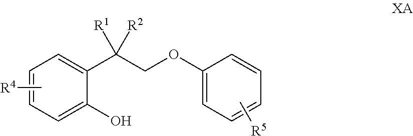
in which
R¹, R², R⁴ and R⁵ have the meanings designated above,
or of the compounds of Formula XI 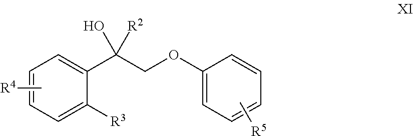
in which R², R³, R⁴ and R⁵ have the meanings designated above,
or of the compounds of Formula XIIA, XIIB and XIIC 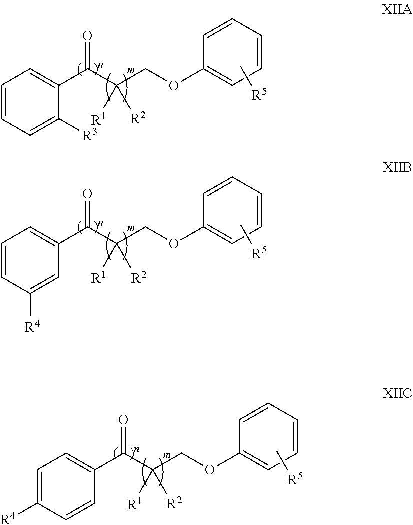
in which
R¹, R², R³, R⁴ and R⁵ have the meanings designated above,
n is equal to 0 or 1, m is equal to 0 or 1, provided that n+m=1,
or of the compounds of Formulae XIIIA, XIIIB and XIIIC 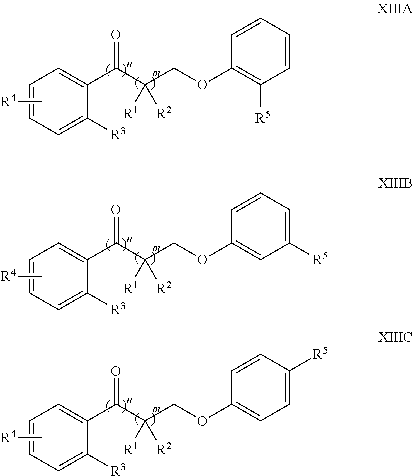
in which
R¹, R², R³, R⁴ and R⁵ have the meanings designated above,
n is equal to 0 or 1, m is equal to 0 or 1, provided that n+m=1.
or of the compounds of Formulae XIID, XIIE and XIIF 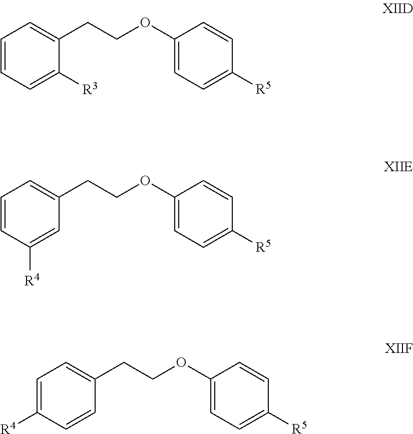
in which R³, R⁴ and R⁵ have the meanings designated above.

5. The method according to claim 1, of the compounds of formulae 1 to 137.

6. The method according to claim 1, of one or more compounds of Formula 1 for preparing cosmetic compositions in which the anti-ageing properties belong to the group constituted by reinforcement of the dermis, proliferation of dermal fibroblasts, production of collagen, inhibition of expression of the “matrix metallopeptidase” gene MMP9 and of the protein “heat shock protein beta-1” HSPB1,
or for preparing cosmetic compositions in which the depigmenting properties belong to the group constituted by anti-tyrosinase activity and anti-melanogenesis activity.

7. Cosmetic composition containing as active ingredient one or more compounds of formula 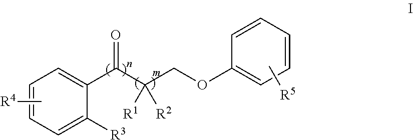
in which
n is equal to 0 or 1, m is equal to 0 or 1, provided that n+m=1,
R¹ and R², which may be identical or different, represent
hydrogen atoms,
halogen atoms selected from fluorine, chlorine, bromine or iodine,
linear or branched alkyl groups comprising from 1 to 10 carbon atoms, in particular the methyl, ethyl, isopropyl groups, optionally substituted with one or more halogens selected from fluorine, chlorine, bromine or iodine, with a —CF₃ group, with a hydroxy,
—OH groups,
linear or branched alkoxy groups comprising from 1 to 10 carbon atoms, in particular methoxy, ethoxy, optionally substituted with one or more halogens selected from fluorine, chlorine, bromine or iodine, and in particular the trifluoromethoxy group —OCF₃,
phenoxy groups —OPh,
aryloxy groups —OAr in which Ar represents an aromatic group comprising from 6 to 12 carbon atoms optionally substituted with one or more halogen atoms selected from fluorine, chlorine, bromine, or optionally substituted with one or more —OH groups in free or protected form, and in particular protected in the form of —OMes group, —OTHP group of formula 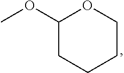
group derived from ethylene glycol of formula 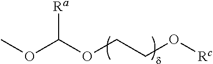
in which
δ varies from 1 to 12,
R^{a} represents a hydrogen or a linear or branched alkyl group comprising from 1 to 6 carbon atoms,
R^{c} represents a linear or branched alkyl group with 1 to 10 carbon atoms,
group derived from propylene glycol of formula, 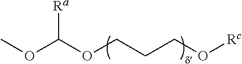
in which
δ′ varies from 1 to 5,
R^{a} and R^{c} have the meanings stated above,
group derived from a glycoside compound that may be α- or β-furanose or α- or β-pyranose,
siloxy group of formula —OSi(R^{a})₃ in which R^{a} has the meanings stated above,
—OSitBdPh group of formula 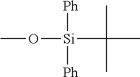 [SMILES: CO[Si](c1ccccc1)(c1ccccc1)C(C)(C)C]
—OSitBdM group of formula, 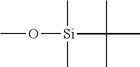
benzyloxy groups —OCH₂Ph,
alkenyl groups comprising from 2 to 10 carbon atoms, linear or branched and comprising at least one C═C double bond,
acyloxy groups comprising from 2 to 10 carbon atoms, derived from carboxylic acids,
—OTHP groups of the above formula,
—OMes groups,
groups derived from ethylene glycol of formula 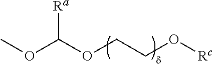
in which
δ varies from 1 to 12,
R^{a} represents a hydrogen or a linear or branched alkyl group comprising from 1 to 6 carbon atoms,
R^{c} represents a linear or branched alkyl group with 1 to 10 carbon atoms,
groups derived from propylene glycol of formula, 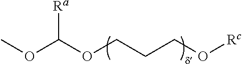
in which
δ′ varies from 1 to 5,
R^{a} and R^{c} have the meanings stated above,
—OH groups optionally coupled to glycoside compounds that may be α- or β-furanose or α- or β-pyranose,
siloxy groups of formula —OSi(R^{a})₃ in which R^{a} has the meanings stated above,
—OSitBdPh groups of formula 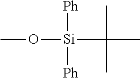 [SMILES: CO[Si](c1ccccc1)(c1ccccc1)C(C)(C)C]
—OSitBdM groups of formula, 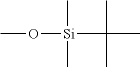
said R¹ and R² optionally forming a ring comprising 3 carbon atoms, said ring having the following formula 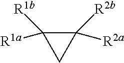
in which
R^{1a}, R^{1b}, R^{2a} and R^{2b}, which may be identical or different, represent
hydrogen atoms,
halogen atoms selected from fluorine, chlorine, bromine or iodine,
linear or branched alkyl groups comprising from 1 to 10 carbon atoms, in particular the methyl, ethyl, isopropyl groups, optionally substituted with one or more halogens selected from fluorine, chlorine, bromine or iodine, with a —CF₃ group, with a hydroxy,
provided that R³ is a hydroxy group —OH,
R³ and R⁴, which may be identical or different, represent
hydrogen atoms,
halogen atoms selected from fluorine, chlorine, bromine,
linear, branched or cyclic alkyl groups comprising from 1 to 10 carbon atoms, in particular the methyl, ethyl, isopropyl groups, optionally substituted with one or more halogens selected from fluorine, chlorine, bromine or iodine, with a —CF₃ group, with a hydroxy,
hydroxyl groups —OH,
linear or branched alkoxy groups comprising from 1 to 10 carbon atoms, in particular the methoxy, ethoxy, isopropoxy, tert-butoxy groups, optionally substituted with one or more halogens selected from fluorine, chlorine, bromine or iodine, and in particular the trifluoromethoxy group —OCF₃,
benzyloxy groups,
an alkenyloxy group comprising from 2 to 10 carbon atoms, linear or branched and comprising at least one C═C double bond,
an alkynyloxy group comprising from 2 to 10 carbon atoms, linear or branched and comprising at least one C≡C triple bond,
—OTHP groups of formula 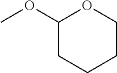
substituted —OTHP groups of the following formula 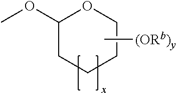
in which
x is equal to 0 or 1, y varies from 0 to 4,
R^{b} is a hydrogen or an acetyl —C(O)—CH₃,
groups derived from ethylene glycol of formula 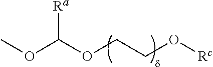
in which
δ varies from 1 to 12,
R^{a} represents a linear or branched alkyl group comprising from 1 to 6 carbon atoms,
R^{c} represents a linear or branched alkyl group with 1 to 10 carbon atoms,
groups derived from propylene glycol of formula 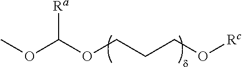
in which
δ′ varies from 1 to 5,
R^{a} and R^{c} have the meanings stated above,
—OH groups optionally coupled to a glycoside compound, which may be an α- or β-furanose or an α- or β-pyranose,
a siloxy group of formula —OSi(R^{a})₃ in which R^{a} has the meanings stated above,
—OSitBdPh of formula 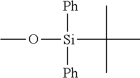 [SMILES: CO[Si](c1ccccc1)(c1ccccc1)C(C)(C)C]
—OSitBdM of formula, 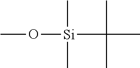
R⁵ represents
a hydrogen atom,
a halogen atom selected from fluorine, chlorine, bromine,
a linear, branched or cyclic alkyl group comprising from 1 to 10 carbon atoms, in particular a methyl, ethyl, isopropyl group, optionally substituted with one or more halogens selected from fluorine, chlorine, bromine or iodine, with a —CF₃ group, with a hydroxy,
an alkenyl group comprising from 2 to 6 carbon atoms, linear or branched and comprising at least one C═C double bond,
an alkynyl group comprising from 2 to 6 carbon atoms, linear or branched and comprising at least one C≡C triple bond,
a phenyl group optionally substituted with a halogen atom, a —CF₃ group, a linear or branched alkyl group comprising from 1 to 10 carbon atoms, a linear or branched alkoxy group comprising from 1 to 10 carbon atoms,
a hydroxyl group —OH,
a linear or branched alkoxy group comprising from 1 to 10 carbon atoms, in particular the methoxy, ethoxy, isopropoxy, tert-butoxy groups, optionally substituted with one or more halogens selected from fluorine, chlorine, bromine or iodine, and in particular the trifluoromethoxy group —OCF₃,
a benzyloxy group,
a phenoxy group,
a thiophenyl group,
an acyloxy group comprising from 2 to 10 carbon atoms, linear or branched,
an —OTHP group of formula 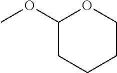
a substituted —OTHP group of the following formula 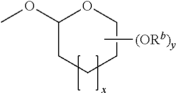
in which
x is equal to 0 or 1, y varies from 0 to 4,
R^{b} is a hydrogen or an acetyl —C(O)—CH₃,
an —OSO₂R^{c} group in which R^{c} is an alkyl group comprising from 1 to 6 carbon atoms, in particular methyl or ethyl, or an aromatic group comprising from 6 to 18 carbon atoms, optionally substituted with one or more halogen atoms, hydroxy or alkoxy group, nitro,
an —OSO₃M group in which M represents an Na⁺ or K⁺ ion,
a group derived from ethylene glycol of formula 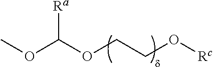
in which
δ varies from 1 to 12,
R^{a} represents a linear or branched alkyl group comprising from 1 to 6 carbon atoms,
R^{c} represents a linear or branched alkyl group with 1 to 10 carbon atoms,
a group derived from propylene glycol of formula 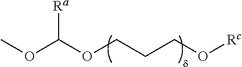
in which
δ′ varies from 1 to 5,
R^{a} and R^{c} have the meanings stated above,
an —OH group optionally coupled to a glycoside compound, which may be an α- or β-furanose or an α- or β-pyranose,
a siloxy group of formula —OSi(R^{a})₃ in which R^{a} has the meanings stated above,
—OSitBdPh of formula 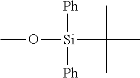 [SMILES: CO[Si](c1ccccc1)(c1ccccc1)C(C)(C)C]
—OSitBdM of formula, 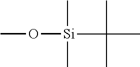
—COOH,
—CN,
—NH₂,
—NH₃⁺, X⁻
—NR^{d}R^{e},
—NHR^{d}R^{e+}, X⁻
—NHCOR^{f},
NHCOOR^{g},
—NO₂,
R^{d}, R^{e} representing linear or branched alkyl groups comprising from 1 to 4 carbon atoms, optionally substituted with one or more halogen atoms, or carbon chains interrupted by oxygen or sulphur atoms, benzyl groups optionally substituted with a halogen atom, a hydroxy group, an alkoxy group comprising from 1 to 8 carbon atoms,
R^{f} represents a linear or branched alkyl group containing from 1 to 4 carbon atoms, a phthalimido group (in this case NH is replaced with N), a benzyl group optionally substituted with a halogen atom, a hydroxy group, an alkoxy group and in particular substituted with the methoxy group in the para position,
R^{g} represents a linear or branched alkyl group comprising from 1 to 4 carbon atoms, optionally substituted with one or more halogen atoms, or a carbon chain interrupted by oxygen or sulphur atoms, a phenyl group, a benzyl group optionally substituted with a halogen atom, a hydroxy group, an alkoxy group and in particular substituted with the methoxy group in the para position,
X⁻ representing a halide ion,
a group derived from piperazine, in particular 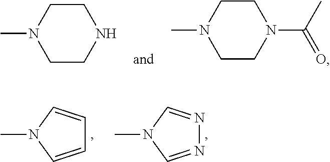
provided that R³ and R⁴ do not represent simultaneously —OH groups or groups comprising an oxygen atom bound to the phenyl, said active ingredient being combined with a cosmetically acceptable vehicle.

8. Cosmetic composition containing as active ingredient one or more compounds of Formula 1 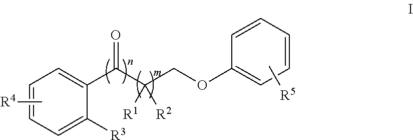
in which:
n is equal to 0 or 1, m is equal to 0 or 1, provided that n+m=1,
R¹ and R² have the meanings given in claim 1,
R³ and R⁴, which may be identical or different, represent:
hydrogen atoms,
hydroxyl groups,
and R⁵ represents:
a linear, branched or cyclic alkyl group comprising from 2 to 10 carbon atoms, in particular an ethyl, isopropyl group, optionally substituted with one or more halogens selected from fluorine, chlorine, bromine or iodine, with a —CF₃ group, with a hydroxy,
an alkenyl group comprising from 2 to 6 carbon atoms, linear or branched and comprising at least one C═C double bond,
an alkynyl group comprising from 2 to 6 carbon atoms, linear or branched and comprising at least one C≡C triple bond.
said active ingredient being combined with a cosmetically acceptable vehicle.

9. Cosmetic composition according to claim 7, constituted by a mixture comprising at least one compound of Formula 1 as active ingredient in combination with one or more compounds having anti-ageing and/or depigmenting properties, in particular in combination with fatty acids such as linoleic acid or azelaic acid, and/or with antioxidants such as vitamin C and/or with tocopherol derivatives, and/or with desquamation additives such as retinoic acid or glycolic acid,
said composition having anti-ageing and/or depigmenting properties.

10. Cosmetic composition according to claim 7, containing as active ingredient one or more compounds of formula 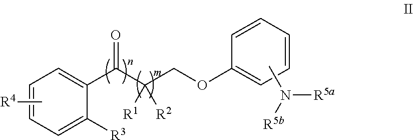
in which
n is equal to 0 or 1, m is equal to 0 or 1, provided that n+m=1,
R¹, R², R³ and R⁴ have the meanings designated above,
—NR^{5a}R^{5b} represents a group comprising a nitrogen atom fixed to the ring, optionally in the form of its salt, this group being selected from
—NH₂,
—NR^{d}R^{e},
—NHCOR^{f},
—NHCOOR^{g},
—NO₂,
a group derived from piperazine, in particular 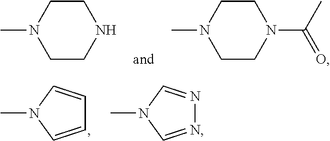
R^{d}, R^{e} representing linear or branched alkyl groups comprising from 1 to 4 carbon atoms, optionally substituted with one or more halogen atoms, or carbon chains interrupted by oxygen or sulphur atoms, benzyl groups optionally substituted with a halogen atom, a hydroxy group, an alkoxy group comprising from 1 to 8 carbon atoms,
R^{f} represents a linear or branched alkyl group containing from 1 to 4 carbon atoms, a phthalimido group (in this case NH is replaced with N), a benzyl group optionally substituted with a halogen atom, a hydroxy group, an alkoxy group and in particular substituted with the methoxy group in the para position,
R^{g} represents a linear or branched alkyl group comprising from 1 to 4 carbon atoms, optionally substituted with one or more halogen atoms, or a carbon chain interrupted by oxygen or sulphur atoms, a phenyl group, a benzyl group optionally substituted with a halogen atom, a hydroxy group, an alkoxy group and in particular substituted with the methoxy group in the para position,
in combination with a cosmetically acceptable vehicle,
or one or more compounds of Formula III 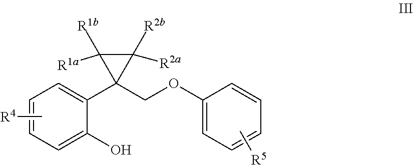
in which
R⁴ and R⁵ have the meanings designated above,
R^{1a}, R^{1b}, R^{2a} and R^{2b}, which may be identical or different, represent
hydrogen atoms,
halogen atoms selected from fluorine, chlorine, bromine or iodine,
linear or branched alkyl groups comprising from 1 to 10 carbon atoms, in particular the methyl, ethyl, isopropyl groups, optionally substituted with one or more halogens selected from fluorine, chlorine, bromine or iodine, with a —CF₃ group, with a hydroxy,
in combination with a cosmetically acceptable vehicle,
or one or more compounds of Formula 1V 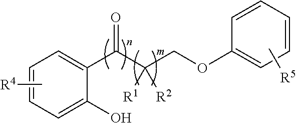
in which
R¹, R², R⁴ and R⁵ have the meanings designated above,
n is equal to 0 or 1, m is equal to 0 or 1, provided that n+m=1,
in combination with a cosmetically acceptable vehicle,
or one or more compounds of Formula 1VA 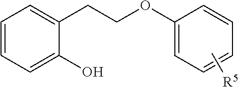
in which
R⁵ has the meaning designated above,
in combination with a cosmetically acceptable vehicle,
or one or more compounds of Formula V 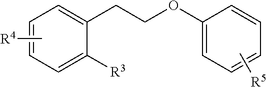
in which
R³, R⁴ and R⁵ have the meanings designated above, provided that R³ and R⁴ are both different from —OH or are of any form in which —OH is protected,
in combination with a cosmetically acceptable vehicle.

11. Cosmetic composition according to claim 7, containing as active ingredient, one or more of the compounds 1 to 137 in combination with a cosmetically acceptable vehicle.

12. Compound of Formula 1 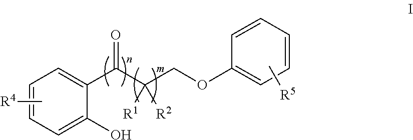
in which:
n is equal to 0 or 1, m is equal to 0 or 1, provided that n+m=1,
R¹ and R² have the meanings given in claim 1,
R³ and R⁴, which may be identical or different, represent:
hydrogen atoms,
hydroxyl groups,
and R⁵ represents:
a linear, branched or cyclic alkyl group comprising from 2 to 10 carbon atoms, in particular an ethyl, isopropyl group, optionally substituted with one or more halogens selected from fluorine, chlorine, bromine or iodine, with a —CF₃ group, with a hydroxy,
an alkenyl group comprising from 2 to 6 carbon atoms, linear or branched and comprising at least one C═C double bond,
an alkynyl group comprising from 2 to 6 carbon atoms, linear or branched and comprising at least one C≡C triple bond.

13. Compound of Formula X 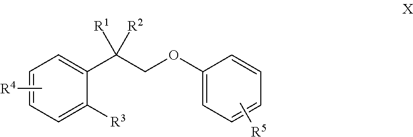
in which
n is equal to 0, and m is equal to 1,
R¹ and R² have the meanings given in claim 1,
R³ and R⁴, which may be identical or different, represent:
hydrogen atoms,
hydroxyl groups,
and R⁵ represents:
a linear, branched or cyclic alkyl group comprising from 2 to 10 carbon atoms, in particular an ethyl, isopropyl group, optionally substituted with one or more halogens selected from fluorine, chlorine, bromine or iodine, with a —CF₃ group, with a hydroxy,
an alkenyl group comprising from 2 to 6 carbon atoms, linear or branched and comprising at least one C═C double bond,
an alkynyl group comprising from 2 to 6 carbon atoms, linear or branched and comprising at least one C≡C triple bond.

14. Compounds of Formula II 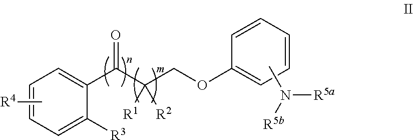
in which
n is equal to 0 or 1, m is equal to 0 or 1, provided that n+m=1,
R¹ and R² have the meanings given in claim 1,
R³ and R⁴ have the meanings given above, with the exception of the hydrogen atoms,
—NR^{5a}R^{5b} represents a group comprising a nitrogen atom fixed to the ring, optionally in the form of its salt, this group being selected from
—NH₂,
—NR^{d}R^{e},
—NHCOR^{f},
—NHCOOR^{g},
—NO₂,
a group derived from piperazine, in particular 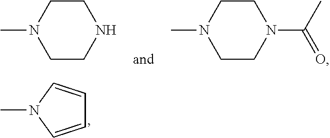 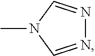
R^{d}, R^{e} representing linear or branched alkyl groups comprising from 1 to 4 carbon atoms, optionally substituted with one or more halogen atoms, or carbon chains interrupted by oxygen or sulphur atoms, benzyl groups optionally substituted with a halogen atom, a hydroxy group, an alkoxy group comprising from 1 to 8 carbon atoms,
R^{f} represents a linear or branched alkyl group containing from 1 to 4 carbon atoms, a phthalimido group (in this case NH is replaced with N), a benzyl group optionally substituted with a halogen atom, a hydroxy group, an alkoxy group and in particular substituted with the methoxy group in the para position,
R^{g} represents a linear or branched alkyl group comprising from 1 to 4 carbon atoms, optionally substituted with one or more halogen atoms, or a carbon chain interrupted by oxygen or sulphur atoms, a phenyl group, a benzyl group optionally substituted with a halogen atom, a hydroxy group, an alkoxy group and in particular substituted with the methoxy group in the para position.
These are compounds comprising a nitrogen-containing group, in particular nitro, amino, amido, borne by the —OAr group of the following general formula

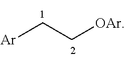

15. Compounds of Formula II 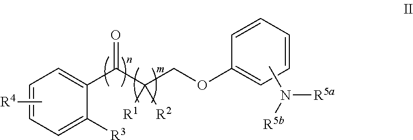
in which
n is equal to 0 or 1, m is equal to 0 or 1, provided that n+m=1,
R¹ and R² have the meanings given in claim 1,
R³ represents a hydroxyl group and R⁴ has the meanings given above,
—NR^{5a}R^{5b} represents a group comprising a nitrogen atom fixed to the ring, optionally in the form of its salt, this group being selected from
—NH₂,
—NR^{d}R^{e},
—NHCOR^{f},
—NHCOOR^{g},
—NO₂,
a group derived from piperazine, in particular 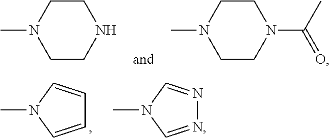
R^{d}, R^{e} representing linear or branched alkyl groups comprising from 1 to 4 carbon atoms, optionally substituted with one or more halogen atoms, or carbon chains interrupted by oxygen or sulphur atoms, benzyl groups optionally substituted with a halogen atom, a hydroxy group, an alkoxy group comprising from 1 to 8 carbon atoms,
R^{f} represents a linear or branched alkyl group containing from 1 to 4 carbon atoms, a phthalimido group (in this case NH is replaced with N), a benzyl group optionally substituted with a halogen atom, a hydroxy group, an alkoxy group and in particular substituted with the methoxy group in the para position,
R^{g} represents a linear or branched alkyl group comprising from 1 to 4 carbon atoms, optionally substituted with one or more halogen atoms, or a carbon chain
interrupted by oxygen or sulphur atoms, a phenyl group, a benzyl group optionally substituted with a halogen atom, a hydroxy group, an alkoxy group and in particular substituted with the methoxy group in the para position.
These are compounds comprising a nitrogen-containing group, in particular nitro, amino, amido, borne by the —OAr group of the following general formula

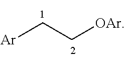

16. Compounds of Formula III 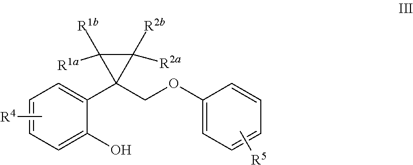
in which
R⁴ and R⁵ have the meanings designated in claim 1,
R^{1a}, R^{1b}, R^{2a} and R^{2b}, which may be identical or different, represent
hydrogen atoms,
halogen atoms selected from fluorine, chlorine, bromine or iodine,
linear or branched alkyl groups comprising from 1 to 10 carbon atoms, in particular the methyl, ethyl, isopropyl groups, optionally substituted with one or more halogens selected from fluorine, chlorine, bromine or iodine, with a —CF₃ group, with a hydroxy.

17. Compounds of formulae

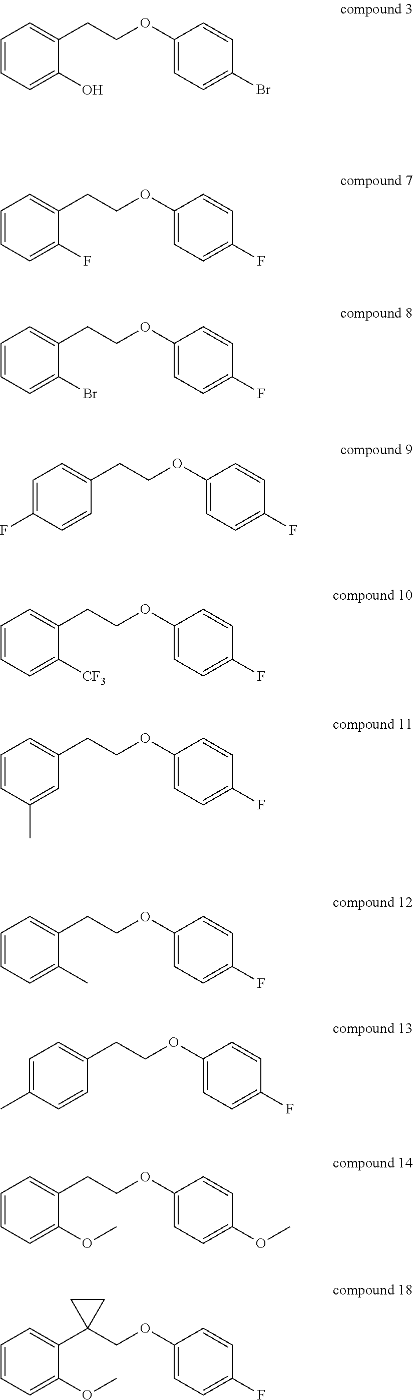

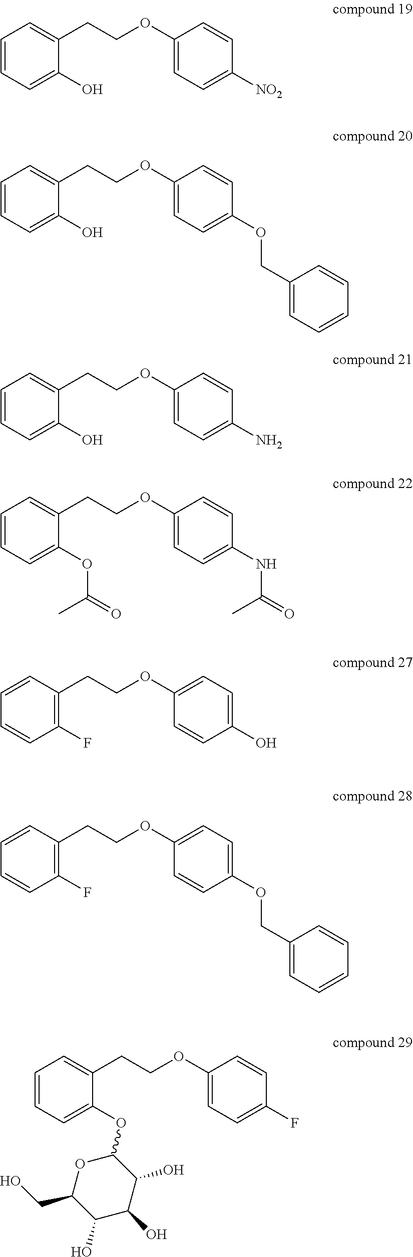

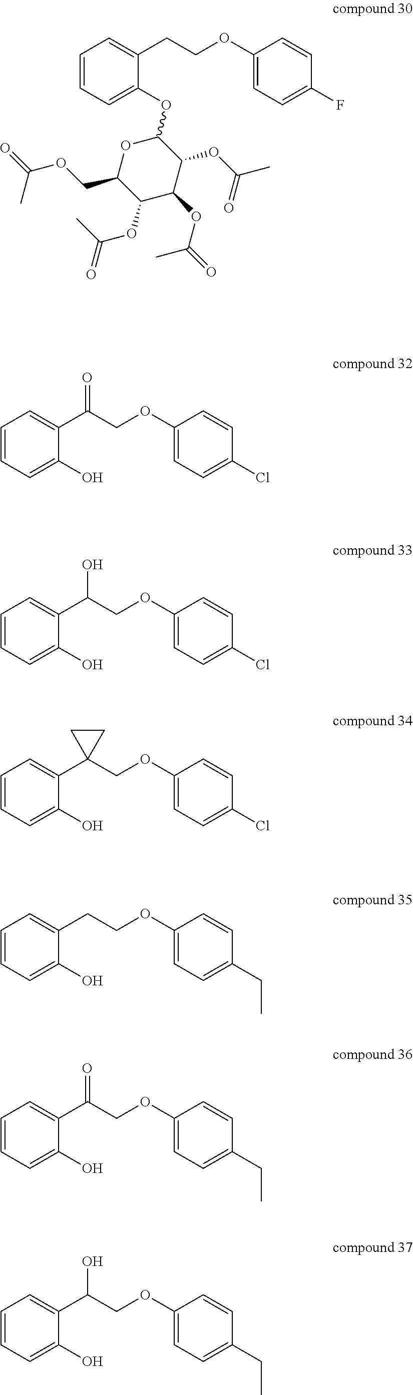

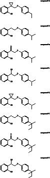

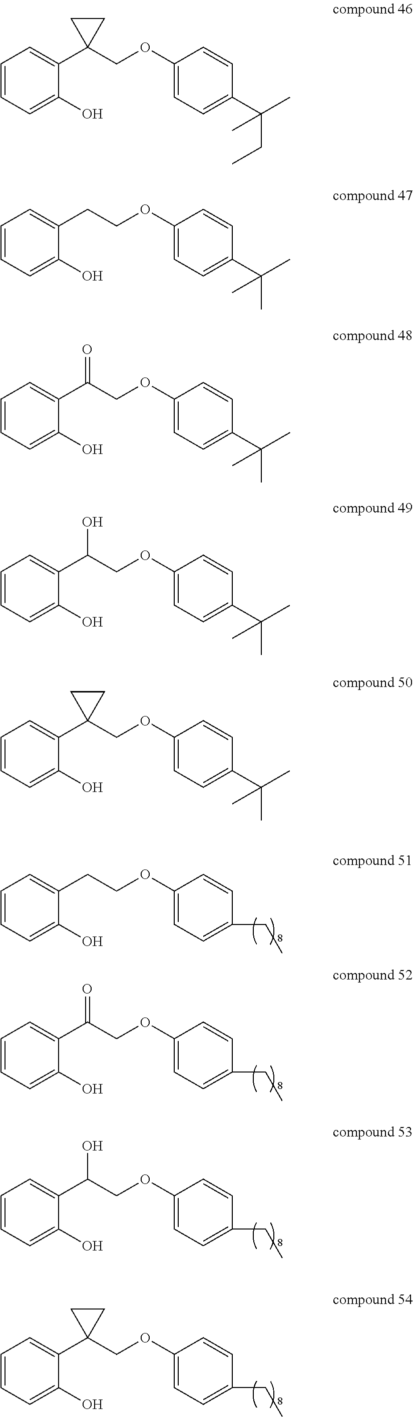

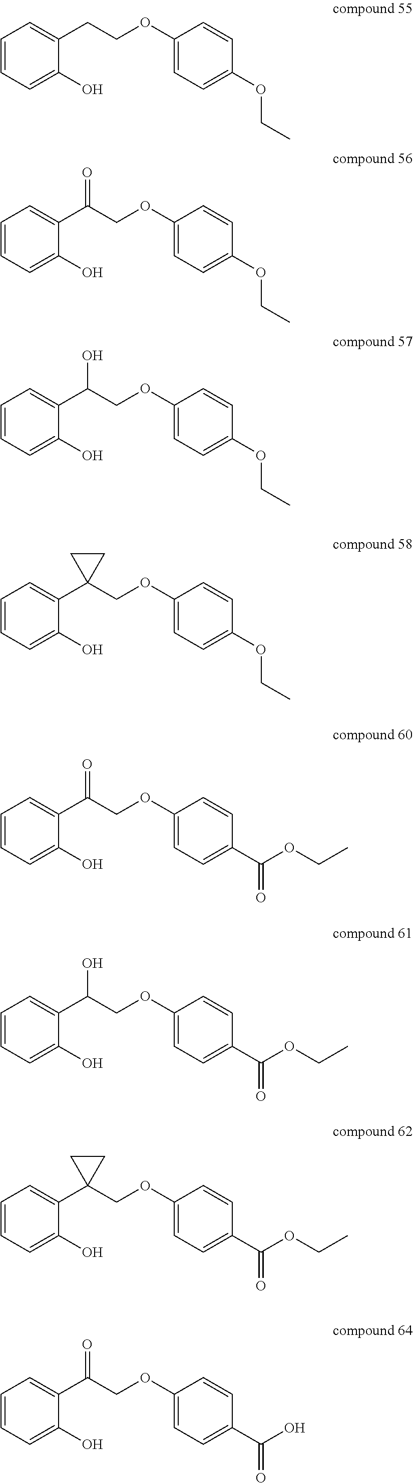

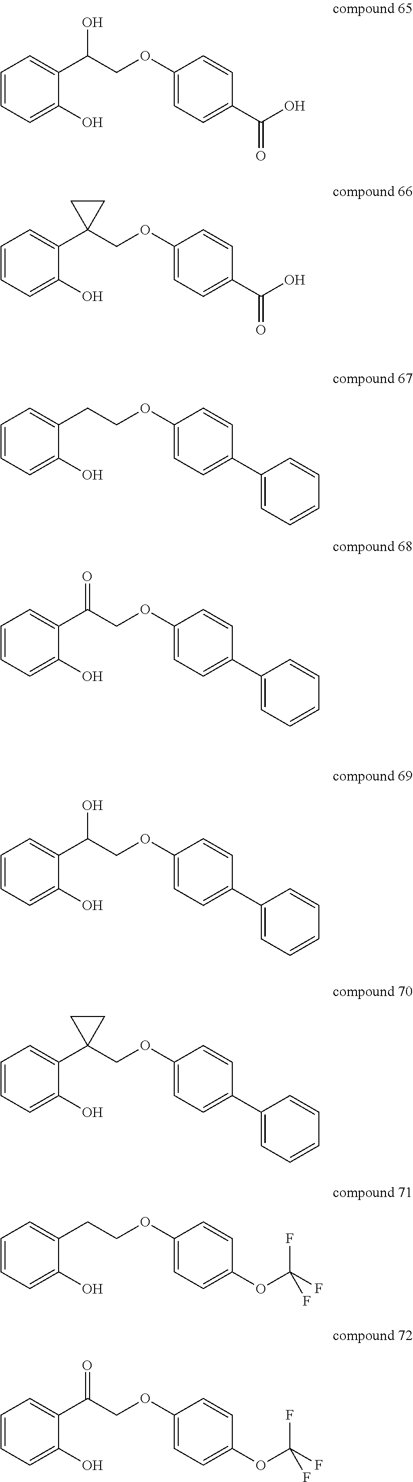

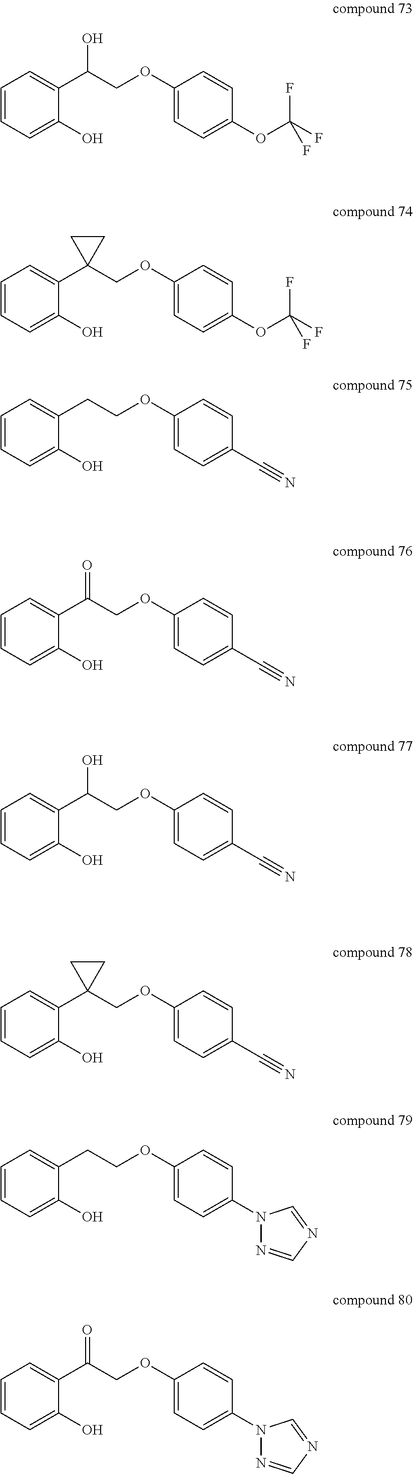

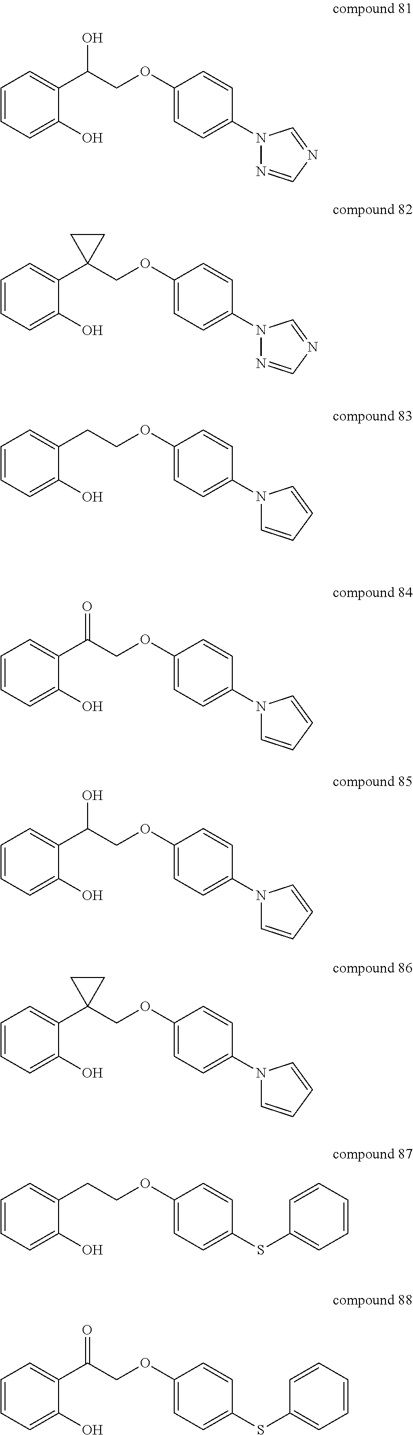

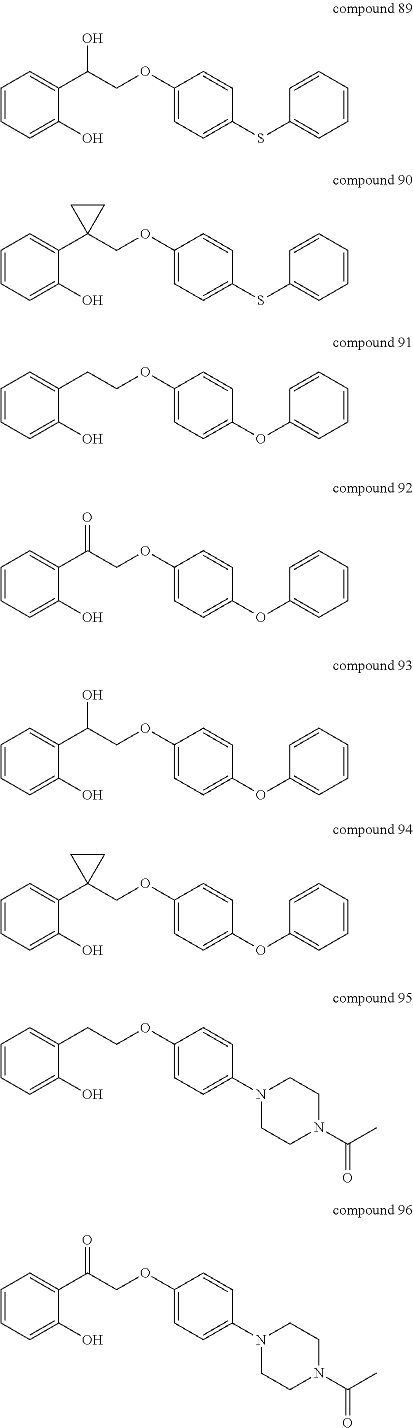

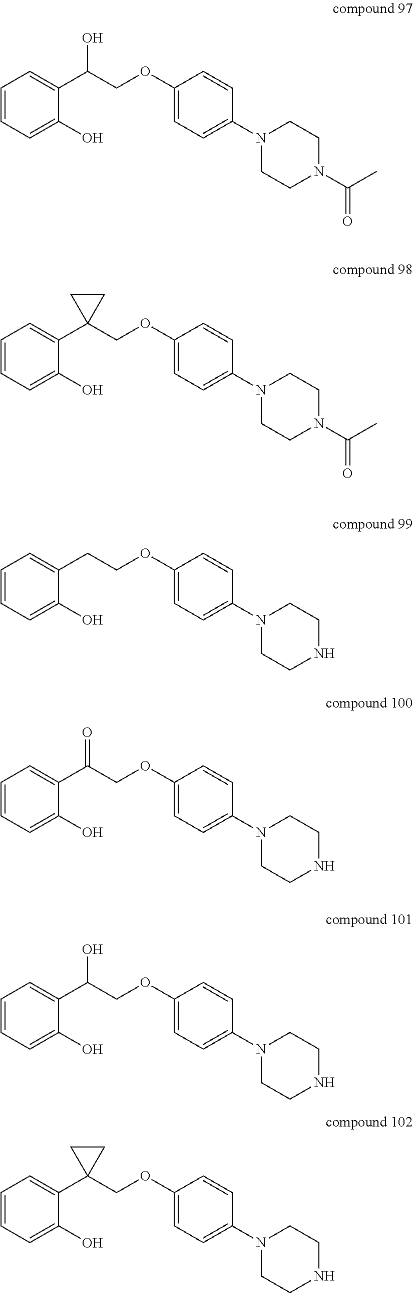 [SMILES: CC(=O)N1CCN(c2ccc(OCC(O)c3ccccc3O)cc2)CC1 ; CC(=O)N1CCN(c2ccc(OCC3(c4ccccc4O)CC3)cc2)CC1 ; Oc1ccccc1CCOc1ccc(N2CCNCC2)cc1 ; O=C(COc1ccc(N2CCNCC2)cc1)c1ccccc1O ; Oc1ccccc1C(O)COc1ccc(N2CCNCC2)cc1 ; Oc1ccccc1C1(COc2ccc(N3CCNCC3)cc2)CC1]

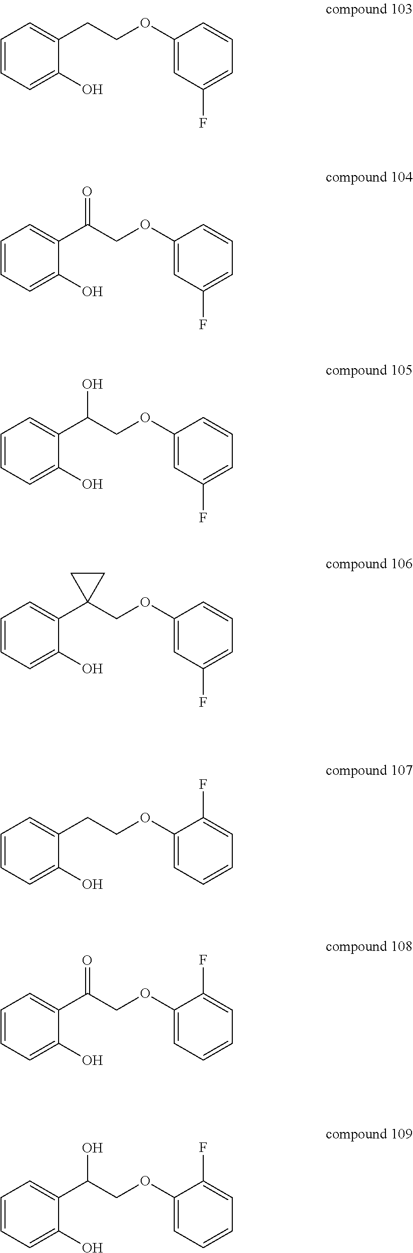

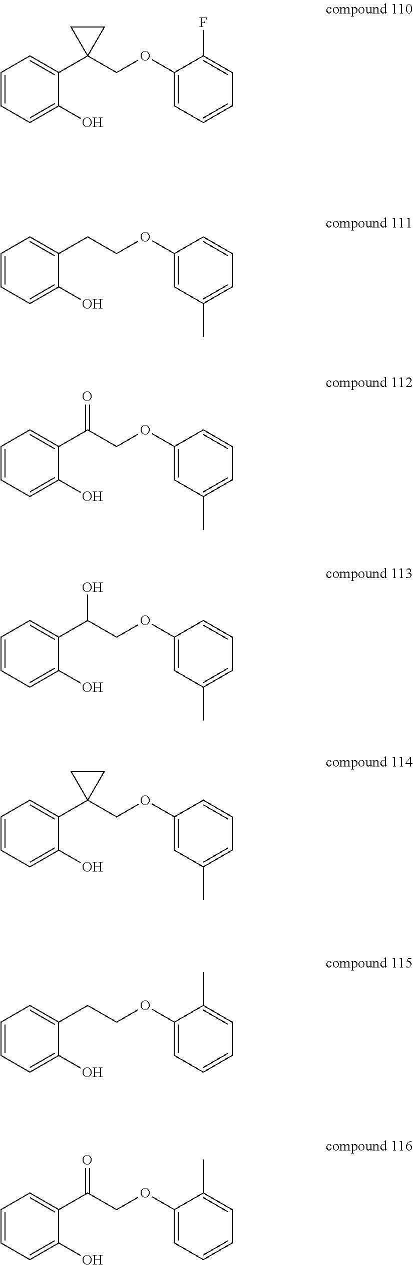

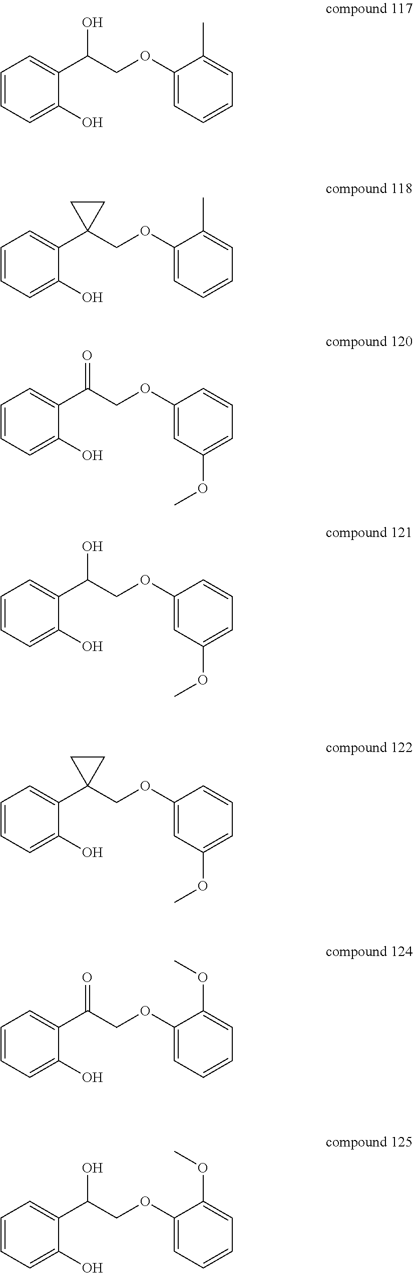

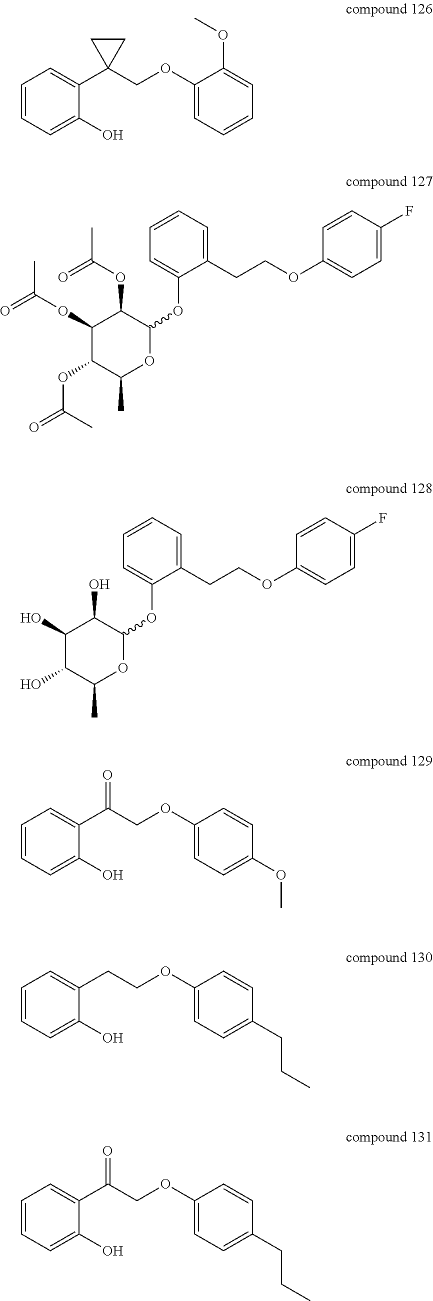 [SMILES: COc1ccccc1OCC1(c2ccccc2O)CC1 ; CC(=O)O[C@@H]1[C@@H](OC(C)=O)[C@@H](OC(C)=O)C(Oc2ccccc2CCOc2ccc(F)cc2)O[C@H]1C ; C[C@@H]1OC(Oc2ccccc2CCOc2ccc(F)cc2)[C@H](O)[C@H](O)[C@H]1O ; COc1ccc(OCC(=O)c2ccccc2O)cc1 ; CCCc1ccc(OCCc2ccccc2O)cc1 ; CCCc1ccc(OCC(=O)c2ccccc2O)cc1]

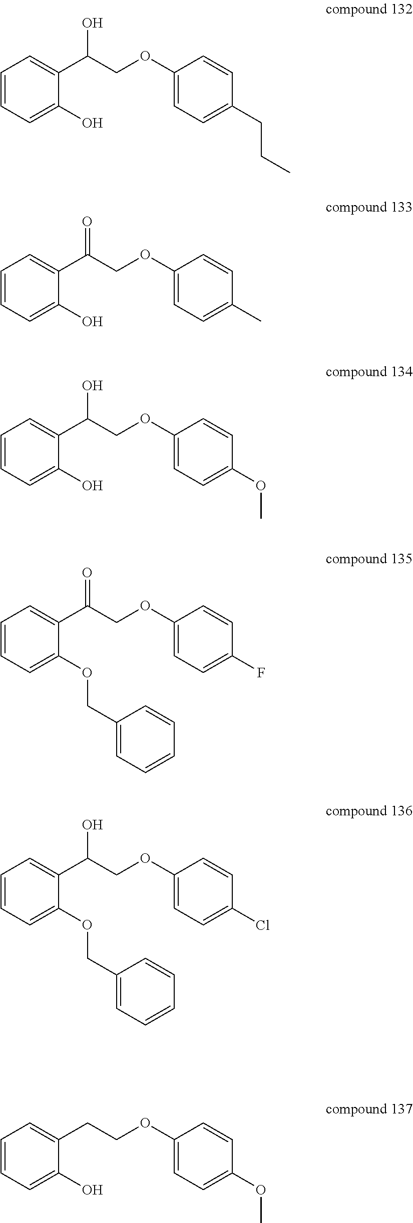 [SMILES: CCCc1ccc(OCC(O)c2ccccc2O)cc1 ; Cc1ccc(OCC(=O)c2ccccc2O)cc1 ; COc1ccc(OCC(O)c2ccccc2O)cc1 ; O=C(COc1ccc(F)cc1)c1ccccc1OCc1ccccc1 ; OC(COc1ccc(Cl)cc1)c1ccccc1OCc1ccccc1 ; COc1ccc(OCCc2ccccc2O)cc1]

18. Compound of formula [chemical structure]
